(19) United States
(12) Patent Application Publication
(10) Pub. No.: US 2025/0157040 A1
(43) Pub. Date: May 15, 2025

(54) SYSTEMS AND METHODS FOR DETERMINING EYE CLOSURE STATUS

(71) Applicant: BLINKTBI, INC., Charleston, SC (US)

(72) Inventors: Logan Sean TEDER, Charleston, SC (US); Andrew MASSEY, Charleston, SC (US); Alexis JACKSON, Charleston, SC (US)

(21) Appl. No.: 18/837,527

(22) PCT Filed: Feb. 10, 2023
(86) PCT No.: PCT/US2023/062439

§ 371 Date: Aug. 9, 2024

Related U.S. Application Data
(60) Provisional application No. 63/309,256, filed on Feb. 11, 2022.

(51) Int. Cl. G06T 7/00; G06V 10/75; G06V 10/766; G06V 10/82; G06V 40/18

(52) U.S. Cl. CPC G06T 7/0014; G06V 10/751; G06V 10/766; G06V 10/82; G06V 40/18; G06T 2207/10016; G06T 2207/20081; G06T 2207/20084; G06T 2207/30041; G06V 2201/03

(57) ABSTRACT

Systems and methods for determining eye closure status of a subject are provided. A first lower eyelid trace and a first upper eyelid trace are obtained, where each respective eyelid trace comprises, for each time increment in a plurality of consecutive time increments, a respective location of the respective eyelid of a first eye of the subject. A first minimum difference and a first maximum difference between the location of the upper eyelid and the lower eyelid are obtained, across the time increments that are between a first and second time increment in the plurality of consecutive time increments. The first minimum difference and a difference between the first maximum difference and the first minimum difference is passed through an activation function, obtaining a first result. The first result is used to provide an eye closure status of the first eye.

### CROSS REFERENCE TO RELATED APPLICATIONS

This application claims priority to U.S. Provisional Patent Application No. 63/309,256, entitled “Systems and Methods for Determining Eye Closure Status,” filed Feb. 11, 2022, the content of which is hereby incorporated by reference, in its entirety, for all purposes.

### TECHNICAL FIELD

This specification describes determining eye closure status using an eyelid trace.

### BACKGROUND

Blinking is an activity that serves physiologic maintenance, protective, and brain restorative functions. With every blink, an ocular lubricant is swept across the eyeball providing moisture and anti-microbial protection to the orbit. Blinking also provides rest and reset for retinal cells that are activated and fade when fixed on an object. Blinks can be classified as voluntary, acquired, passive, spontaneous and reflexive; each with a distinct cause, innervation pathway and temporal profile. Reflexive blinks, also called the blink reflexes, can be elicited by tactile, light, and sound stimulation, and serve to provide a first line of defense to the globe and the brain behind it.

Numerous studies have examined the blink reflex in normal populations, as well as the changes that occur in a variety of neurological conditions, such as Parkinson's disease, Huntington's disease, schizophrenia and severe traumatic brain injury. Indeed, the sensitivity of the blink reflex to a variety of insults indicates that it may be a useful tool for assessing neurological function. This is particularly relevant given the current public interest in developing methods to detect concussions, offering a more encompassing field sobriety test, and monitoring patients for early onset of Alzheimer's disease.

The blink reflex is a primitive brainstem response to an external stimulus, such as air, visual cues or electrical signals, which can be affected by multiple neurological disorders, including those that affect the dopaminergic circuit that controls the eyelid. Previous studies using electromyography have shown that diffuse axonal injury and exercise result in measurable changes in the blink reflex. As described above, previous studies have also examined the changes that occur to the blink reflex in a variety of neurological conditions, such as Parkinson's disease, Huntington's disease, schizophrenia and severe traumatic brain injury. See, for example, Garner et al., 2018, “Blink reflex parameters in baseline, active, and head-impact Division I athletes,” Cogent Engineering, 5:1429110; doi: 10.1080/23311916.2018.1429110.

However, despite the correlation between altered blink reflex parameters and neurological health, quantitative approaches for examining the blink reflex have not been adopted in clinical practice, and qualitative assessments remain standard. In recent decades, considerable technological advancements have been made. For instance, digital image capture technologies have improved significantly over the past two decades and have significantly decreased in cost. The high frame rates currently available allow for measurements in the millisecond range, where image capture occurs at speeds that facilitate the quantification of blink reflexes.

Concurrently, processing power has increased, making analysis of the thousands of image frames recorded at high frame rates feasible. High-speed image capture has been successfully employed to record and measure eyelid location during a single, voluntary blink in both healthy subjects and in patients with blepharoptosis. However, no study has used high-speed image capture to study reflexive blinks. Furthermore, methods that use non-invasive measurements and machine learning-based image analysis to measure eyelid location, or to diagnose a variety of neurological conditions, are lacking. See, for example, Tsai et al., 2017, “Development of a Non-Invasive Blink Reflexometer,” IEEE J Transl Eng Health Med; 5:3800204; doi: 10.1109/JTEHM.2017.2782669, which is hereby incorporated by reference.

Given the above background, there is a need in the art for systems and methods of processing and analyzing images to obtain eyelid traces, and of using eyelid traces to determine eye closure status during a blink.

### SUMMARY

Advantageously, the present disclosure provides robust techniques for determining eye closure status. The following presents a summary of the invention in order to provide a basic understanding of some of the aspects of the invention. This summary is not an extensive overview of the invention. It is not intended to identify key/critical elements of the invention or to delineate the scope of the invention. Its sole purpose is to present some of the concepts of the invention in a simplified form as a prelude to the more detailed description that is presented later.

Accordingly, one aspect of the present disclosure provides a method for determining an eye closure status of a respective subject. The method comprises obtaining, in electronic format, a first lower eyelid trace, where the first lower eyelid trace comprises, for each respective time increment in a plurality of consecutive time increments, a respective location of a lower eyelid of a first eye of the respective subject. The method also includes obtaining, in electronic format, a first upper eyelid trace, where the first upper eyelid trace comprises, for each respective time increment in the plurality of consecutive time increments, a respective location of an upper eyelid of the first eye.

The method includes obtaining, between a first and second time increment within the plurality of consecutive time increments, a first minimum difference between the location of the upper eyelid and the lower eyelid of the first eye across the time increments in the plurality of consecutive time increments that are between the first and second time increment. The method also includes obtaining, between the first and second time increment within the plurality of consecutive time increments, a first maximum difference between the location of the upper eyelid and the lower eyelid of the first eye across the time increments in the plurality of consecutive time increments that are between the first and second time increment. The first minimum difference and a difference between the first maximum difference and the first minimum difference is passed through an activation function (e.g., a sigmoid function) thereby obtaining a first result, and the first result is used to provide an eye closure status of the first eye.

In some embodiments, the eye closure status of the first eye is a first Boolean status indicator of whether or not the first eye experienced an eye blink at any point between the first and second time increment. The first eye is deemed to have experienced an eye blink at a point between the first and second time increment when the first result satisfies a first threshold, and the first eye is deemed to have not experienced an eye blink at any point between the first and second time increment when the first result fails to satisfy the first threshold. In some embodiments, the threshold is between 0.80 and 0.97. In some embodiments, the threshold is between 0.89 and 0.95.

In some embodiments, the first and second time increment are between 50 milliseconds and 500 milliseconds apart from each other.

In some embodiments, the method further comprises obtaining, in electronic format, a second lower eyelid trace, where the second lower eyelid trace comprises, for each respective time increment in the plurality of consecutive time increments, a respective location of a lower eyelid of a second eye of the respective subject; and obtaining, in electronic format, a second upper eyelid trace, where the second upper eyelid trace comprises, for each respective time increment in the plurality of consecutive time increments, a respective location of an upper eyelid of the second eye. The method further includes obtaining, between a first and second time increment within the plurality of consecutive time increments, a second minimum difference between the location of the upper eyelid and the lower eyelid of the second eye across the time increments in the plurality of consecutive time increments that are between the first and second time increment; and obtaining, between the first and second time increment within the plurality of consecutive time increments, a second maximum difference between the location of the upper eyelid and the lower eyelid of the second eye across the time increments in the plurality of consecutive time increments that are between the first and second time increment. The method further comprises passing a square of the second minimum difference divided by a difference between the second maximum difference and the second minimum difference through an activation function (e.g., a sigmoid function) thereby obtaining a second result; and using the second result to provide an eye closure status of the second eye.

In some embodiments, an involuntary eye stimulus occurs at a time point between the first and second time increment, and the first time increment is a first predetermined amount of time prior to the involuntary eye stimulus and the second time increment is a second predetermined amount of time after the involuntary eye stimulus.

In some embodiments, the first predetermined amount of time is between 5 milliseconds and 30 milliseconds, and the second predetermined amount of time is between 75 milliseconds and 150 milliseconds.

In some embodiments, the involuntary eye stimulus is directed to the first eye or the second eye and the method further comprises reporting out whether the involuntary eye stimulus is directed to the first eye or the second eye along with an indication as to whether the involuntary eye stimulus was directed to the first eye or the second eye.

In some embodiments, the method further comprises, prior to the obtaining a lower eyelid trace, generating the involuntary eye stimulus.

In some embodiments, the involuntary eye stimulus is a puff of air directed to the first eye or the second eye. In some embodiments, the involuntary eye stimulus is a flash of light directed to the first eye or the second eye.

In some embodiments, the method further comprises repeating the obtaining (a), the obtaining (b), the obtaining (c), the obtaining (d), the passing (e), and the using (f), for each respective subject in a plurality of subjects.

In some embodiments, the plurality of subjects is 50 or more subjects, 100 or more subjects, 1000 or more subjects, 10,000 or more subjects, or 100,000 or more subjects.

In some embodiments, the activation function normalizes the first result to a value between 0 and 1. In some embodiments, the activation function is a logistic sigmoid function.

In some embodiments, the plurality of consecutive time increments consists of between 20 time increments and 1000 time increments. In some embodiments, each time increment in the plurality of consecutive time increments represents between 1 millisecond and 10 milliseconds of time.

In some embodiments, the method further comprises generating the first lower eyelid trace by a procedure comprising, for each respective time increment in the plurality of consecutive time increments: (i) obtaining a corresponding image of the first eye comprising a corresponding plurality of pixels and one or more pixel values for each pixel in the corresponding plurality of pixels, and (ii) inputting the corresponding image into a trained neural network comprising 10,000 or more parameters, thereby obtaining the respective location of the upper eyelid of the first eye at the respective time increment.

In some embodiments, the neural network comprises a plurality of convolutional layers, where each convolutional layer in the plurality of convolutional layers comprises one or more filters, a respective size, and a respective stride, and one or more pooling layers, where each pooling layer in the one or more pooling layers comprises a respective size and a respective stride. In some embodiments, the neural network is LeNet, AlexNet, VGGNet 16, GoogLeNet, ResNet, SE-ResNeXt, MobileNet, or EfficientNet.

In some embodiments, an edge length, in pixels, of the corresponding image consists of between 164 pixels and 1024 pixels.

In some embodiments, the neural network comprises an initial convolutional neural network layer that receives a grey-scaled pixel value for each pixel in the corresponding plurality of pixels as input into the neural network, where the initial convolutional neural network layer includes a first activation function, and where the initial convolutional neural network layer convolves the corresponding plurality of pixels into more than 10 separate parameters for each pixel in the corresponding plurality of pixels.

In some embodiments, the neural network further comprises a pooling layer that pools the 10 separate parameters for each pixel in the plurality of pixels outputted by the initial convolutional neural network layer.

In some embodiments, the initial convolutional neural network layer has a stride of two or more.

In some embodiments, the neural network further comprises a plurality of intermediate blocks including a first intermediate block and a final intermediate block, where the first intermediate block takes as input the output of the pooling layer, each intermediate block in the plurality of intermediate blocks other than the first intermediate block and the final intermediate block takes, as input, an output of another intermediate block in the plurality of intermediate blocks and has an output that serves as input to another intermediate block in the plurality of intermediate blocks, and each intermediate block comprises a respective first convolutional layer comprising more than 1000 parameters, where the respective convolutional layer has a corresponding activation function.

In some embodiments, each intermediate block in the plurality of intermediate blocks comprises a corresponding second convolutional layer that takes, as input, an output of the respective first convolutional layer.

In some embodiments, each intermediate block in the plurality of intermediate blocks comprises a merge layer that merges (i) an output of the respective second convolutional layer and (ii) an output of a preceding intermediate block in the plurality of intermediate blocks.

In some embodiments, each intermediate block in the plurality of intermediate blocks has a corresponding input size and a corresponding output size, and, when the corresponding input size of a respective intermediate block differs from the corresponding output size, the respective intermediate block further comprises a corresponding third convolutional layer that receives, as input, the (ii) output of the preceding intermediate block, where the corresponding third convolutional layer convolves the (ii) output of the preceding intermediate block prior to the merging (i) and (ii) by the merge layer.

In some embodiments, the final intermediate block takes, as input, an output of another intermediate block in the plurality of intermediate blocks and produces, as output, a flattened data structure comprising a predetermined plurality of values.

In some embodiments, the neural network further comprises a regressor block including a first dropout layer, a first linear layer, and a corresponding activation function, wherein the regressor block takes, as input, the flattened data structure comprising the predetermined plurality of values.

In some embodiments, the first dropout layer removes a first subset of values from the plurality of values in the flattened data structure, based on a first dropout rate. In some embodiments, the first linear layer applies a first linear transformation to the plurality of values in the flattened data structure.

In some embodiments, the regressor block further includes a second dropout layer, where the second dropout layer removes a second subset of values from the plurality of values in the flattened data structure, based on a second dropout rate. In some embodiments, the regressor block further includes a second linear layer, wherein the second linear layer applies a second linear transformation to the plurality of values in the flattened data structure.

In some embodiments, the first activation function is tanh, sigmoid, softmax, Gaussian, Boltzmann-weighted averaging, absolute value, linear, rectified linear unit (ReLU), bounded rectified linear, soft rectified linear, parameterized rectified linear, average, max, min, sign, square, square root, multiquadric, inverse quadratic, inverse multiquadric, polyharmonic spline, swish, mish, Gaussian error linear unit (GeLU), scaled exponential linear unit (SELU), or thin plate spline.

In some embodiments, the regressor block produces, as output, a corresponding first calculated set of coordinates that localize the upper eyelid in the corresponding image.

In some embodiments, the method further comprises using the eye closure status of the first eye to diagnose a condition of the respective subject. In some such embodiments, the condition is a neurological condition such as, but not limited to, Parkinson's disease, Huntington's disease, schizophrenia, or a traumatic brain injury. In some embodiments, the condition is Alzheimer's disease.

In some embodiments the condition is a level of sobriety. That is, in some such embodiments, the eye closure status determined using the methods of the present disclosure can be used to determine whether a respective subject is sober or not. In other embodiments, the eye closure status determined using the methods of the present disclosure can be used to determine a degree of intoxication, such as alcohol intoxication.

Another aspect of the present disclosure provides a computing system including one or more processors and memory storing one or more programs that further comprise instructions for performing any of the above-disclosed methods alone or in combination.

Another aspect of the present disclosure provides non-transitory computer-readable storage medium comprising one or more programs in which the one or more programs further comprise instructions for performing any of the above-disclosed methods alone or in combination. The one or more programs are configured for execution by a computer.

Various embodiments of systems, methods, and devices within the scope of the appended claims each have several aspects, no single one of which is solely responsible for the desirable attributes described herein. Without limiting the scope of the appended claims, some prominent features are described herein. After considering this discussion, and particularly after reading the section entitled “Detailed Description” one will understand these and other features and attributes of various embodiments of the present disclosure and their advantageous applications and/or uses.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### BRIEF DESCRIPTION OF THE DRAWINGS

The implementations disclosed herein are illustrated by way of example, and not by way of limitation, in the figures of the accompanying drawings. Like reference numerals refer to corresponding parts throughout the several views of the drawings.

FIG. 1 illustrates an example block diagram illustrating a computing device in accordance with some embodiments of the present disclosure.

FIGS. 2A, 2B, 2C, and 2D collectively illustrate an example flowchart of a method for determining an eye closure status of a respective subject, in which dashed boxes represent optional steps, in accordance with some embodiments of the present disclosure.

FIGS. 3A and 3B collectively illustrate an example flowchart of a method for generating a lower eyelid trace, in which dashed boxes represent optional steps, in accordance with some embodiments of the present disclosure.

FIGS. 4A and 4B illustrate example performance measures of a pre-trained ResNet model (FIG. 4A) and a ResNet model with custom architecture (FIG. 4B), in accordance with some embodiments of the present disclosure.

FIGS. 5A, 5B, and 5C illustrate example images used as input to a neural network, in accordance with some embodiments of the present disclosure.

FIG. 6 collectively illustrates example images used as input to an untrained or partially trained neural network, where each image comprises a corresponding first measured set of coordinates that localize an upper eyelid and a corresponding second measured set of coordinates that localize a lower eyelid, in accordance with some embodiments of the present disclosure.

FIG. 7 illustrates an example neural network for handling data encoded into channels, in accordance with some embodiments of the present disclosure.

FIGS. 8A, 8B, 8C, and 8D illustrate example outputs of an auxiliary neural network for determining a class of an image, in accordance with some embodiments of the present disclosure.

FIG. 9 is an example diagram that plots the difference between the location of an upper eyelid and the location of a lower eyelid of an eye across a plurality of time increments, in accordance with some embodiments of the present disclosure.

### DETAILED DESCRIPTION

The implementations described herein provide various technical solutions for determining eye closure status using eyelid traces. As described above, blinking is indicative of physiologic maintenance, protective, and brain restorative functions. The blink reflex can also be used to assess neurological function, such as in instances where a patient is experiencing or is likely to experience a neurological condition or impairment (e.g., Parkinson's disease, Huntington's disease, schizophrenia and/or severe traumatic brain injury).

Advancements in image processing technology, including high-speed image capture and machine learning-based image analysis, offer new avenues for quantitative assessment of blinks and the blink reflex. However, human eye blinks exhibit particular physical qualities that can hinder the accurate detection of blinks when using machine learning-based or algorithm-based technologies. For instance, studies have shown that most normal blinks are incomplete, such that the upper and lower eyelids do not physically touch across the full length of the eyelid. Instead, the upper eyelid is positioned to slightly overlap the lower eyelid at a distance that allows the upper and lower tear films to coalesce, thus reestablishing the tear layer thickness over the entire ocular surface. See, for example, McMonnies, “Diagnosis and remediation of blink inefficiency,” Contact Lens and Anterior Eye 44(3):101331 (2021), doi: 10.1016/j.clae.2020.04.015, which is hereby incorporated herein by reference in its entirety.

Visual assessment of eye blinks by human observers generally involves a qualitative determination of whether a respective eye has completed a blink or not, e.g., whether a top lid has touched, or appears to touch, a bottom lid. This holistic approach, however, is difficult to recapitulate when using machine learning-based approaches that often rely upon quantitative determinations, where partial closures can serve to confound the model's ability to accurately distinguish between a partially open eye (e.g., no blink) and a normal blink where the eyelids do not fully close.

Nevertheless, reliance on human ability for blink assessment is impractical, if not impossible, in cases where the source data is obtained from high-speed image capture and includes thousands, tens of thousands, hundreds of thousands, or millions of frames. Moreover, human assessment proves further insufficient in cases involving a large number of instances or subjects, such as when preparing training datasets for machine learning using thousands, tens of thousands, hundreds of thousands, or millions of training objects.

Given the above background, there is a need in the art for improved systems and methods for determining eye closure status that overcome the abovementioned limitations and encompass instances in which the upper and lower eyelids do not fully close while blinking.

Accordingly, the present disclosure provides systems and methods for determining an eye closure status of an eye of a respective subject. A first lower eyelid trace and a first upper eyelid trace are obtained, where each respective eyelid trace includes, for each respective time increment in a plurality of consecutive time increments, a respective location of the respective eyelid. A minimum difference between the location of the upper and lower eyelids is obtained across the time increments between a first and second time increment within the plurality of consecutive time increments (e.g., within a time window). A maximum difference between the upper and lower eyelids is also obtained for the time window. A square of the minimum difference divided by a difference between the maximum difference and the minimum difference is passed through an activation function thereby obtaining a first result, and the first result is used to provide an eye closure status of the eye.

In an exemplary embodiment, the upper and lower eyelid traces are considered within a small time window (e.g., approximately 110 milliseconds) immediately prior to and succeeding an involuntary eye stimulus (e.g., a puff of air and/or a flash of light). Referring to the difference between these two traces at each time point within the time window, the minimum difference and the maximum difference within the time window are identified, and the minimum value is squared and divided by the sample range between the maximum difference and the minimum difference. This non-negative number is passed into an activation function (e.g., a sigmoid function), after which closure can be ascertained as a Boolean by thresholding the output. In some embodiments, when the output is less than the threshold value, the eye is deemed to have blinked, and when the output is greater than or equal to the threshold value, the eye is deemed not to have blinked. In some embodiments, the output is used to determine a degree to which the eye closed during the time window (e.g., a value between 0 and 1 indicating the degree to which the eye closed, where a value of 0 indicates full closure and a value of 1 indicates no closure response).

Advantageously, the presently disclosed systems and methods overcome the abovementioned limitations by providing an automated method for determination of eye closure status that can be used for batch processing of numerous frames, scans, and/or subjects. As such, the time and effort required by laborious, cumbersome, and in some cases infeasible manual techniques are removed. The systems and methods are repeatable and deterministic and can be applied to a plurality of instances in a systematic manner, avoiding issues of subjectivity that can arise with human visual assessment. Furthermore, the inputs and outputs are machine-readable and compatible with models for classification and detection, which can be used to develop automated pipelines for evaluation of blink reflexes and associated neurological conditions.

Advantageously, the present disclosure further provides various systems and methods for determining eye closure status using the location of an upper and/or a lower eyelid in an image of a subject, where eyelid location is determined computationally using a model for more accurate image processing and analysis. The complexity of a machine learning model includes time complexity (running time, or the measure of the speed of an algorithm for a given input size n), space complexity (space requirements, or the amount of computing power or memory needed to execute an algorithm for a given input size n), or both. Complexity (and subsequent computational burden) applies to both training of and prediction by a given model.

In some instances, computational complexity is impacted by implementation, incorporation of additional algorithms or cross-validation methods, and/or one or more parameters (e.g., weights and/or hyperparameters). In some instances, computational complexity is expressed as a function of input size n, where input data is the number of instances (e.g., the number of training samples), dimensions p (e.g., the number of features), the number of trees nₜᵣₑₑₛ (e.g., for methods based on trees), the number of support vectors nₛᵥ (e.g., for methods based on support vectors), the number of neighbors k (e.g., for k nearest neighbor algorithms), the number of classes c, and/or the number of neurons nᵢ at a layer i (e.g., for neural networks). With respect to input size n, then, an approximation of computational complexity (e.g., in Big O notation) denotes how running time and/or space requirements increase as input size increases. Functions can increase in complexity at slower or faster rates relative to an increase in input size. Various approximations of computational complexity include but are not limited to constant (e.g., O(1)), logarithmic (e.g., O(log n)), linear (e.g., O(n)), loglinear (e.g., O(n log n)), quadratic (e.g., O(n²)), polynomial (e.g., O(n^{c})), exponential (e.g., O(cⁿ)), and/or factorial (e.g., O(n!)). In some instances, simpler functions are accompanied by lower levels of computational complexity as input sizes increase, as in the case of constant functions, whereas more complex functions such as factorial functions can exhibit substantial increases in complexity in response to slight increases in input size.

Computational complexity of machine learning models can similarly be represented by functions (e.g., in Big O notation), and complexity may vary depending on the type of model, the size of one or more inputs or dimensions, usage (e.g., training and/or prediction), and/or whether time or space complexity is being assessed. For example, complexity in decision tree algorithms is approximated as O(n²p) for training and O(p) for predictions, while complexity in linear regression algorithms is approximated as O(p²n+p³) for training and O(p) for predictions. For random forest algorithms, training complexity is approximated as O(n²pnₜᵣₑₑₛ) and prediction complexity is approximated as O(pnₜᵣₑₑₛ). For gradient boosting algorithms, complexity is approximated as O(npnₜᵣₑₑₛ) for training and O(pnₜᵣₑₑₛ) for predictions. For kernel support vector machines, complexity is approximated as O(n²p+n³) for training and O(nₛᵥp) for predictions. For naïve Bayes algorithms, complexity is represented as O(np) for training and O(p) for predictions, and for neural networks, complexity is approximated as O(pn₁+n₁n₂+ . . . ) for predictions. Complexity in K nearest neighbors algorithms is approximated as O(knp) for time and O(np) for space. For logistic regression algorithms, complexity is approximated as O(np) for time and O(p) for space. For logistic regression algorithms, complexity is approximated as O(np) for time and O(p) for space.

As described above, for machine learning models, computational complexity dictates the scalability and therefore the overall effectiveness and usability of a model (e.g., a classifier) for increasing input, feature, and/or class sizes, as well as for variations in model architecture. In the context of large-scale image processing, as in the case of analysis of the thousands of image frames recorded at high frame rates using high-speed image capture described above, the computational complexity of functions performed on large image datasets (e.g., batches of images for a plurality of training subjects) may strain the capabilities of many existing systems. In addition, as the number of input features (e.g., number of pixels (e.g., image size and/or resolution), augmentations, and/or channels) and/or the number of instances (e.g., training subjects, test subjects, and/or number of images per subject (e.g., frame rate and/or batch size)) increases together with technological advancements and expanding downstream applications and possibilities, the computational complexity of any given classification model can quickly overwhelm the time and space capacities provided by the specifications of a respective system.

Thus, by using a machine learning model with a minimum input size (e.g., at least 10,000, at least 20,000, or at least 100,000 pixels) and/or a minimum number of parameters (e.g., at least 1000, at least 10,000, or at least 100,000 parameters) to obtain a location of an eyelid in an image of a subject, the computational complexity is proportionally increased such that it cannot be mentally performed, and the method addresses a computational problem.

Additional details on computational complexity in machine learning models are provided in “Computational complexity of machine learning algorithms,” published Apr. 16, 2018, available online at: thekemeltrip.com/machine/learning/computational-complexity-learning-algorithms; Hastie, 2001, The Elements of Statistical Learning, Springer, New York; and Arora and Barak, 2009, Computational Complexity: A Modern Approach, Cambridge University Press, New York; each of which is hereby incorporated herein by reference in its entirety.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, components, circuits, and networks have not been described in detail so as not to unnecessarily obscure aspects of the embodiments.

### Definitions

As used herein, the term “about” or “approximately” means within an acceptable error range for the particular value as determined by one of ordinary skill in the art, which depends in part on how the value is measured or determined, e.g., the limitations of the measurement system. For example, in some embodiments “about” means within 1 or more than 1 standard deviation, per the practice in the art. In some embodiments, “about” means a range of 20%, ±10%, ±5%, or ±1% of a given value. In some embodiments, the term “about” or “approximately” means within an order of magnitude, within 5-fold, or within 2-fold, of a value. Where particular values are described in the application and claims, unless otherwise stated the term “about” meaning within an acceptable error range for the particular value can be assumed. All numerical values within the detailed description herein are modified by “about” the indicated value, and consider experimental error and variations that would be expected by a person having ordinary skill in the art. The term “about” can have the meaning as commonly understood by one of ordinary skill in the art. In some embodiments, the term “about” refers to ±10%. In some embodiments, the term “about” refers to ±5%.

As disclosed herein, the term “subject,” “training subject,” or “test subject” refers to any living or non-living organism, including but not limited to a human (e.g., a male human, female human, fetus, pregnant female, child, or the like) and/or a non-human animal. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale, and shark. The terms “subject” and “patient” are used interchangeably herein and can refer to a human or non-human animal who is known to have, or potentially has, a medical condition or disorder, such as, e.g., a neurological condition. In some embodiments, a subject is a “normal” or “control” subject, e.g., a subject that is not known to have a medical condition or disorder. In some embodiments, a subject is a male or female of any stage (e.g., a man, a woman, or a child).

A subject from whom an image is obtained, or who is stimulated, measured, and/or diagnosed using any of the methods or systems described herein can be of any age and can be an adult, infant or child. In some cases, the subject is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 years old, or within a range therein (e.g., between about 2 and about 20 years old, between about 20 and about 40 years old, or between about 40 and about 90 years old).

Subjects (e.g., patients) that can benefit from a method of the present disclosure include subjects that are suspected of having a neurological condition (e.g., Alzheimer's disease, Parkinson's disease, dementia, Huntington's disease, schizophrenia, migraines, stroke, and/or epilepsy). Subjects that can benefit from a method of the present disclosure further include subjects who have experienced or are suspected of having experienced a traumatic event, a head impact, or a mild traumatic brain injury (e.g., a concussion). Additionally, subjects that can benefit from a method of the present disclosure further include subjects who are participants in sports and recreational activities (e.g., athletes).

As used herein, the terms “control,” “reference,” and “normal” describe a subject and/or an image from a subject that does not have a particular condition (e.g., a neurological condition), has a baseline condition (e.g., prior to onset of the particular condition), or is otherwise healthy. In an example, a method as disclosed herein can be performed to diagnose a subject having a neurological condition using a trained neural network, where the neural network is trained using one or more training images obtained from the subject prior to the onset of the condition (e.g., at an earlier time point), or from a different, healthy subject. A control image can be obtained from a control subject, or from a database.

The term “normalize” as used herein means transforming a value or a set of values to a common frame of reference for comparison purposes. For example, when one or more pixel values corresponding to one or more pixels in a respective image are “normalized” to a predetermined statistic (e.g., a mean and/or standard deviation of one or more pixel values across one or more images), the pixel values of the respective pixels are compared to the respective statistic so that the amount by which the pixel values differ from the statistic can be determined.

As used herein, the term “untrained model” (e.g., “untrained classifier” and/or “untrained neural network”) refers to a machine learning model or algorithm, such as a classifier or a neural network, that has not been trained on a target dataset. In some embodiments, “training a model” (e.g., “training a neural network”) refers to the process of training an untrained or partially trained model (e.g., “an untrained or partially trained neural network”). For instance, consider the case of a plurality of training objects comprising a corresponding plurality of images discussed below. The plurality of images are applied as collective input to an untrained or partially trained neural network, in conjunction with a corresponding measured set of coordinates for each respective image (hereinafter “training dataset”) to train the untrained or partially trained neural network on coordinates that provide a respective location of an eyelid, thereby obtaining a trained classifier. Moreover, it will be appreciated that the term “untrained model” does not exclude the possibility that transfer learning techniques are used in such training of the untrained or partially trained model. For instance, Fernandes et al., 2017, “Transfer Learning with Partial Observability Applied to Cervical Cancer Screening,” Pattern Recognition and Image Analysis: 8^{th} Iberian Conference Proceedings, 243-250, which is hereby incorporated by reference, provides non-limiting examples of such transfer learning. In instances where transfer learning is used, the untrained classifier described above is provided with additional data over and beyond that of the primary training dataset. That is, in non-limiting examples of transfer learning embodiments, the untrained classifier receives (i) the plurality of images and the measured sets of coordinates for each respective image (“primary training dataset”) and (ii) additional data. Typically, this additional data is in the form of parameters (e.g., coefficients, weights, and/or hyperparameters) that were learned from another, auxiliary training dataset. Moreover, while a description of a single auxiliary training dataset has been disclosed, it will be appreciated that there is no limit on the number of auxiliary training datasets that may be used to complement the primary training dataset in training the untrained model in the present disclosure. For instance, in some embodiments, two or more auxiliary training datasets, three or more auxiliary training datasets, four or more auxiliary training datasets or five or more auxiliary training datasets are used to complement the primary training dataset through transfer learning, where each such auxiliary dataset is different than the primary training dataset. Any manner of transfer learning may be used in such embodiments. For instance, consider the case where there is a first auxiliary training dataset and a second auxiliary training dataset in addition to the primary training dataset. The parameters learned from the first auxiliary training dataset (by application of a first classifier to the first auxiliary training dataset) may be applied to the second auxiliary training dataset using transfer learning techniques (e.g., a second classifier that is the same or different from the first classifier), which in turn may result in a trained intermediate classifier whose parameters are then applied to the primary training dataset and this, in conjunction with the primary training dataset itself, is applied to the untrained classifier. Alternatively, a first set of parameters learned from the first auxiliary training dataset (by application of a first classifier to the first auxiliary training dataset) and a second set of parameters learned from the second auxiliary training dataset (by application of a second classifier that is the same or different from the first classifier to the second auxiliary training dataset) may each individually be applied to a separate instance of the primary training dataset (e.g., by separate independent matrix multiplications) and both such applications of the parameters to separate instances of the primary training dataset in conjunction with the primary training dataset itself (or some reduced form of the primary training dataset such as principal components or regression coefficients learned from the primary training set) may then be applied to the untrained classifier in order to train the untrained classifier. In some instances, additionally or alternatively, knowledge regarding eyelid location (e.g., an upper eyelid and/or a lower eyelid, etc.) derived from an auxiliary training dataset is used, in conjunction with the coordinate-labeled images in the primary training dataset, to train the untrained model.

As used interchangeably herein, the term “classifier” or “model” refers to a machine learning model or algorithm.

In some embodiments, a classifier is an unsupervised learning algorithm. One example of an unsupervised learning algorithm is cluster analysis.

In some embodiments, a classifier is supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, GradientBoosting, mixture models, hidden Markov models, Gaussian NB algorithms, linear discriminant analysis, or any combinations thereof. In some embodiments, a classifier is a multinomial classifier algorithm. In some embodiments, a classifier is a 2-stage stochastic gradient descent (SGD) model. In some embodiments, a classifier is a deep neural network (e.g., a deep-and-wide sample-level classifier).

Neural networks. In some embodiments, the classifier is a neural network (e.g., a convolutional neural network and/or a residual neural network). Neural network algorithms, also known as artificial neural networks (ANNs), include convolutional and/or residual neural network algorithms (deep learning algorithms). Neural networks can be machine learning algorithms that may be trained to map an input data set to an output data set, where the neural network comprises an interconnected group of nodes organized into multiple layers of nodes. For example, the neural network architecture may comprise at least an input layer, one or more hidden layers, and an output layer. The neural network may comprise any total number of layers, and any number of hidden layers, where the hidden layers function as trainable feature extractors that allow mapping of a set of input data to an output value or set of output values. As used herein, a deep learning algorithm (DNN) can be a neural network comprising a plurality of hidden layers, e.g., two or more hidden layers. Each layer of the neural network can comprise a number of nodes (or “neurons”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, xᵢ, and their associated parameters. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in analyzing an image of an eye of a subject. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used for analyzing an image of a subject in accordance with the present disclosure.

For instance, a deep neural network classifier comprises an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network classifier. In some embodiments, at least 100 parameters, at least 1000 parameters, at least 2000 parameters or at least 5000 parameters are associated with the deep neural network classifier. As such, deep neural network classifiers require a computer to be used because they cannot be mentally solved. In other words, given an input to the classifier, the classifier output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference.

Neural network algorithms, including convolutional neural network algorithms, suitable for use as classifiers are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as classifiers are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety. Additional example neural networks suitable for use as classifiers are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety.

Support vector machines. In some embodiments, the classifier is a support vector machine (SVM). SVM algorithms suitable for use as classifiers are described in, for example, Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5th Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data with a hyper-plane that is maximally distant from the labeled data. For cases in which no linear separation is possible, SVMs can work in combination with the technique of ‘kernels’, which automatically realizes a non-linear mapping to a feature space. The hyper-plane found by the SVM in feature space can correspond to a non-linear decision boundary in the input space. In some embodiments, the plurality of parameters (e.g., weights) associated with the SVM define the hyper-plane. In some embodiments, the hyper-plane is defined by at least 10, at least 20, at least 50, or at least 100 parameters and the SVM classifier requires a computer to calculate because it cannot be mentally solved.

Naïve Bayes algorithms. In some embodiments, the classifier is a Naive Bayes algorithm. Naive Bayes classifiers suitable for use as classifiers are disclosed, for example, in Ng et al., 2002, “On discriminative vs. generative classifiers: A comparison of logistic regression and naive Bayes,” Advances in Neural Information Processing Systems, 14, which is hereby incorporated by reference. A Naive Bayes classifier is any classifier in a family of “probabilistic classifiers” based on applying Bayes' theorem with strong (naive) independence assumptions between the features. In some embodiments, they are coupled with Kernel density estimation. See, for example, Hastie et al., 2001, The elements of statistical learning: data mining, inference, and prediction, eds. Tibshirani and Friedman, Springer, New York, which is hereby incorporated by reference.

Nearest neighbor algorithms. In some embodiments, a classifier is a nearest neighbor algorithm. Nearest neighbor classifiers can be memory-based and include no classifier to be fit. For nearest neighbors, given a query point x₀ (a test subject), the k training points x₍ᵣ₎, r, . . . , k (here the training subjects) closest in distance to x₀ are identified and then the point x₀ is classified using the k nearest neighbors. Here, the distance to these neighbors is a function of the abundance values of the discriminating gene set. In some embodiments, Euclidean distance in feature space is used to determine distance as d₍ᵢ₎=∥x₍ᵢ₎−x_{(O)}∥. Typically, when the nearest neighbor algorithm is used, the abundance data used to compute the linear discriminant is standardized to have mean zero and variance 1. The nearest neighbor rule can be refined to address issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, New York, each of which is hereby incorporated by reference.

A k-nearest neighbor classifier is a non-parametric machine learning method in which the input consists of the k closest training examples in feature space. The output is a class membership. An object is classified by a plurality vote of its neighbors, with the object being assigned to the class most common among its k nearest neighbors (k is a positive integer, typically small). If k=1, then the object is simply assigned to the class of that single nearest neighbor. See, Duda et al., 2001, Pattern Classification, Second Edition, John Wiley & Sons, which is hereby incorporated by reference. In some embodiments, the number of distance calculations needed to solve the k-nearest neighbor classifier is such that a computer is used to solve the classifier for a given input because it cannot be mentally performed.

Random forest, decision tree, and boosted tree algorithms. In some embodiments, the classifier is a decision tree. Decision trees suitable for use as classifiers are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety. In some embodiments, the decision tree classifier includes at least 10, at least 20, at least 50, or at least 100 parameters (e.g., weights and/or decisions) and requires a computer to calculate because it cannot be mentally solved.

Regression. In some embodiments, the classifier uses a regression algorithm. A regression algorithm can be any type of regression. For example, in some embodiments, the regression algorithm is logistic regression. In some embodiments, the regression algorithm is logistic regression with lasso, L2 or elastic net regularization. In some embodiments, those extracted features that have a corresponding regression coefficient that fails to satisfy a threshold value are pruned (removed from) consideration. In some embodiments, a generalization of the logistic regression model that handles multicategory responses is used as the classifier. Logistic regression algorithms are disclosed in Agresti, An Introduction to Categorical Data Analysis, 1996, Chapter 5, pp. 103-144, John Wiley & Son, New York, which is hereby incorporated by reference. In some embodiments, the classifier makes use of a regression model disclosed in Hastie et al., 2001, The Elements of Statistical Learning, Springer-Verlag, New York. In some embodiments, the logistic regression classifier includes at least 10, at least 20, at least 50, at least 100, or at least 1000 parameters (e.g., weights) and requires a computer to calculate because it cannot be mentally solved.

Linear discriminant analysis algorithms. Linear discriminant analysis (LDA), normal discriminant analysis (NDA), or discriminant function analysis can be a generalization of Fisher's linear discriminant, a method used in statistics, pattern recognition, and machine learning to find a linear combination of features that characterizes or separates two or more classes of objects or events. The resulting combination can be used as the classifier (linear classifier) in some embodiments of the present disclosure.

Mixture model and Hidden Markov model. In some embodiments, the classifier is a mixture model, such as that described in McLachlan et al., Bioinformatics 18(3):413-422, 2002. In some embodiments, in particular, those embodiments including a temporal component, the classifier is a hidden Markov model such as described by Schliep et al., 2003, Bioinformatics 19(1):i255-i263.

Clustering. In some embodiments, the classifier is an unsupervised clustering model. In some embodiments, the classifier is a supervised clustering model. Clustering algorithms suitable for use as classifiers are described, for example, at pages 211-256 of Duda and Hart, Pattern Classification and Scene Analysis, 1973, John Wiley & Sons, Inc., New York, (hereinafter “Duda 1973”) which is hereby incorporated by reference in its entirety. The clustering problem can be described as one of finding natural groupings in a dataset. To identify natural groupings, two issues can be addressed. First, a way to measure similarity (or dissimilarity) between two samples can be determined. This metric (e.g., similarity measure) can be used to ensure that the samples in one cluster are more like one another than they are to samples in other clusters. Second, a mechanism for partitioning the data into clusters using the similarity measure can be determined. One way to begin a clustering investigation can be to define a distance function and to compute the matrix of distances between all pairs of samples in the training set. If distance is a good measure of similarity, then the distance between reference entities in the same cluster can be significantly less than the distance between the reference entities in different clusters. However, clustering may not use a distance metric. For example, a nonmetric similarity function s(x, x′) can be used to compare two vectors x and x′. s(x, x′) can be a symmetric function whose value is large when x and x′ are somehow “similar.” Once a method for measuring “similarity” or “dissimilarity” between points in a dataset has been selected, clustering can use a criterion function that measures the clustering quality of any partition of the data. Partitions of the data set that extremize the criterion function can be used to cluster the data. Particular exemplary clustering techniques that can be used in the present disclosure can include, but are not limited to, hierarchical clustering (agglomerative clustering using a nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering. In some embodiments, the clustering comprises unsupervised clustering (e.g., with no preconceived number of clusters and/or no predetermination of cluster assignments).

Ensembles of classifiers and boosting. In some embodiments, an ensemble (two or more) of classifiers is used. In some embodiments, a boosting technique such as AdaBoost is used in conjunction with many other types of learning algorithms to improve the performance of the classifier. In this approach, the output of any of the classifiers disclosed herein, or their equivalents, is combined into a weighted sum that represents the final output of the boosted classifier. In some embodiments, the plurality of outputs from the classifiers is combined using any measure of central tendency known in the art, including but not limited to a mean, median, mode, a weighted mean, weighted median, weighted mode, etc. In some embodiments, the plurality of outputs is combined using a voting method. In some embodiments, a respective classifier in the ensemble of classifiers is weighted or unweighted.

The term “classification” can refer to any number(s) or other characters(s) that are associated with a particular property of a sample. For example, a “+” symbol (or the word “positive”) can signify that a sample is classified as having a desired outcome or characteristic. In another example, the term “classification” refers to a respective outcome or characteristic (e.g., closed, open, partial open). In some embodiments, the classification is binary (e.g., positive or negative) or has more levels of classification (e.g., a scale from 1 to 10 or 0 to 1). In some embodiments, the terms “cutoff” and “threshold” refer to predetermined numbers used in an operation. In one example, a cutoff value refers to a value above which results are excluded. In some embodiments, a threshold value is a value above or below which a particular classification applies. Either of these terms can be used in either of these contexts.

As used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. As such, the algorithms, models, regressors, and/or classifiers of the present disclosure cannot be mentally performed. In some embodiments n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

Several aspects are described below with reference to example applications for illustration. Numerous specific details, relationships, and methods are set forth to provide a full understanding of the features described herein. The features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are used to implement a methodology in accordance with the features described herein.

### Exemplary System Embodiments.

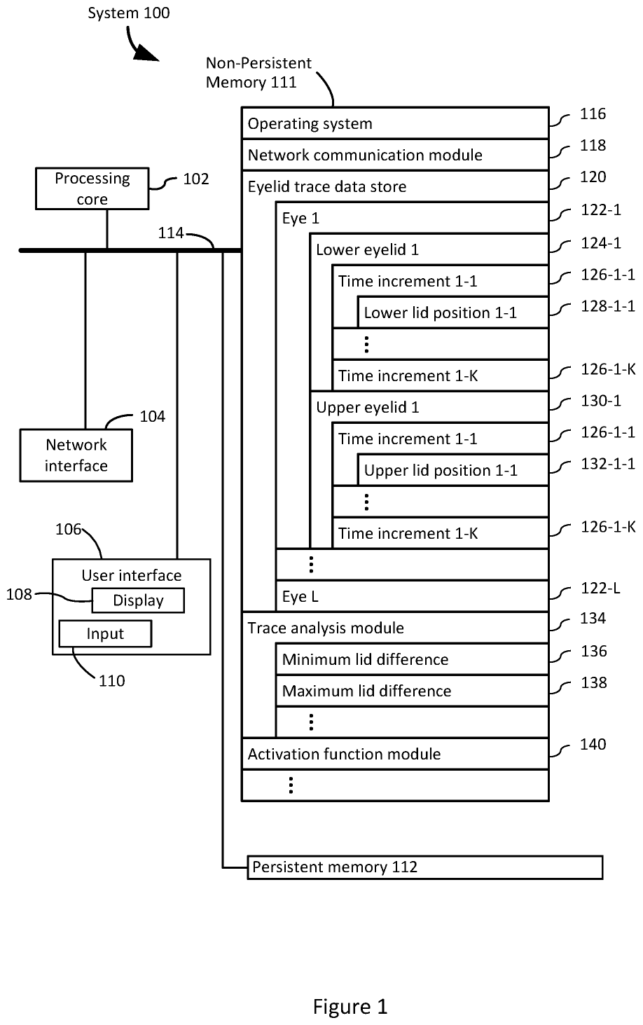

Details of an exemplary system are now described in conjunction with FIG. 1. FIG. 1 is a block diagram illustrating system 100 in accordance with some implementations. System 100 in some implementations includes one or more processing units CPU(s) 102 (also referred to as processors or processing core), one or more network interfaces 104, user interface 106, display 108, input 110, non-persistent memory 111, persistent memory 112, and one or more communication buses 114 for interconnecting these components. One or more communication buses 114 optionally include circuitry (sometimes called a chipset) that interconnects and controls communications between system components. Non-persistent memory 111 typically includes high-speed random access memory, such as DRAM, SRAM, DDR RAM, ROM, EEPROM, flash memory, whereas persistent memory 112 typically includes CD-ROM, digital versatile disks (DVD) or other optical storage, magnetic cassettes, magnetic tape, magnetic disk storage or other magnetic storage devices, magnetic disk storage devices, optical disk storage devices, flash memory devices, or other non-volatile solid-state storage devices. Persistent memory 112 optionally includes one or more storage devices remotely located from the CPU(s) 102. Persistent memory 112, and the non-volatile memory device(s) within non-persistent memory 112, comprise non-transitory computer-readable storage medium. In some implementations, non-persistent memory 111 or alternatively non-transitory computer-readable storage medium stores the following programs, modules and data structures, or a subset thereof, sometimes in conjunction with persistent memory 112:
instructions, programs, data, or information associated with an optional operating system 116, which includes procedures for handling various basic system services and for performing hardware dependent tasks;
instructions, programs, data, or information associated with an optional network communication module 118 for connecting the system 100 with other devices, or a communication network;
instructions, programs, data, or information associated with an eyelid trace data store 120 that includes, for at least a first eye 122 (e.g., 122-1, . . . 122-L) of a respective subject,
   a first lower eyelid trace for a lower eyelid 124 (e.g., 124-1) of the first eye 122-1 comprising, for each respective time increment 126 (e.g., 126-1-1, . . . 126-1-K) in a plurality of consecutive time increments, a respective location 128 (e.g., 128-1-1) of the lower eyelid, and
   a first upper eyelid trace for an upper eyelid 130 (e.g., 130-1) of the first eye 122-1 comprising, for each respective time increment 126 (e.g., 126-1-1, . . . 126-1-K) in a plurality of consecutive time increments, a respective location 132 (e.g., 132-1-1) of the upper eyelid;
instructions, programs, data, or information associated with a trace analysis module 134 for:
   obtaining, between a first and second time increment 126 within the plurality of consecutive time increments, a first minimum difference 136 between the location of the upper eyelid 130-1 and the lower eyelid 124-1 of the first eye 122-1 across the time increments in the plurality of consecutive time increments that are between the first and second time increment, and
   obtaining, between the first and second time increment 126 within the plurality of consecutive time increments, a first maximum difference 138 between the location of the upper eyelid 130-1 and the lower eyelid 124-1 of the first eye 122-1 across the time increments in the plurality of consecutive time increments that are between the first and second time increment; and
instructions, programs, data, or information associated with an activation function module 140 that passes the first minimum difference and a difference between the first maximum difference and the first minimum difference through an activation function thereby obtaining a first result used to provide an eye closure status of the first eye 122-1.

In some implementations, one or more of the above-identified elements are stored in one or more of the previously mentioned memory devices and correspond to a set of instructions for performing a function described above. The above-identified modules, data, or programs (e.g., sets of instructions) may not be implemented as separate software programs, procedures, datasets, or modules, and thus various subsets of these modules and data may be combined or otherwise re-arranged in various implementations. In some implementations, the non-persistent memory 111 optionally stores a subset of the modules and data structures identified above. Furthermore, in some embodiments, the memory stores additional modules and data structures not described above. In some embodiments, one or more of the above-identified elements is stored in a computer system, other than that of visualization system 100, that is addressable by visualization system 100 so that visualization system 100 may retrieve all or a portion of such data.

Although FIG. 1 depicts a “system 100,” the figure is intended more as functional description of the various features which may be present in computer systems than as a structural schematic of the implementations described herein. In practice, items shown separately could be combined and some items can be separated. Moreover, although FIG. 1 depicts certain data and modules in non-persistent memory 111, some or all of these data and modules may be in persistent memory 112.

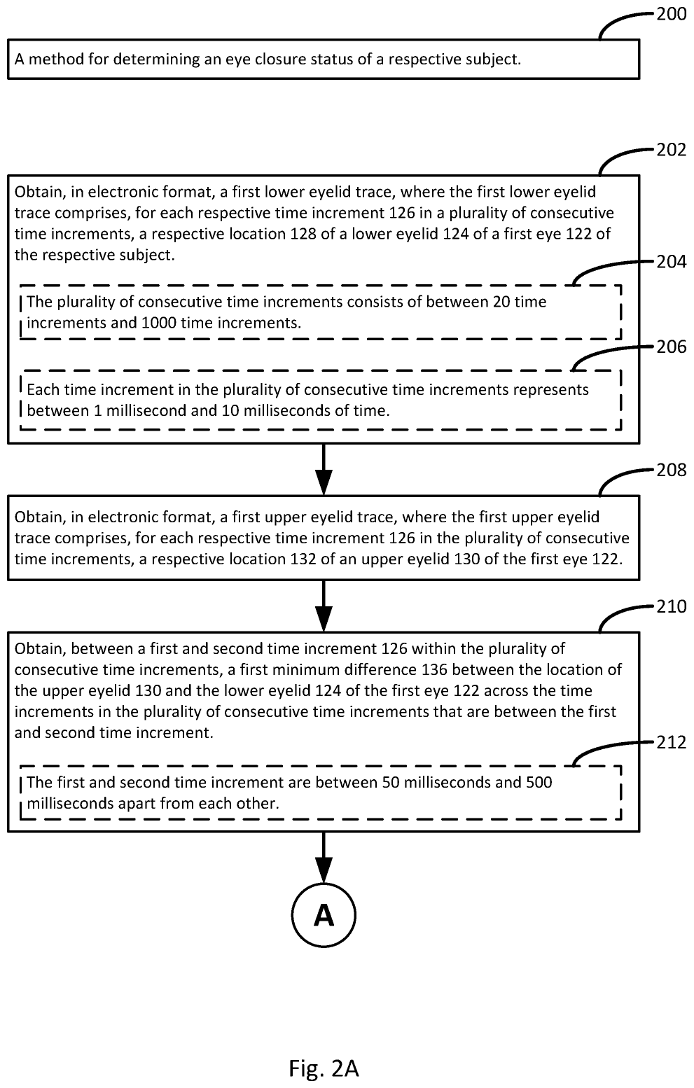

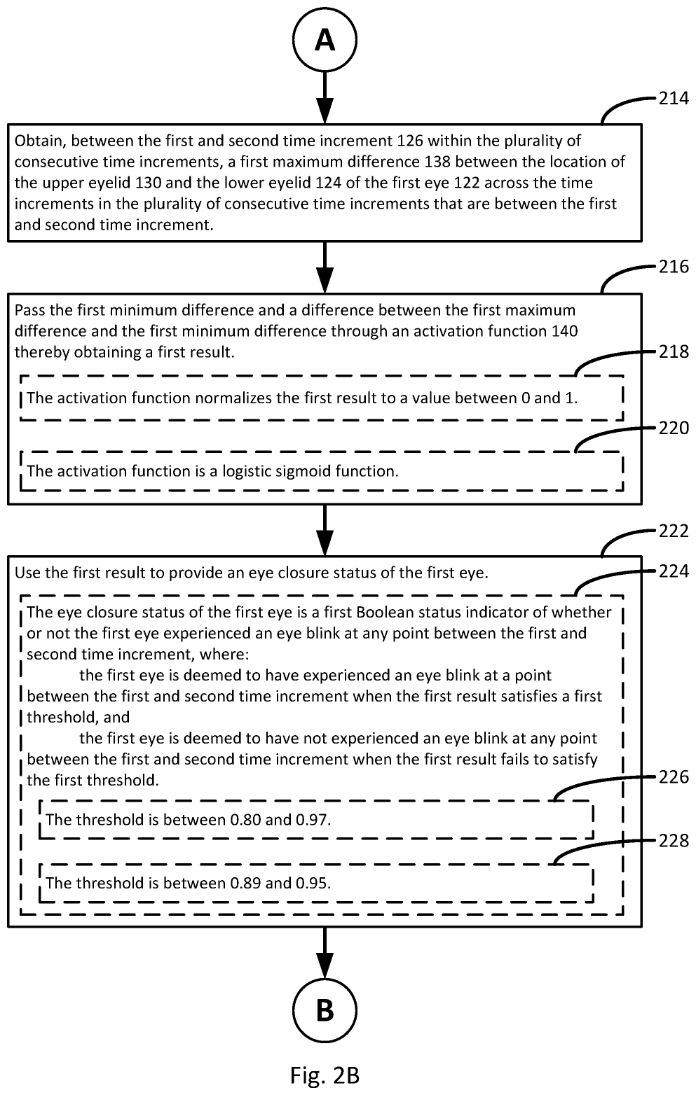

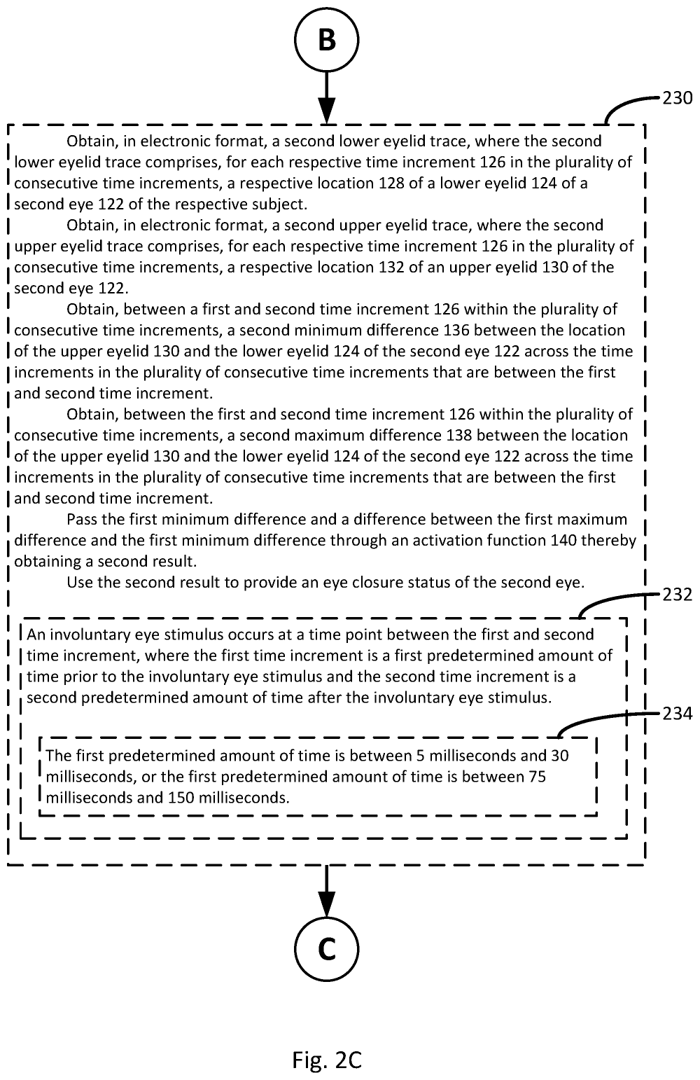

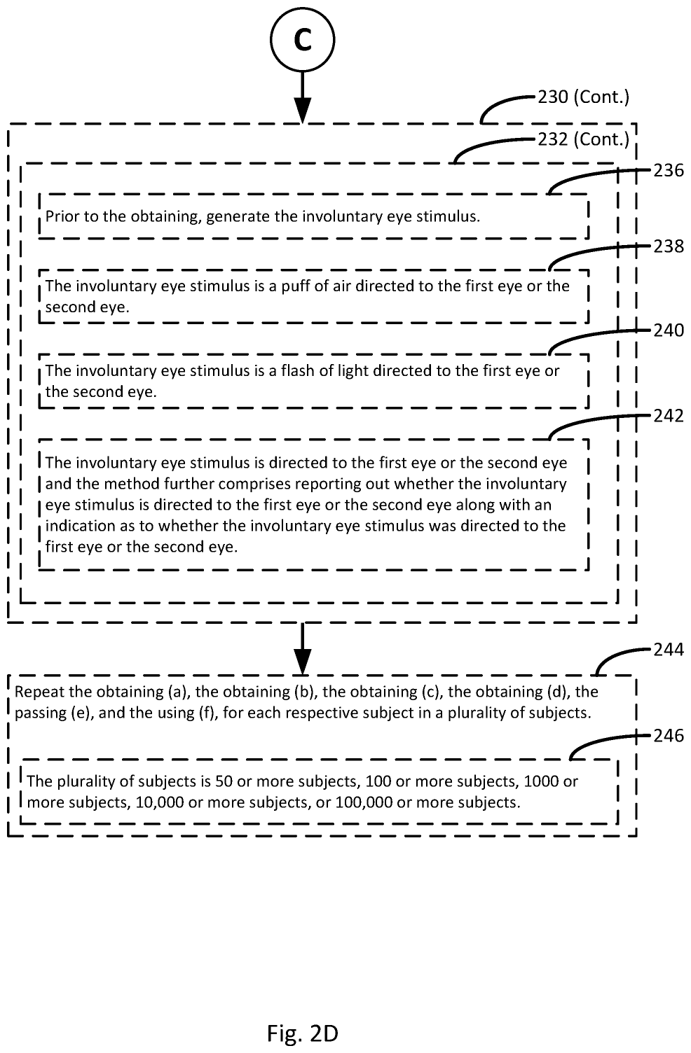

While a system in accordance with the present disclosure has been disclosed with reference to FIG. 1, methods in accordance with the present disclosure are now detailed with reference to FIGS. 2A-D and 3A-B. Any of the disclosed methods can make use of any of the devices, systems or methods disclosed in U.S. patent application Ser. No. 14/787,564, filed May 1, 2014, International Patent Application No. PCT/US2018/032666, having an International Filing Date of May 15, 2018, U.S. Provisional Application No. 63/194,554, filed May 28, 2021, and/or U.S. Provisional Application No. 63/275,749, filed Nov. 4, 2021, each of which is hereby incorporated by reference, in order to determine an eye closure status. For instance, any of the disclosed methods can work in conjunction with any of the disclosed methods or algorithms disclosed in U.S. patent application Ser. No. 14/787,564, filed May 1, 2014, International Patent Application No. PCT/US2018/032666, having an International Filing Date of May 15, 2018, U.S. Provisional Application No. 63/194,554, filed May 28, 2021, and/or U.S. Provisional Application No. 63/275,749, filed Nov. 4, 2021.

### Embodiments for Determining Eye Closure Status.

FIGS. 2A-D and 3A-B collectively illustrate an overview of the techniques in accordance with some embodiments of the present disclosure. In the described embodiments, various methods of determining eye closure status are provided. FIGS. 2A-D collectively illustrate a method 200 for determining an eye closure status of a respective subject.

### Eyelid Traces.

Referring to Block 202, the method includes obtaining, in electronic format, a first lower eyelid trace, where the first lower eyelid trace comprises, for each respective time increment 126 in a plurality of consecutive time increments, a respective location 128 of a lower eyelid 124 of a first eye 122 of the respective subject.

In some embodiments, the first lower eyelid trace is obtained from all or a portion of an image. In some embodiments, the first lower eyelid trace is obtained from all or a portion of a plurality of images. For example, in some embodiments, the first lower eyelid trace is generated from a series of images (e.g., consecutive images) obtained from high-speed image capture. In some embodiments, the first lower eyelid trace is generated from a series of frames (e.g., consecutive frames) obtained from a video.

In some embodiments, a respective image (e.g., frame) used for obtaining the first lower eyelid trace comprises any of the embodiments for images disclosed herein (see, for example, the section entitled “Images,” below).

In some embodiments, each respective time increment in the plurality of consecutive time increments corresponds to a respective image in a high-speed image capture. For example, in some embodiments, each respective time increment in the plurality of consecutive time increments corresponds to a respective frame in a video. In some embodiments, the plurality of consecutive time increments is obtained based on a frame rate (e.g., of a video). Accordingly, in some such embodiments, the plurality of consecutive time increments includes a series of images and/or a plurality of frames in all or a portion of a video. In some embodiments, the plurality of consecutive time increments includes a series of consecutive images obtained from high-speed image capture and/or a plurality of consecutive frames in all or a portion of a video.

In some embodiments, the plurality of consecutive time increments are measured in milliseconds or seconds.

Referring to Block 204, in some embodiments, the plurality of consecutive time increments consists of between 20 time increments and 1000 time increments. In some embodiments, the plurality of consecutive time increments comprises at least 10, at least 20, at least 50, at least 100, at least 200, at least 500, at least 1000, at least 2000, at least 5000, at least 10,000, at least 20,000, or at least 30,000 time increments. In some embodiments, the plurality of consecutive time increments comprises no more than 50,000, no more than 30,000, no more than 10,000, no more than 5000, no more than 1000, no more than 500, or no more than 100 time increments. In some embodiments, the plurality of consecutive time increments comprises from 10 to 500, from 100 to 50,000, from 1000 to 10,000, from 5000 to 15,000, from 200 to 2000, or from 10,000 to 30,000 time increments. In some embodiments, the plurality of consecutive time increments falls within another range starting no lower than 10 time increments and ending no higher than 50,000 time increments.

Referring to Block 206, in some embodiments, each time increment in the plurality of consecutive time increments represents between 1 millisecond (ms) and 10 milliseconds of time. In some embodiments, each time increment in the plurality of consecutive time increments represents at least 0.2, at least 0.5, at least 1, at least 2, at least 3, at least 5, at least 10, at least 20, at least 50, at least 100, at least 200, or at least 500 milliseconds of time. In some embodiments, each time increment in the plurality of consecutive time increments represents no more than 1000, no more than 500, no more than 200, no more than 100, no more than 50, no more than 20, or no more than 10 milliseconds of time. In some embodiments, each time increment in the plurality of consecutive time increments represents from 0.5 to 50, from 1 to 20, from 2 to 10, from 10 to 500, or from 100 to 1000 milliseconds of time. In some embodiments, each time increment in the plurality of consecutive time increments represents another range of time starting no lower than 0.2 milliseconds and ending no higher than 1000 milliseconds.

Accordingly, in some embodiments, the plurality of consecutive time increments corresponds to a total duration of time comprising at least 0.5, at least 1, at least 2, at least 5, at least 10, at least 20, at least 30, at least 40, at least 50, or at least 60 seconds. In some embodiments, the plurality of consecutive time increments corresponds to a total duration of time comprising at least 1, at least 2, at least 3, at least 4, or at least 5 minutes. In some embodiments, the plurality of consecutive time increments corresponds to a total duration of time comprising no more than 10 minutes, no more than 5 minutes, no more than 1 minute, no more than 30 seconds, or no more than 10 seconds. In some embodiments, the plurality of consecutive time increments corresponds to a total duration of time comprising from 1 second to 30 seconds, from 10 seconds to 1 minute, from 30 seconds to 5 minutes, from 2 minutes to 10 minutes, or from 15 seconds to 40 seconds. In some embodiments, the plurality of consecutive time increments corresponds to a total duration of time that falls within another range starting no lower than 0.5 seconds and ending no higher than 10 minutes.

In some embodiments, the first lower eyelid trace comprises, for each respective time increment in the plurality of consecutive time increments, a respective location of the lower eyelid, where the respective location is obtained as a set of coordinates indicating the location of the eyelid in a respective image corresponding to the respective time increment. In some embodiments, the respective location is a set of coordinates that indicates the location of the center of the eyelid. In some embodiments, the respective location is a set of coordinates that indicates an edge of the eyelid (e.g., the upper edge of a bottom eyelid). In some embodiments, the set of coordinates indicates the center of the edge of the eyelid. In some embodiments, a set of coordinates corresponding to the eyelid is measured relative to a reference point. In some such embodiments, the reference point is the top left of the eyelid and/or the top left of the image containing the eyelid (e.g., where the top left of the eyelid and/or the image has the coordinates (0,0)).

In some embodiments, the respective location is obtained using a corresponding “keypoint” for the lower eyelid (see, for instance, the section entitled “Determining eyelid locations,” below).

In some embodiments, a respective location in the first lower eyelid trace comprises any of the embodiments for eyelid locations disclosed herein (see, for example, the section entitled “Determining eyelid locations,” below). In some embodiments, a respective location in the first lower eyelid trace is obtained using any of the embodiments for obtaining eyelid traces and/or eyelid locations disclosed herein (see, for example, the sections entitled “Embodiments for obtaining eyelid traces” and “Determining eyelid locations,” below).

Referring to Block 208, the method further includes obtaining, in electronic format, a first upper eyelid trace, where the first upper eyelid trace comprises, for each respective time increment 126 in the plurality of consecutive time increments, a respective location 132 of an upper eyelid 130 of the first eye 122.

In some embodiments, the first upper eyelid trace includes any of the embodiments disclosed herein as for the first lower eyelid trace. For example, in some embodiments, the first upper eyelid trace is obtained from all or a portion of an image. In some embodiments, the first upper eyelid trace is obtained from all or a portion of a plurality of images. For example, in some embodiments, the first upper eyelid trace is generated from a series of images (e.g., consecutive images) obtained from high-speed image capture. In some embodiments, the first upper eyelid trace is generated from a series of frames (e.g., consecutive frames) obtained from a video.

In some embodiments, a respective image (e.g., frame) used for obtaining the first upper eyelid trace comprises any of the embodiments for images disclosed herein (see, for example, the section entitled “Images,” below).

In some embodiments, the first upper eyelid trace comprises, for each respective time increment in the plurality of consecutive time increments, a respective location of the upper eyelid, where the respective location is obtained as a set of coordinates indicating the location of the eyelid in a respective image corresponding to the respective time increment. In some embodiments, the respective location is a set of coordinates that indicates the location of the center of the eyelid. In some embodiments, the respective location is a set of coordinates that indicates an edge of the eyelid (e.g., the bottom edge of an upper eyelid). In some embodiments, the set of coordinates indicates the center of the edge of the eyelid. In some embodiments, a set of coordinates corresponding to the eyelid is measured relative to a reference point. In some such embodiments, the reference point is the top left of the eyelid and/or the top left of the image containing the eyelid (e.g., where the top left of the eyelid and/or the image has the coordinates (0,0)).

In some embodiments, the respective location is obtained using a corresponding “keypoint” for the upper eyelid (see, for instance, the section entitled “Determining eyelid locations,” below).

In some embodiments, a respective location in the first upper eyelid trace comprises any of the embodiments for eyelid locations disclosed herein (see, for example, the section entitled “Determining eyelid locations,” below). In some embodiments, a respective location in the first upper eyelid trace is obtained using any of the embodiments for obtaining eyelid traces and/or eyelid locations disclosed herein (see, for example, the sections entitled “Embodiments for obtaining eyelid traces” and “Determining eyelid locations,” below).

In some embodiments, the first lower eyelid trace and the first upper eyelid trace each comprise a respective list of eyelid locations for the respective eyelid. In some such embodiments, the first lower eyelid trace is a first list (e.g., vector) containing a plurality of elements, each respective element corresponding to a respective location of the first lower eyelid of the first eye, for each respective time increment in the plurality of consecutive time increments. In some embodiments, the first upper eyelid trace is a second list (e.g., vector) containing a plurality of elements, each respective element corresponding to a respective location of the first upper eyelid of the first eye, for each respective time increment in the plurality of consecutive time increments. Example 3 illustrates, for a respective eye, a lower eyelid trace and an upper eyelid trace, where each respective trace comprises, for the respective eyelid, a list of eyelid locations (see, e.g., Right Top and Right Bottom).

In some embodiments, the method includes determining a difference between the location of the upper eyelid and the location of the lower eyelid, at a respective time increment in the plurality of consecutive time increments. In some embodiments, the difference between the location of the upper eyelid and the location of the lower eyelid represents the distance between (e.g., space between) the upper and the lower eyelids. In some embodiments, the method includes determining a difference between the location of the upper eyelid and the location of the lower eyelid, at each respective time increment in the plurality of consecutive time increments.

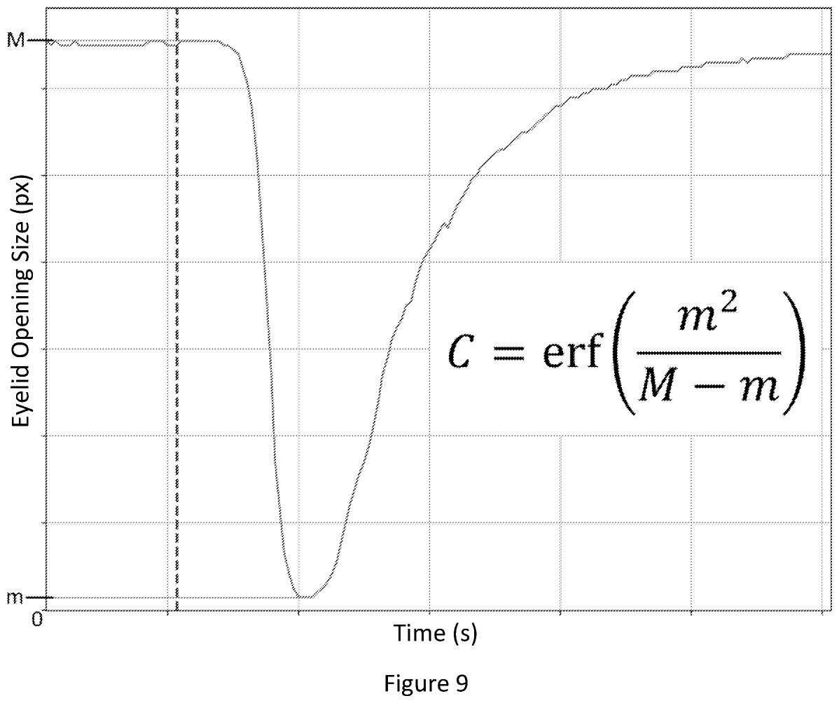

In some embodiments, the method includes determining a difference between the location of the upper eyelid and the location of the lower eyelid, at each respective time increment in a subset of the plurality of consecutive time increments (e.g., a time window). For instance, FIG. 9 illustrates an example diagram plotting the differences between the locations of the upper and lower eyelids in pixels (e.g., “Eyelid Opening Size”), for each respective time increment in a subset of a plurality of consecutive time increments measured in seconds (e.g., “Time”). The solid line indicates the degree of eyelid opening over time, where the maximum difference between the locations of the upper and lower eyelids is marked by capital letter “M” and the minimum difference between the locations of the upper and lower eyelids is marked by lowercase letter “m.”

Accordingly, referring to Block 210, the method includes obtaining, between a first and second time increment 126 within the plurality of consecutive time increments, a first minimum difference 136 between the location of the upper eyelid 130 and the lower eyelid 124 of the first eye 122 across the time increments in the plurality of consecutive time increments that are between the first and second time increment. In some embodiments, the first minimum difference refers to the point at which the upper eyelid and the lower eyelid are closest together during the period between the first and second time increment.

In some embodiments, the first and second time increment are at least 30, at least 40, at least 50, at least 60, at least 70, at least 80, at least 90, at least 100, at least 110, at least 150, at least 175, at least 200, at least 250, at least 300, at least 500, at least 1000, at least 2000, or at least 5000 milliseconds apart from each other. In some embodiments, the first and second time increment are no more than 10,000, no more than 5000, no more than 1000, no more than 500, no more than 200, no more than 100, or no more than 50 milliseconds apart from each other. In some embodiments, the first and second time increment are between 50 and 400, between 100 and 200, between 30 and 1000, between 90 and 120, or between 500 and 5000 milliseconds apart from each other. In some embodiments, the first and second time increment are separated by another duration of time starting no lower than 30 milliseconds and ending no higher than 10,000 milliseconds.

In some embodiments, the set of time increments in the plurality of consecutive time increments that are between the first and second time increment corresponds to a respective time window. In some implementations, a respective time window has a duration including the period of time spanned by the first and second time increment in the plurality of consecutive time increments.

For example, referring to Block 212, in some embodiments, the first and second time increment are between 50 milliseconds and 500 milliseconds apart from each other. In some such embodiments, the time window has a duration of between 50 milliseconds and 500 milliseconds.

In some embodiments, the time window is determined using a known reference time. In some such embodiments, the time window is determined by selecting a first predetermined amount of time prior to the known reference time. In some embodiments, the time window is determined by selecting a second predetermined amount of time after the known reference time. In some embodiments, the time window is determined by selecting a window of time around (e.g., encompassing) the known reference time.

In some embodiments, when the first and/or second predetermined amount of time (e.g., prior to, after, and/or encompassing) the known reference time extends beyond the amount of available time intervals in the plurality of consecutive time intervals, the time window is determined by selecting the largest possible amount of time prior to, after, and/or around the known reference time that can be selected from the plurality of consecutive time intervals. For instance, when the plurality of consecutive time increments encompasses a plurality of consecutive frames in a video, and the first predetermined amount of time prior to the known reference time would extend beyond the first frame of the video, then the time window is determined by selecting all of the frames prior to the reference time up to the first frame of the video, even if the amount of time selected is less than the desired predetermined amount of time.

In some embodiments, a known reference time is a time at which an involuntary eye stimulus is generated. Involuntary eye stimuli are further described below (see, for example, the section entitled “Stimuli,” below).

In some embodiments, the known reference time is a time at which a voluntary blink is performed. In some implementations, the time window is determined by identifying a voluntary blink within an eyelid trace, determining one or more respective time increments in the plurality of consecutive time increments at which the voluntary blink is deemed to occur, assigning a respective time increment in the one or more respective time increments as the known reference time, and selecting a predetermined amount of time prior to and/or after the known reference time at which the voluntary blink occurs.

In some embodiments, the first predetermined amount of time comprises at least 1, at least 2, at least 5, at least 10, at least 15, at least 20, at least 50, at least 100, or at least 200 milliseconds. In some embodiments, the first predetermined amount of time comprises no more than 500, no more than 200, no more than 100, no more than 50, no more than 20, or no more than 10 milliseconds. In some embodiments, the first predetermined amount of time comprises from 1 to 50, from 4 to 20, from 10 to 15, from 20 to 200, or from 100 to 500 milliseconds. In some embodiments, the first predetermined amount of time falls within another range starting no lower than 1 millisecond and ending no higher than 500 milliseconds.

In some embodiments, the second predetermined amount of time comprises at least 20, at least 50, at least 100, at least 200, at least 500, at least 1000, or at least 2000 milliseconds. In some embodiments, the second predetermined amount of time comprises no more than 10,000, no more than 2000, no more than 1000, no more than 500, no more than 200, no more than 100, or no more than 50 milliseconds. In some embodiments, the second predetermined amount of time comprises from 20 to 500, from 50 to 200, from 70 to 150, from 200 to 2000, or from 1000 to 5000 milliseconds. In some embodiments, the second predetermined amount of time falls within another range starting no lower than 20 milliseconds and ending no higher than 10,000 milliseconds.

In some embodiments, the first and second predetermined amount of time comprises any of the embodiments for time windows disclosed herein (as discussed, for example, in the section entitled “Stimuli,” below).

Referring to Block 214, the method further includes obtaining, between the first and second time increment 126 within the plurality of consecutive time increments, a first maximum difference 138 between the location of the upper eyelid 130 and the lower eyelid 124 of the first eye 122 across the time increments in the plurality of consecutive time increments that are between the first and second time increment. In some embodiments, the first maximum difference refers to the point at which the upper eyelid and the lower eyelid are farthest apart during the period between the first and second time increment.

In some embodiments, the first minimum difference and the first maximum difference between the locations of the upper eyelid and the lower eyelid of the first eye are measured in pixels. In some embodiments, the first minimum difference and the first maximum difference between the locations of the upper eyelid and the lower eyelid of the first eye are measured in any unit of distance that can be applied to an image of an eye (e.g., centimeters, millimeters, etc.).

In some embodiments, prior to obtaining the first minimum difference and the first maximum difference between the locations of the upper eyelid and the lower eyelid of the first eye, the method includes adjusting the locations of the upper eyelid in the first upper eyelid trace and/or the locations of the lower eyelid in the first lower eyelid trace. For instance, in some implementations, one or more locations of the lower eyelid in the first lower eyelid trace are adjusted to zero.

In some embodiments, the method does not include obtaining the first lower eyelid trace, and, for each respective time interval in the plurality of consecutive time intervals, the location of the lower eyelid is assigned a value of zero. In some such embodiments, the obtaining the first minimum difference and the first maximum difference between the locations of the upper eyelid and the lower eyelid of the first eye is based on the respective location of the upper eyelid in the first upper eyelid trace, for each respective time increment in the plurality of consecutive time increments.

### Eye Closure Results.

In some embodiments, the method includes obtaining an eye closure score (e.g., a first result) using the first minimum difference and the first maximum difference between the locations of the upper eyelid and the lower eyelid of the first eye. The eye closure score is used to determine the eye closure status of the first eye.

In some embodiments, the method includes performing a normalization upon a range formed from the first minimum difference and the first maximum difference. For example, in some implementations, the normalization transforms the range between the first minimum difference and the first maximum difference (e.g., between degrees of eye closures, where the maximum difference denotes eyes that are open, and the minimum difference denotes eyes that are closed or nearly closed) from a first interval to a second interval. In some such embodiments, the normalization results in the first minimum difference and the first maximum difference mapped onto a smaller interval. In some embodiments, the normalization results in the first minimum difference and the first maximum difference mapped onto a larger interval.

Accordingly, referring to Block 216, in some embodiments, the method includes passing the first minimum difference and a difference between the first maximum difference and the first minimum difference through an activation function 140 thereby obtaining a first result. In some implementations, the first maximum difference and the first minimum difference form a range that is of a first interval, and the activation function transforms the range such that the first result is constrained within an upper bound and a lower bound of a second interval.

In some embodiments, the activation function is a sigmoid function. In some embodiments, the activation function is a logistic function, or a logit function.

In some embodiments, the activation function is an ReLU function, a Softmax function, or a tanh function. In some embodiments, the activation function is selected from the group consisting of: tanh, sigmoid, softmax, Gaussian, Boltzmann-weighted averaging, absolute value, linear, rectified linear unit (ReLU), bounded rectified linear, soft rectified linear, parameterized rectified linear, average, max, min, sign, square, square root, multiquadric, inverse quadratic, inverse multiquadric, polyharmonic spline, swish, mish, Gaussian error linear unit (GeLU), scaled exponential linear unit (SELU), and/or thin plate spline. Non-limiting examples of activation functions contemplated for use in the present disclosure are further described, for example, in the section entitled “Definitions: Classifiers,” above.

In some embodiments, the activation function evaluates the first minimum difference and the difference between the first maximum difference and the first minimum difference as an Nth power of (i) the first minimum difference divided by (ii) the difference between the first maximum difference and the first minimum difference.

In some embodiments, N is a positive integer of 2 or greater. In some embodiments, N is a positive integer of at least 1, at least 2, at least 3, at least 4, at least 5, at least 6, at least 7, at least 8, at least 9, or at least 10. In some embodiments, N is a positive integer of no more than 20, no more than 10, no more than 5, no more than 3, or no more than 2. In some embodiments, N is a positive integer from 1 to 5, from 2 to 10, from 3 to 15, or from 8 to 20. In some embodiments, N falls within another range starting no lower than 1 and ending no higher than 20.

In some embodiments, N is 2.

In some embodiments, the method includes passing a square of the first minimum difference divided by a difference between the first maximum difference and the first minimum difference through a sigmoid function thereby obtaining a first result. FIG. 9 illustrates an example of the first result (e.g., “C”), which is obtained by dividing the square of the first minimum difference (e.g., “m”) by the difference between the first maximum difference and the first minimum difference (e.g., “M−m”) and passing the outcome through a sigmoid function (e.g., “erf”).

Generally, the sigmoid function serves to transform the range between degrees of eye closures (e.g., where the maximum difference denotes eyes that are open, and the minimum difference denotes eyes that are closed or nearly closed) such that the first result is constrained within upper and lower bounds. The first result is therefore normalized to a scale that can be systematically applied across multiple analyses (e.g., a plurality of different time windows, different consecutive time intervals, different blinks, different eyes, and/or different subjects).

Referring to Block 218, in some embodiments, the activation function normalizes the first result to a value between 0 and 1. In some embodiments, the activation function normalizes the first result to a value between −1 and 1.

Any suitable sigmoid function is contemplated for use in the present disclosure, as will be apparent to one skilled in the art. For instance, in some embodiments, the sigmoid function is a hyperbolic tangent function, an arctangent function, a Gudermannian function, an error function, a generalized logistic function, a smoothstep function and/or an algebraic function. Referring to Block 220, in some embodiments, the sigmoid function is a logistic sigmoid function.

In some embodiments, the activation function is a logistic or a linear regression function. In some embodiments, the activation function is a regression algorithm, including but not limited to logistic regression with lasso, L2 or elastic net regularization. In some embodiments, the regression algorithm is any of the regression algorithms disclosed herein (see, for example, the section entitled “Definitions: Classifiers,” above).

In some embodiments, the method further comprises, when the difference between the first maximum difference and the first minimum difference is zero, adjusting the difference between the first maximum difference and the first minimum difference to be a non-zero positive value. In some such embodiments, the non-zero positive value is 1.

As described above, referring to Block 222, the method further includes using the first result to provide an eye closure status of the first eye.

For instance, in some embodiments, the first result represents a degree of eye closure between the upper and lower eyelids. In some embodiments, the first result is a number of pixels that represents a distance between the upper and lower eyelids. In some embodiments, the first result is applied to a first threshold to determine whether the degree of eye closure between the upper and lower eyelids (e.g., the number of pixels that represents a distance between the upper and lower eyelids at the point of minimum difference) can be classified as a blink.

In some embodiments, the eye closure status of the first eye is a Boolean status indicator (e.g., closed or open, blink or not blink, true or false, etc.).

Accordingly, referring to Block 224, in some embodiments, the eye closure status of the first eye is a first Boolean status indicator of whether or not the first eye experienced an eye blink at any point between the first and second time increment, where the first eye is deemed to have experienced an eye blink at a point between the first and second time increment when the first result satisfies a first threshold, and the first eye is deemed to have not experienced an eye blink at any point between the first and second time increment when the first result fails to satisfy the first threshold.

In some implementations, when the first result is less than the threshold, then the first eye is deemed to have experienced an eye blink (indicating that the eyelids have been brought together at a close enough distance to be considered “closed” or “blink”). In some implementations, when the first result is greater than or equal to the threshold, then the first eye is deemed not to have experienced an eye blink (indicating that the eyelids have not closed sufficiently to be considered a “blink”). In some implementations, when the first result is less than or equal to the threshold, the first eye is deemed to have experienced an eye blink, and when the first result is greater than the threshold, the first eye is deemed not to have experienced an eye blink.

In some implementations, when the first result is greater than the threshold, then the first eye is deemed to have experienced an eye blink. In some implementations, when the first result is less than or equal to the threshold, then the first eye is deemed not to have experienced an eye blink. In some implementations, when the first result is greater than or equal to the threshold, the first eye is deemed to have experienced an eye blink, and when the first result is less than the threshold, the first eye is deemed not to have experienced an eye blink.

Referring to Block 226, in some such embodiments, the threshold is between 0.80 and 0.97. Referring to Block 228, in some such embodiments, the threshold is between 0.89 and 0.95. In some embodiments, the threshold is at least 0.5, at least 0.6, at least 0.7, at least 0.8, at least 0.85, at least 0.9, at least 0.91, at least 0.92, at least 0.93, at least 0.94, or at least 0.95. In some embodiments, the threshold is no more than 0.99, no more than 0.98, no more than 0.97, no more than 0.96, no more than 0.95, no more than 0.9, no more than 0.8, or no more than 0.7. In some embodiments, the threshold is from 0.5 to 0.95, from 0.6 to 0.98, or from 0.9 to 0.94. In some embodiments, the threshold falls within another range starting no lower than 0.5 and ending no higher than 0.99. Advantageously, application of a threshold to the first result allows for the detection of an eye blink even in cases where the distance between the upper and lower eyelids is greater than 0 (e.g., where the eye is not fully closed), as described above.

In some embodiments, the method includes reporting out the closure status of the first eye. In some such embodiments, the method comprises reporting out whether the first eye has experienced a blink. For instance, in some implementations, the closure status is reported as “TRUE” when the first eye was deemed to have experienced a blink, and the closure status is reported as “FALSE” when the first eye was deemed not to have experienced a blink. In some implementations, the closure status is reported as “1” when the first eye was deemed to have experienced a blink, and the closure status is reported as “0” when the first eye was deemed not to have experienced a blink. In some implementations, the closure status is reported as “blink” when the first eye was deemed to have experienced a blink, and the closure status is reported as “no blink” when the first eye was deemed not to have experienced a blink.

In some embodiments, the eye closure status of the first eye is a degree of closure. In some such embodiments, the degree of closure is a value between 0 and 1 indicating the degree to which the eye closed, where a value of 0 indicates full closure and a value of 1 means the eye had no closure response.

In some embodiments, the eye closure status of the first eye is a degree of closure at any point between the first and second time increment, where the first eye is deemed to have experienced a first degree of closure at a point between the first and second time increment when the first result satisfies a first threshold, the first eye is deemed to have experienced a second degree of closure at a point between the first and second time increment when the first result satisfies a second threshold.

For instance, in some such embodiments, when the first result is less than a first threshold, then the first eye is deemed to be closed, when the first result is greater than or equal to the first threshold but less than a second threshold, then the first eye is deemed to be partially open, and when the first result is greater than or equal to the second threshold, then the first eye is deemed to be open.

Other alternative embodiments are possible, including any number of thresholds to indicate any corresponding number of degrees of closure, as will be apparent to one skilled in the art. For instance, in some embodiments, the method includes applying at least 1, at least 2, at least 3, at least 4, at least 5, at least 6, or at least 7 or more thresholds to the first result, to indicate a corresponding at least 1, at least 2, at least 3, at least 4, at least 5, at least 6, or at least 7 or more degrees of closure.

In some embodiments, the method includes performing the determination of eye closure status for a plurality of eyes of the respective subject. Thus, referring to Block 230, in some embodiments, the method further comprises obtaining, in electronic format, a second lower eyelid trace, where the second lower eyelid trace comprises, for each respective time increment 126 in the plurality of consecutive time increments, a respective location 128 of a lower eyelid 124 of a second eye 122 of the respective subject. The method includes obtaining, in electronic format, a second upper eyelid trace, where the second upper eyelid trace comprises, for each respective time increment 126 in the plurality of consecutive time increments, a respective location 132 of an upper eyelid 130 of the second eye 122.

The method further includes obtaining, between a first and second time increment 126 within the plurality of consecutive time increments, a second minimum difference 136 between the location of the upper eyelid 130 and the lower eyelid 124 of the second eye 122 across the time increments in the plurality of consecutive time increments that are between the first and second time increment. Additionally, the method includes obtaining, between the first and second time increment 126 within the plurality of consecutive time increments, a second maximum difference 138 between the location of the upper eyelid 130 and the lower eyelid 124 of the second eye 122 across the time increments in the plurality of consecutive time increments that are between the first and second time increment. A square of the second minimum difference divided by a difference between the second maximum difference and the second minimum difference is passed through an activation function 140 thereby obtaining a second result. The second result is used to provide an eye closure status of the second eye.

Any suitable methods and/or embodiments disclosed herein with respect to determining eye closure status for the first eye are contemplated for use in determining eye closure status for the second eye, as will be apparent to one skilled in the art. For instance, any of the methods and/or embodiments described in the sections entitled “Eyelid traces” and “Eye closure results,” above, are contemplated for use in determining eye closure status for the second eye.

In some embodiments, the first eye is a left eye of the respective subject and the second eye is a right eye of the respective subject. In some embodiments, the first eye is a right eye of the respective subject and the second eye is a left eye of the respective subject.

In some embodiments, the first lower eyelid trace, the first upper eyelid trace for the first eye, the second lower eyelid trace, and the second upper eyelid trace for the second eye each comprise a respective list of eyelid locations, for each respective eyelid, for each respective eye. In some such embodiments, the second lower eyelid trace is a third list (e.g., vector) containing a plurality of elements, each respective element corresponding to a respective location of the second lower eyelid of the second eye, for each respective time increment in the plurality of consecutive time increments. In some embodiments, the second upper eyelid trace is a fourth list (e.g., vector) containing a plurality of elements, each respective element corresponding to a respective location of the second upper eyelid of the second eye, for each respective time increment in the plurality of consecutive time increments. Example 3 illustrates, for each respective eye in a plurality of eyes including a left eye and a right eye, a lower eyelid trace and an upper eyelid trace, where each respective trace comprises, for the respective eyelid, a list of eyelid locations (see, e.g., Right Top, Right Bottom, Left Top, Left Bottom).

### Stimuli.

In some embodiments, the method comprises generating an involuntary eye stimulus. In some embodiments, the involuntary eye stimulus is applied to a left eye or a right eye of the respective subject. In some embodiments, the involuntary eye stimulus is applied to both eyes of the respective subject.

Referring to Block 232, in some embodiments, an involuntary eye stimulus occurs at a time point between the first and second time increment, where the first time increment is a first predetermined amount of time prior to the involuntary eye stimulus and the second time increment is a second predetermined amount of time after the involuntary eye stimulus.

Thus, in some embodiments, a respective time window (e.g., within which a respective minimum difference and/or a respective maximum difference between the locations of the upper and lower eyelids of a respective eye are obtained) includes an involuntary eye stimulus. In some embodiments, a respective time window is determined based upon the occurrence of an involuntary eye stimulus within the plurality of consecutive time increments. The example diagram in FIG. 9 illustrates the occurrence of an involuntary eye stimulus (e.g., a puff of air directed to the respective eye) as a dashed vertical line, within the subset of the plurality of consecutive time increments. The solid line indicating the degree of eyelid opening over time can thus be visualized relative to the time at which the involuntary eye stimulus occurred.

Referring to Block 234, the first predetermined amount of time is between 5 milliseconds and 30 milliseconds, and the second predetermined amount of time is between 75 milliseconds and 150 milliseconds.

In some embodiments, the first predetermined amount of time comprises at least 1, at least 2, at least 5, at least 10, at least 15, at least 20, at least 50, at least 100, or at least 200 milliseconds. In some embodiments, the first predetermined amount of time comprises no more than 500, no more than 200, no more than 100, no more than 50, no more than 20, or no more than 10 milliseconds. In some embodiments, the first predetermined amount of time comprises from 1 to 50, from 4 to 20, from 10 to 15, from 20 to 200, or from 100 to 500 milliseconds. In some embodiments, the first predetermined amount of time falls within another range starting no lower than 1 millisecond and ending no higher than 500 milliseconds.

In some embodiments, the second predetermined amount of time comprises at least 20, at least 50, at least 100, at least 200, at least 500, at least 1000, or at least 2000 milliseconds. In some embodiments, the second predetermined amount of time comprises no more than 10,000, no more than 2000, no more than 1000, no more than 500, no more than 200, no more than 100, or no more than 50 milliseconds. In some embodiments, the second predetermined amount of time comprises from 20 to 500, from 50 to 200, from 70 to 150, from 200 to 2000, or from 1000 to 5000 milliseconds. In some embodiments, the second predetermined amount of time falls within another range starting no lower than 20 milliseconds and ending no higher than 10,000 milliseconds.

In some embodiments, the first and second predetermined amount of time comprises any of the embodiments for time windows disclosed herein (as discussed, for example, in the section entitled “Eyelid traces,” above).

Referring to Block 236, in some embodiments, the method further comprises, prior to the obtaining the first lower eyelid trace (and/or the first upper eyelid trace), generating the involuntary eye stimulus. In some embodiments, the method further comprises, prior to the obtaining the second lower eyelid trace and/or the second upper eyelid trace, generating the involuntary eye stimulus.

In some embodiments, the involuntary eye stimulus stimulates at least one facial region of the respective subject so as to cause an involuntary blink response in the subject. In some embodiments, the at least one facial region is selected from the group consisting of the temple, the outer canthus, and the eye. In some embodiments, the stimulation of the at least one facial region is provided in proximity of the left eye, the right eye, or both. In some embodiments, the plurality of images is taken for the left eye, the right eye, or both.

In some implementations, the stimulation of the at least one facial region is selected from the group consisting of: a puff of fluid (e.g., the fluid corresponding to a gas, a liquid, or a vapor); a mechanical contact (e.g., a pin prick); one or more flashes of light; an electrical current (e.g., less than approximately 1 milliamp); or a sound (e.g., between 70 and 80 decibels). In some implementations, the stimulation is a verbal or visual command. For example, in some embodiments, the stimulation is a verbal command (e.g., “blink”) administered to the subject. In some embodiments, the stimulation is a visual command (e.g., a screen that displays the word “blink”) that is presented to the subject.

For instance, referring to Block 238, in some embodiments, the involuntary eye stimulus is a puff of air directed to the first eye or the second eye. Referring to Block 240, in some embodiments, the involuntary eye stimulus is a flash of light directed to the first eye or the second eye.

Methods for generating involuntary eye stimuli and stimulating blink reflexes are disclosed in further detail in U.S. patent application Ser. No. 14/787,564, filed May 1, 2014, International Patent Application No. PCT/US2018/032666, having an International Filing Date of May 15, 2018, U.S. Provisional Application No. 63/194,554, filed May 28, 2021, and/or U.S. Provisional Application No. 63/275,749, filed Nov. 4, 2021, each of which is hereby incorporated herein by reference in its entirety for all purposes.

In some embodiments, the method includes generating an involuntary eye stimulus applied to at least one facial region of a respective subject and obtaining an eyelid trace (upper and/or lower) for the ipsilateral eye (e.g., the eye on the same side as the involuntary eye stimulus). For instance, in some embodiments, the method includes generating an involuntary eye stimulus applied to a first eye of a subject and obtaining an eyelid trace (upper and/or lower) for the first eye. In some such embodiments, a stimulus is applied to a left eye and an eyelid trace is obtained for the left eye. In some embodiments, a stimulus is applied to a right eye and an eyelid trace is obtained for the right eye.

In some embodiments, the method includes generating an involuntary eye stimulus applied to at least one facial region of a respective subject and obtaining an eyelid trace (upper and/or lower) for the contralateral eye (e.g., the eye on the opposite side as the involuntary eye stimulus). For instance, in some embodiments, the method includes generating an involuntary eye stimulus applied to a first eye of a subject and obtaining an eyelid trace (upper and/or lower) for a second eye of the subject. In some such embodiments, a stimulus is applied to a left eye and an eyelid trace is obtained for the right eye. In some embodiments, a stimulus is applied to a right eye and an eyelid trace is obtained for the left eye.

Accordingly, the method can be performed as described herein for either eye, whether stimulated or unstimulated (e.g., ipsilateral and/or contralateral to an involuntary eye stimulus).

Referring to Block 242, in some embodiments, the involuntary eye stimulus is directed to the first eye or the second eye and the method further comprises reporting out whether the involuntary eye stimulus is directed to the first eye or the second eye along with an indication as to whether the involuntary eye stimulus was directed to the first eye or the second eye.

Thus, in some embodiments, the method further comprises reporting out whether the eye closure status of the first eye is ipsilateral to the stimulus. In some embodiments, the method further comprises reporting out whether the eye closure status of the first eye is contralateral to the stimulus. In some embodiments, the method further comprises reporting out whether the eye closure status of the second eye is ipsilateral to the stimulus. In some embodiments, the method further comprises reporting out whether the eye closure status of the second eye is contralateral to the stimulus. Example 3 provides an illustrative report including, for each respective involuntary eye stimulus in a plurality of involuntary eye stimuli, an indication of the stimulated side (e.g., left or right) and an eye closure status for each respective eye (e.g., ipsilateral and contralateral) responsive to the respective involuntary eye stimulus.

In some embodiments, the method includes reporting out the type of stimulus used (e.g., a puff of air, a flash of light, an audio prompt, etc.).

In some embodiments, the method comprises generating a plurality of involuntary eye stimuli. In some implementations, each respective stimulus in a plurality of involuntary eye stimuli comprises any of the methods and/or embodiments for stimuli disclosed herein, as will be apparent to one skilled in the art.

In some embodiments, the plurality of involuntary eye stimuli comprises at least 3, at least 5, at least 10, at least 20, at least 30, at least 50, at least 80, at least 100, or at least 150 stimuli. In some embodiments, the plurality of involuntary eye stimuli comprises no more than 200, no more than 150, no more than 100, no more than 50, no more than 30, no more than 20, or no more than 10 stimuli. In some embodiments, the plurality of involuntary eye stimuli comprises from 3 to 50, from 5 to 20, from 6 to 12, from 40 to 100, or from 30 to 200 stimuli. In some embodiments, the plurality of involuntary eye stimuli falls within another range starting no lower than 3 stimuli and ending no higher than 200 stimuli.

In some embodiments, a first respective stimulus and a second respective stimulus in the plurality of stimuli are directed to the same eye or to different eyes. In some embodiments, a first respective stimulus and a second respective stimulus in the plurality of stimuli comprises the same type or different types of stimuli (e.g., a puff of air and a flash of light).

### Batch Processing.

In some embodiments, the presently disclosed systems and methods are used to determine a respective eye closure status for each respective time window in a plurality of time windows.

For instance, in some embodiments, the method further comprises, after the (a) obtaining the first lower eyelid trace and the (b) obtaining the first upper eyelid trace, repeating the (c) obtaining the first minimum difference, the (d) obtaining the first maximum difference, the (e) passing, and the (f) using to provide an eye closure status of a first eye for a respective subject at each respective time window in a plurality of time windows.

In some embodiments, each respective time window in a plurality of time windows includes a set of time increments in the plurality of consecutive time increments that are between a respective first time increment and a respective second time increment corresponding to the respective time window.

In some embodiments, a respective time window corresponds to a respective eye blink (e.g., in a plurality of eye blinks). In some embodiments, a respective time window corresponds to a respective voluntary blink. In some embodiments, a respective time window corresponds to a respective involuntary blink.

In some embodiments, a respective time window corresponds to a respective involuntary eye stimulus. For instance, in some embodiments, each respective time window corresponds to a respective involuntary eye stimulus in a plurality of involuntary eye stimuli. As illustrated in Example 3 and in Table 1, below, in some embodiments, the method comprises generating a plurality of involuntary eye stimuli such that a respective eyelid trace for one or more eyes of a respective subject includes a plurality of consecutive time increments that spans the plurality of involuntary eye stimuli. For each respective involuntary eye stimulus, the method includes (i) generating a respective time window that includes a first predetermined amount of time prior to the involuntary eye stimulus and a second predetermined amount of time after the involuntary eye stimulus and (ii) determining an eye closure status of the one or more eyes of the respective subject within the respective time window (e.g., responsive to the stimulus), using any of the methods and/or embodiments disclosed herein.

In some embodiments, the plurality of time windows comprises at least 3, at least 5, at least 10, at least 20, at least 30, at least 50, at least 80, at least 100, or at least 150 time windows. In some embodiments, the plurality of time windows comprises no more than 200, no more than 150, no more than 100, no more than 50, no more than 30, no more than 20, or no more than 10 time windows. In some embodiments, the plurality of time windows comprises from 3 to 50, from 5 to 20, from 6 to 12, from 40 to 100, or from 30 to 200 time windows. In some embodiments, the plurality of time windows falls within another range starting no lower than 3 time windows and ending no higher than 200 time windows.

In some embodiments, each respective time window in the plurality of time windows corresponds to a respective involuntary eye stimulus in a plurality of involuntary eye stimuli, where the plurality of involuntary eye stimuli comprises at least 3, at least 5, at least 10, at least 20, at least 30, at least 50, at least 80, at least 100, or at least 150 stimuli. In some embodiments, the plurality of involuntary eye stimuli comprises no more than 200, no more than 150, no more than 100, no more than 50, no more than 30, no more than 20, or no more than 10 stimuli. In some embodiments, the plurality of involuntary eye stimuli comprises from 3 to 50, from 5 to 20, from 6 to 12, from 40 to 100, or from 30 to 200 stimuli. In some embodiments, the plurality of involuntary eye stimuli falls within another range starting no lower than 3 stimuli and ending no higher than 200 stimuli.

In some embodiments, for each respective time window in a plurality of time windows, the method is repeated for the first eye and/or for the second eye.

In some embodiments, the method comprises repeating, for each respective time window in the plurality of time windows, any of the methods and/or embodiments disclosed herein, or any substitutions, modifications, additions, deletions, and/or combinations thereof, as will be apparent to one skilled in the art.

In some embodiments, the presently disclosed systems and methods are used to determine a respective eye closure status for each respective instance in a plurality of instances. In some embodiments, a respective instance is a scan of a respective subject (e.g., a respective series of images obtained from high-speed image capture and/or a respective video). In some embodiments, a plurality of instances is obtained for a respective subject when the same subject is used to obtain eyelid traces on a number of different occasions. For instance, applications in which multiple instances are obtained for a respective subject include, but are not limited to, longitudinal studies in which a respective subject is monitored over time, assessment of changes in neurological function and/or blink reflex, diagnosing or determining progression of disease, evaluating treatment effectiveness and/or detecting impairment after traumatic head injury.

In some embodiments, the method further comprises repeating the (a) obtaining the first lower eyelid trace, the (b) obtaining the first upper eyelid trace, the (c) obtaining the first minimum difference, the (d) obtaining the first maximum difference, the (e) passing, and the (f) using to provide an eye closure status of a first eye for a respective subject, for each respective instance in a plurality of instances.

In some embodiments, the plurality of instances comprises at least 3, at least 5, at least 10, at least 20, at least 30, at least 50, at least 80, at least 100, or at least 150 instances. In some embodiments, the plurality of instances comprises no more than 200, no more than 150, no more than 100, no more than 50, no more than 30, no more than 20, or no more than 10 instances. In some embodiments, the plurality of instances comprises from 3 to 50, from 5 to 20, from 6 to 12, from 40 to 100, or from 30 to 200 instances. In some embodiments, the plurality of instances falls within another range starting no lower than 3 instances and ending no higher than 200 instances.

In some embodiments, for each respective instance in a plurality of instances, the method is repeated for the first eye and/or for the second eye.

In some embodiments, the method comprises repeating, for each respective instance in the plurality of instances, any of the methods and/or embodiments disclosed herein, or any substitutions, modifications, additions, deletions, and/or combinations thereof, as will be apparent to one skilled in the art.

In some embodiments, the presently disclosed systems and methods are used to determine a respective eye closure status for each respective subject in a plurality of subjects. In some embodiments, each respective subject in the plurality of subjects corresponds to a training object in a plurality of training objects in a training dataset. For instance, in some embodiments, each respective subject in the plurality of subjects is used to train a model (e.g., a neural network) to detect eye closure status. In some such embodiments, the method comprises determining a respective eye closure status for each respective subject in a plurality of subjects and inputting to a model (e.g., a neural network) at least the respective eye closure status for each respective subject in the plurality of subjects. In some embodiments, each respective subject in the plurality of subjects is a patient in a plurality of patients (e.g., in a clinical trial).

Accordingly, referring to Block 244, in some embodiments, the method further comprises repeating the obtaining (a), the obtaining (b), the obtaining (c), the obtaining (d), the passing (e), and the using (f), for each respective subject in a plurality of subjects.

Referring to Block 246, in some embodiments, the plurality of subjects is 50 or more subjects, 100 or more subjects, 1000 or more subjects, 10,000 or more subjects, or 100,000 or more subjects. In some embodiments, the plurality of subjects comprises at least 20, at least 50, at least 100, at least 200, at least 500, at least 1000, at least 5000, at least 10,000, at least 20,000, at least 50,000, at least 100,000, or at least 500,000 subjects. In some embodiments, the plurality of subjects comprises no more than 1 million, no more than 500,000, no more than 100,000, no more than 50,000, no more than 10,000, no more than 5000, no more than 1000, or no more than 500 subjects. In some embodiments, the plurality of subjects comprises from 100 to 1000, from 500 to 10,000, from 200 to 20,000, from 5000 to 100,000, or from 100,000 to 500,000 subjects. In some embodiments, the plurality of subjects falls within another range starting no lower than 20 subjects and ending no higher than 1 million subjects.

In some embodiments, the method comprises repeating, for each respective subject in the plurality of subjects, any of the methods and/or embodiments disclosed herein, or any substitutions, modifications, additions, deletions, and/or combinations thereof, as will be apparent to one skilled in the art. Moreover, any combination of one or more eyes, one or more time windows, one or more instances, and/or one or more subjects, as described in the foregoing sections, is contemplated for use in the presently disclosed systems and methods for determining eye closure status.

### Embodiments for Obtaining Eyelid Traces.

In some embodiments, as described above, a respective eyelid trace (e.g., upper and/or lower) is generated by, for each respective time increment in the plurality of consecutive time increments, obtaining the respective location of the respective eyelid as a set of coordinates indicating the location of the eyelid in a respective image corresponding to the respective time increment. In some such embodiments, eyelid locations are obtained using eyelid keypoints.

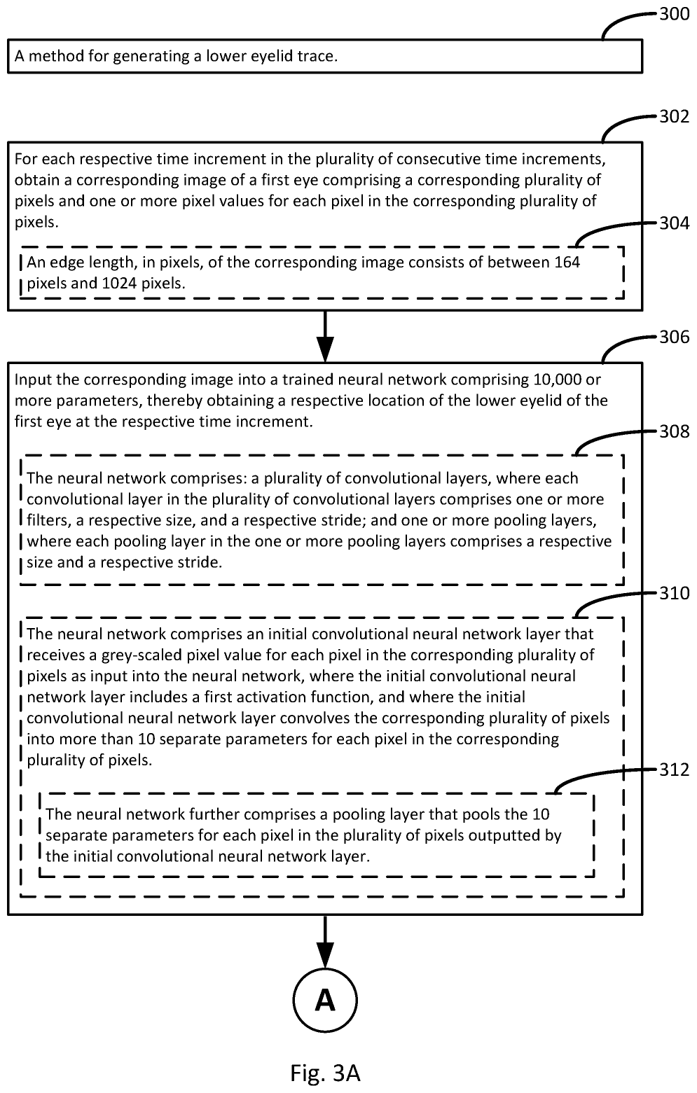

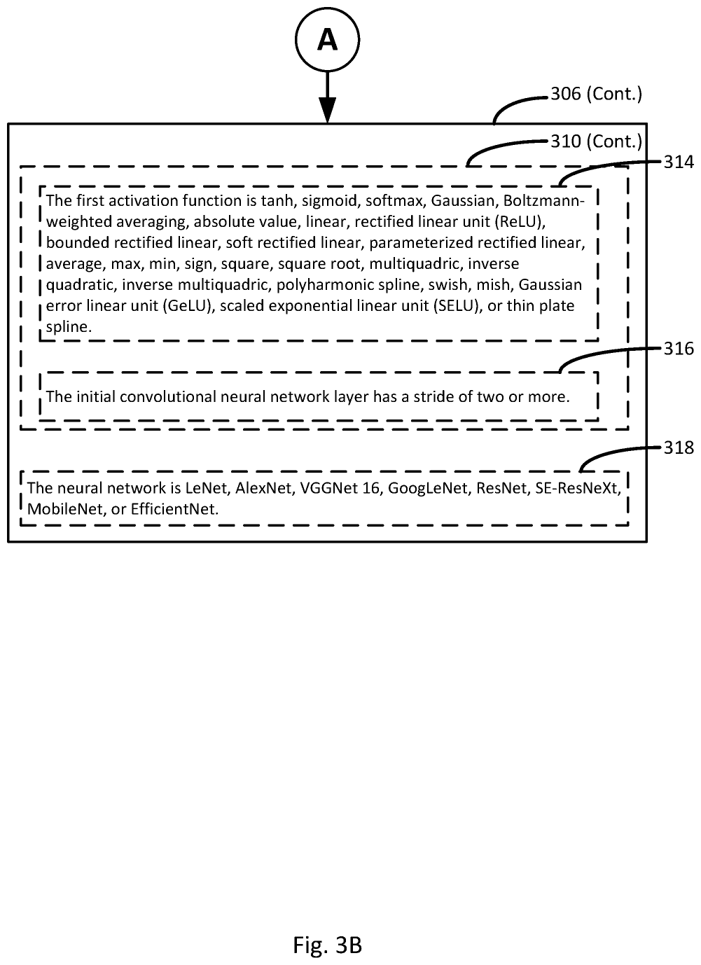

FIGS. 3A-B collectively illustrate a method 300 for generating a respective eyelid trace for a respective subject. In some embodiments, the method includes generating a respective lower eyelid trace by a procedure comprising, for each respective time increment in the plurality of consecutive time increments, (i) obtaining a corresponding image of a respective eye comprising a corresponding plurality of pixels and one or more pixel values for each pixel in the corresponding plurality of pixels, and (ii) inputting the corresponding image into a trained neural network comprising a plurality of parameters (e.g., 10,000 or more parameters), thereby obtaining the respective location of the lower eyelid of the respective eye at the respective time increment. In some embodiments, the method further includes generating a first upper eyelid trace by a procedure comprising, for each respective time increment in the plurality of consecutive time increments, (i) obtaining a corresponding image of a respective eye comprising a corresponding plurality of pixels and one or more pixel values for each pixel in the corresponding plurality of pixels, and (ii) inputting the corresponding image into a trained neural network comprising a plurality of parameters (e.g., 10,000 or more parameters), thereby obtaining the respective location of the upper eyelid of the respective eye at the respective time increment.

### Images.

Referring to Block 302, in some embodiments the method includes, for each respective time increment in the plurality of consecutive time increments, (i) obtaining a corresponding image of a respective eye (e.g., the first eye) comprising a corresponding plurality of pixels and one or more pixel values for each pixel in the corresponding plurality of pixels. Thus, in some implementations, the method includes obtaining a plurality of images for the respective subject, over the plurality of consecutive time increments.

In some embodiments, each corresponding image of the respective eye (e.g., the first eye) of the subject is obtained in electronic format.

In some embodiments, as described above (see, e.g., the section entitled “Eyelid traces”) the plurality of images for the respective subject comprises a respective series of sequential images for a respective subject, where the series of sequential images is obtained from the subject over the course of the plurality of consecutive time increments, and where the plurality of consecutive time increments has a non-zero duration, such as during a period of image capture (e.g., frames of a video and/or images taken during high-speed image capture).

In some embodiments, each respective image in a series of sequential images corresponds to a respective time increment during the plurality of consecutive time increments (e.g., frames of a video taken at a respective frame rate).

For example, in some embodiments, each respective image in the series of sequential images is captured at a rate of no more than 200 milliseconds (ms)/image, no more than 100 ms/image, no more than 90 ms/image, no more than 80 ms/image, no more than 70 ms/image, no more than 60 ms/image, no more than 50 ms/image, no more than 40 ms/image, no more than 30 ms/image, no 4 ms/image, no more than 3 ms/image, no more than 2 ms/image, no more than 1 ms/image, no more than 0.5 ms/image, no more than 0.1 ms/image. In some embodiments, each respective image in the series of sequential images is captured at a rate of from 0.1 to 50 ms/image, from 1 to 30 ms/image, from 0.5 to 100 ms/image, or from 1 to 10 ms/image. In some embodiments, each respective image in the series of sequential images is captured at a rate that falls within another range starting no lower than 0.1 ms/image and ending no higher than 200 ms/image.

In some embodiments, the plurality of images for the respective subject includes one or more images obtained from the subject while blinking. Thus, for example, the plurality of images for the respective subject can include one or more images obtained by recording the subject during a blink (e.g., a blink reflex), where the recording occurs at a given frame rate (e.g., 3 ms/image). In some instances, the recording is high-speed image capture.

In some embodiments, the blink is voluntary or involuntary. In some embodiments, a respective image in the plurality of images is obtained upon stimulation of at least one facial region of the subject using at least one stimulator so as to cause an involuntary blink response in the subject. In some embodiments, the at least one facial region is selected from the group consisting of the temple, the outer canthus, and the eye. In some embodiments, the stimulation of the at least one facial region is provided in proximity of the left eye, the right eye, or both eyes of the subject. Stimulation of at least one facial region of the subject includes any of the embodiments described in detail above (see, e.g., the section entitled “Stimuli”). In some embodiments, a respective image in the plurality of images is of the left eye, the right eye, or both eyes of the subject.

In some implementations, the stimulation of the at least one facial region is selected from the group consisting of: a puff of fluid (e.g., the fluid corresponding to a gas, a liquid, or a vapor); a mechanical contact (e.g., a pin prick); one or more flashes of light; an electrical current (e.g., less than approximately 1 milliamp); or a sound (e.g., between 70 and 80 decibels). In some implementations, the stimulation is a verbal or visual command. For example, in some embodiments, the stimulation is a verbal command (e.g., “blink”) administered to the subject. In some embodiments, the stimulation is a visual command (e.g., a screen that displays the word “blink”) that is presented to the subject.

In some embodiments, a respective image in the plurality of images is obtained from a subject that is afflicted with a neurological condition. In some embodiments, the neurological condition is a result of a traumatic event, a head impact, or a mild traumatic brain injury. Non-limiting examples of neurological conditions include Alzheimer's disease, Parkinson's disease, dementia, Huntington's disease, schizophrenia, migraines, stroke, and/or epilepsy. In some embodiments, a respective image in the plurality of images is obtained from a subject exhibiting a healthy and/or normal activity. For instance, in some embodiments, a respective image in the plurality of images is obtained from a subject prior to onset of a suspected neurological condition and/or obtained from a healthy control subject.

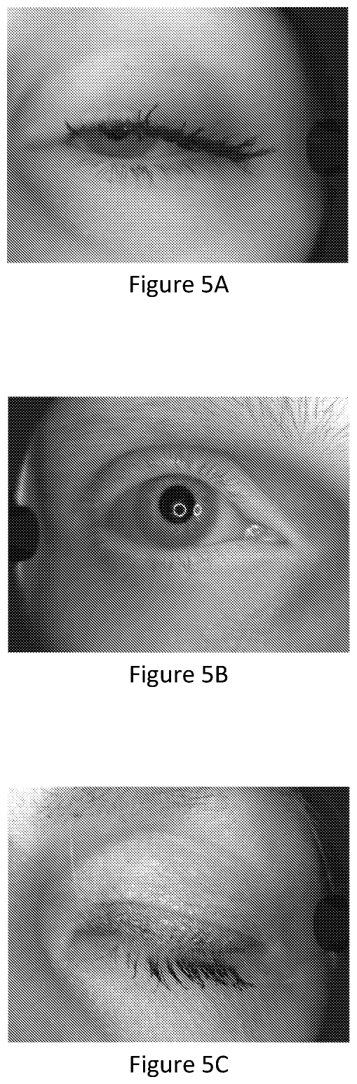

FIGS. 5A-C and 6 illustrate images of eyes of various subjects that can be used for obtaining eyelid traces, in accordance with some embodiments of the present disclosure. For instance, FIGS. 5A-C illustrate images captured at various positions (e.g., as can occur during a blink), including open (FIG. 5B), closed (FIG. 5C), and partial open (FIG. 5A).

Additional methods for obtaining images and stimulating blink reflexes are disclosed in, for example, U.S. patent application Ser. No. 14/787,564, filed May 1, 2014, International Patent Application No. PCT/US2018/032666, having an International Filing Date of May 15, 2018, U.S. Provisional Application No. 63/194,554, filed May 28, 2021, and/or U.S. Provisional Application No. 63/275,749, filed Nov. 4, 2021, each of which is hereby incorporated herein by reference in its entirety for all purposes. In addition, characteristics of blink responses are described in detail, for example, in Garner et al., 2018, “Blink reflex parameters in baseline, active, and head-impact Division I athletes,” Cogent Engineering, 5:1429110; doi: 10.1080/23311916.2018.1429110, which is hereby incorporated herein by reference in its entirety.

In some embodiments, the plurality of images includes multiple images for a single subject. In some embodiments, the plurality of images includes multiple batches of images (e.g., scans, videos, and/or series of images using high-speed image capture) for a single subject. In some embodiments, the multiple images and/or multiple batches of images for the single subject are obtained at a corresponding plurality of acquisition time points. In some embodiments, the plurality of images comprises, for a respective subject, a plurality of images obtained at 2 or more, 3 or more, 4 or more, 5 or more, 6 or more, 7 or more, 8 or more, 9 or more, or 10 or more different acquisition time points.

In some embodiments, an image is any form of two-dimensional RGB or grey-scaled pixelated image. For instance, in some embodiments, an image is a color image or a grey-scaled image. In some embodiments, an image comprises a size denoted by n x m, where n and m refer to an edge length, in pixels. Referring to Block 304, in some embodiments, an edge length, in pixels, of the corresponding image consists of between 164 pixels and 1024 pixels. In some embodiments, an edge length of an image in the plurality of images is at least 50, at least 100, at least 150, at least 200, at least 250, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900, or at least 2000 pixels. In some embodiments, an edge length of an image in the plurality of images is no more than 2500, no more than 2000, no more than 1600, no more than 1000, no more than 800, no more than 600, no more than 400, no more than 300, or no more than 200 pixels. In some embodiments, an edge length of an image in the plurality of images is from 50 to 800, from 200 to 600, from 300 to 900, from 500 to 1800, or from 50 to 2500 pixels. In some embodiments, an edge length of an image in the plurality of images falls within another range starting no lower than 50 pixels and ending no higher than 2500 pixels. In some embodiments, n and m are the same value. In some embodiments, n and m are different values.

Generally, the plurality of pixels corresponding to a respective image of an eye refers to the image resolution (e.g., the number of pixels in an image). In some instances, the image resolution can be determined by the dimensions of the image (e.g., n x m). Thus, in some embodiments, the plurality of pixels corresponding to a respective image of an eye in the plurality of images comprises at least 2000, at least 5000, at least 10,000, at least 20,000, at least 30,000, at least 40,000, at least 50,000, at least 100,000, at least 250,000, at least 500,000, at least 1 million, at least 1.5 million, at least 2 million, at least 3 million, or at least 5 million pixels. In some embodiments, the plurality of pixels corresponding to a respective image of an eye in the plurality of images comprises no more than 10 million, no more than 5 million, no more than 1 million, no more than 500,000, no more than 100,000, no more than 50,000, or no more than 10,000 pixels. In some embodiments, the plurality of pixels corresponding to a respective image of an eye in the plurality of images comprises from 5000 to 50,000, from 10,000 to 500,000, from 50,000 to 1 million, or from 1 million to 3 million pixels. In some embodiments, the plurality of pixels falls within another range starting no lower than 2000 pixels and ending no higher than 10 million pixels. In some embodiments, the plurality of pixels comprises any number of pixels determined using the values for edge length, in pixels, of an image disclosed herein.

In some embodiments, one or more images in the plurality of images are downscaled or upscaled (e.g., where the resolution is decreased or increased) in order to fit a target size. In some embodiments, each respective image in the plurality of images is downscaled or upscaled in order to match a target size.

In some embodiments, the one or more pixel values for each pixel in the corresponding plurality of pixels are RGB values. For instance, in some embodiments, the image is a color image, and the one or more pixel values for each pixel in the corresponding plurality of pixels are RGB values for the color image. In some embodiments, the one or more pixel values for each pixel in the corresponding plurality of pixels are grey-scaled values (e.g., pixel intensity). In some embodiments, the one or more pixel values for each pixel in the corresponding plurality of pixels are stored in a corresponding one or more channels (e.g., in a tensor). For instance, three channels can be used to store RGB values for each pixel in the plurality of pixels, whereas one channel can be used to store a grey-scaled pixel value for each pixel in the plurality of pixels. In some embodiments, each pixel value in the one or more pixel values is from 0 to 255.

In some embodiments, a respective image in the plurality of images comprises an annotation associated with the respective image, where the annotation includes any category, characteristic, or identifying feature of the image, including but not limited to a tag, filename, subject identity, scan number, batch number, timestamp, frame number, demographic data (e.g., gender, age, clinical condition, neurological condition, cohort, etc.) of the subject, image metadata (e.g., image size, image resolution, type of preprocessing used, etc.), eyelid position (open, closed, partial open, etc.), and/or image input group (e.g., training, validation, test, etc.).

In some embodiments, a corresponding image of a respective eye comprises a visual modification of the eye of the subject. In some embodiments, the visual modification of the eye of the subject is a modification of the image of the eye and/or a modification of the physical eye of the subject. In some embodiments, the visual modification of the eye of the subject comprises a difference in eyelid position (e.g., closed, open, partial open, etc.), image acquisition quality (e.g., lighting, resolution, etc.), framing (e.g., angle, cropping, etc.), and/or texture or surface modifications (e.g., application of cosmetics, color, contact lenses, etc.). For example, in some embodiments, the visual modification of the eye of the subject comprises an application of cosmetics (e.g., eyeliner, mascara, etc.) to the eye.

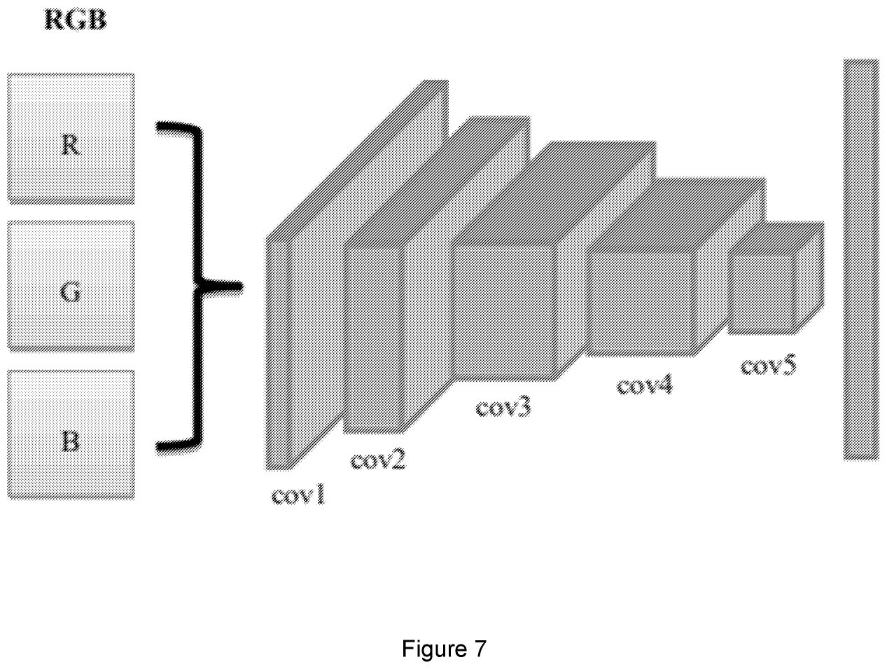

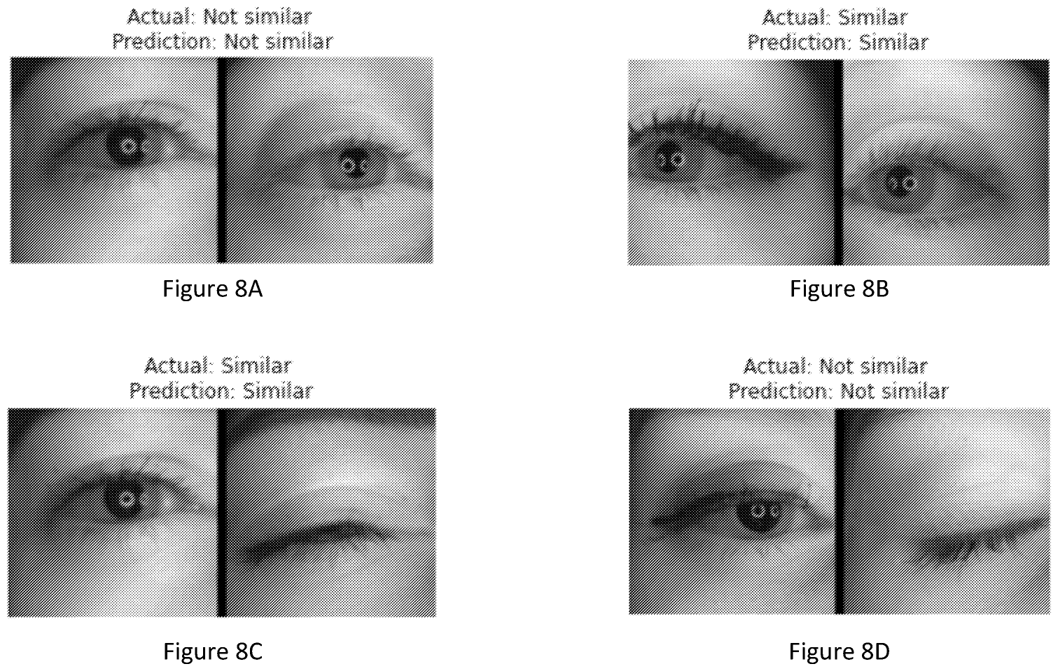

Accordingly, in some embodiments, the method further includes determining whether a first respective image is of the same class (e.g., obtained from the same subject) as a second respective image in a plurality of images, even when the first image and the second image comprise visual modifications that impart dissimilarities between them (e.g., an image of an eye from a subject with and without cosmetics). See, for example, a comparison between two eyes from the same subject in FIG. 8B.

In some embodiments, the method further includes generating a report indicating whether a first corresponding image and a second corresponding image are of the same corresponding class (e.g., obtained from the same subject). For instance, in some embodiments, the generating a report comprises, when the first and second image are determined to be of the same corresponding class, the first and second image are deemed to be similar, and when the first and second image are determined to be of different corresponding classes, the first and second image are deemed to be not similar. Example outputs are further described in Example 2, below, with reference to FIGS. 8A-D.

In some embodiments, the method further comprises normalizing the one or more pixel values for each pixel in the corresponding plurality of pixels of each corresponding image. In some embodiments, the normalizing normalizes a mean value of the pixel values of the corresponding plurality of pixels of each corresponding image to a predetermined value. In some embodiments, the normalizing normalizes a standard deviation of the pixel values of the corresponding plurality of pixels of each corresponding image to a predetermined standard deviation. Generally, normalization of pixels in a respective image improves performance by reducing outliers in the image data.

In some embodiments, the one or more pixel values for each corresponding image are normalized across the plurality of images for the respective subject. In some embodiments, the one or more pixel values for each corresponding image in a batch of images are normalized across the plurality of images in the batch. In some embodiments, the one or more pixel values for each corresponding image are normalized across a plurality of subjects. In some embodiments, one or more pixel values for a corresponding image are not normalized.

In some embodiments, the normalizing normalizes a mean value of the pixel values of the corresponding plurality of pixels of each corresponding image of a plurality of images (e.g., corresponding to a subject, a batch, and/or a plurality of subjects) to a predetermined value, where the predetermined value is a measure of central tendency (e.g., a mean, median, mode, etc.) determined for the respective plurality of images (e.g., corresponding to the subject, the batch, and/or the plurality of subjects). In some embodiments, the normalizing normalizes a standard deviation of the pixel values of the corresponding plurality of pixels of each corresponding image of the plurality of images (e.g., corresponding to a subject, a batch, and/or a plurality of subjects) to a predetermined value, where the predetermined value is a measure of dispersion (e.g., a standard deviation, standard error, confidence interval, etc.) determined for the respective plurality of images (e.g., corresponding to the subject, the batch, and/or the plurality of subjects). In some embodiments, the predetermined value (e.g., for the mean and/or the standard deviation of the pixel values) is a value that is defined by a user or practitioner (e.g., an explicit statistic). In some embodiments, the predetermined value (e.g., for the mean and/or the standard deviation of the pixel values) is a predetermined statistic determined by a transfer-learned neural network (e.g., a pre-trained ResNet).

In some embodiments, a corresponding image of a respective eye is further preprocessed. In some embodiments, each corresponding image of the respective eye is further preprocessed. In some embodiments, the preprocessing comprises one or more of resizing, tensor conversion, normalization, and/or reordering channels (e.g., permutation).

In some embodiments, the method comprises combining two or more images (e.g., frames) in the plurality of images for the respective subject. In some embodiments, the method comprises adding an image to the plurality of images for the respective subject. In some embodiments, the method comprises deleting one or more images from the plurality of images for the respective subject. In some such embodiments, the deleting comprises detecting one or more images that do not include an eyelid and removing the one or more images from the plurality of images. In some embodiments, the deleting comprises detecting one or more images that do not include an eye and removing the one or more images from the plurality of images. In some embodiments, the method comprises, when a respective one or more images in the plurality of images are combined, added, and/or deleted, recalculating a frame rate for the plurality of images.

Any suitable method and/or embodiment disclosed herein for obtaining images, such as those described in further detail above in the section entitled “Eyelid traces,” is contemplated for use in the present disclosure, as well as any substitutions, modifications, additions, deletions, and/or combinations thereof, as will be apparent to one skilled in the art. In some embodiments, a respective image used for obtaining traces and/or determining eye closure status includes an image used as input for training a neural network, as disclosed herein. Moreover, in some embodiments, a respective image used as input for training a neural network includes any of the embodiments of a respective image used for obtaining traces and/or determining eye closure status disclosed herein.

Other suitable embodiments for images contemplated for use in the present disclosure are described in further detail in, for example, U.S. patent application Ser. No. 14/787,564, filed May 1, 2014, International Patent Application No. PCT/US2018/032666, having an International Filing Date of May 15, 2018, U.S. Provisional Application No. 63/194,554, filed May 28, 2021, and/or U.S. Provisional Application No. 63/275,749, filed Nov. 4, 2021, each of which is hereby incorporated herein by reference in its entirety for all purposes.

### Determining Eyelid Locations.

Referring to Block 306, in some embodiments the method further includes, for each respective time increment in the plurality of consecutive time increments, (ii) inputting the corresponding image into a trained neural network comprising a plurality of parameters (e.g., 10,000 or more parameters), thereby obtaining the respective location of a respective eyelid (e.g., the lower eyelid and/or the upper eyelid) of the respective eye (e.g., the first eye) at the respective time increment.

As described above, in some embodiments, the respective location of a respective eyelid is a respective calculated set of coordinates that localizes the respective eyelid in the corresponding image. See, for example, the section entitled, “Eyelid traces,” above.

In some embodiments, a set of coordinates corresponding to an eyelid (e.g., an upper eyelid and/or a lower eyelid) indicates the center of the eyelid. In some embodiments, the set of coordinates indicates an edge of an eyelid (e.g., a bottom edge of an upper eyelid and/or an upper edge of a bottom eyelid). In some embodiments, the set of coordinates indicates the center of the edge of an eyelid. In some embodiments, a set of coordinates corresponding to an eyelid is measured from the top left of the eyelid and/or the top left of the image containing the eyelid (e.g., where the top left of the eyelid and/or the image has the coordinates (0,0)).

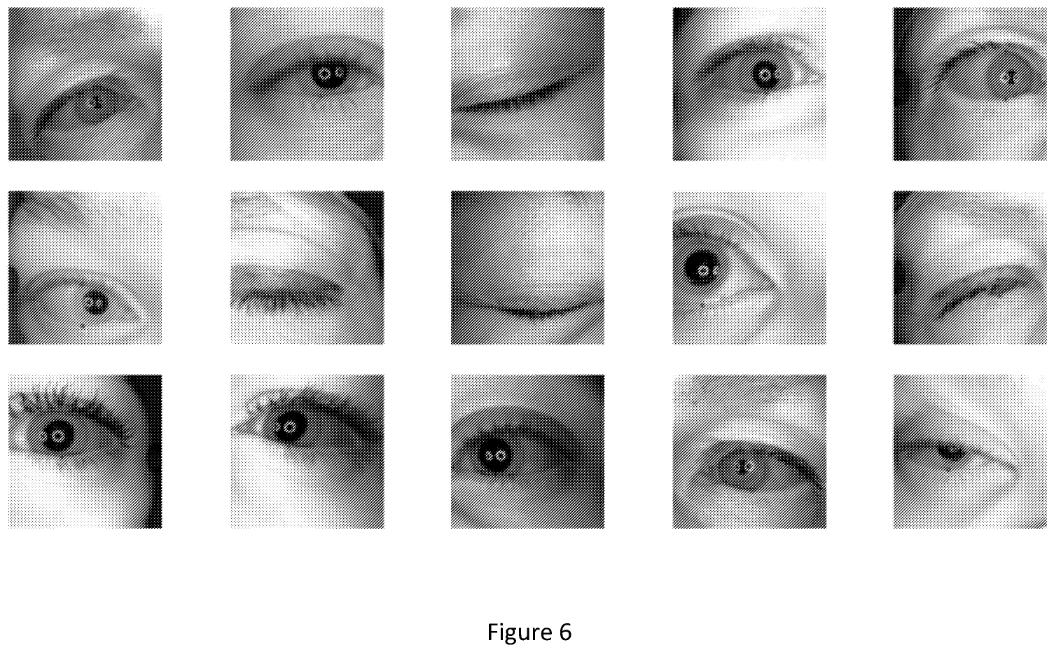

Thus, for an upper eyelid and a lower eyelid of a corresponding image for a respective subject, a corresponding first calculated set of coordinates and a corresponding second calculated set of coordinates provides two sets of coordinates (e.g., (x1, y1) and (x2, y2)) that localize the upper eyelid and the lower eyelid, respectively. As an example, FIG. 6 illustrates various “keypoints” (e.g., first and second calculated sets of coordinates), where highlighted points on each image indicate annotated centers of the upper and lower eyelids in each image.

In some embodiments, the calculated set of coordinates for each respective corresponding image in the plurality of images for the respective subject is provided as a list of values indicating the x and y coordinates for corresponding images, which can be read by a computing system and/or a non-transitory computer readable storage medium. In some embodiments, the corresponding images are further provided as image files, file names, and/or file paths that can be read by a computing system and/or a non-transitory computer readable storage medium.

In some embodiments, the obtaining the respective location of a respective eyelid in a corresponding image is performed for a lower eyelid, an upper eyelid, or both eyelids of a respective eye.

In some embodiments, the obtaining the respective location of one or more respective eyelids in a corresponding image of a respective eye is performed for a left eye, a right eye, or both eyes of a respective subject.

In some embodiments, the obtaining the respective location of one or more respective eyelids in one or more corresponding images for a left eye and/or a right eye of a respective subject is performed for each respective subject in a plurality of subjects.

### Model Architecture.

Referring to Block 308, the neural network comprises a plurality of convolutional layers, where each convolutional layer in the plurality of convolutional layers comprises one or more filters (e.g., kernels), a respective size (e.g., n x n), and a respective stride, and one or more pooling layers, where each pooling layer in the one or more pooling layers comprises a respective size and a respective stride.

In some embodiments, each corresponding image that is inputted to the neural network is restricted by the input shape of a first layer in the neural network. In some such embodiments, the size of a respective layer (e.g., n x n) defines the input shape of the layer. In some embodiments, the corresponding image is reshaped prior to input to the trained neural network (e.g., converted from a three-dimensional matrix to a one-dimensional vector).

For instance, referring to Block 310, in some embodiments, the neural network comprises an initial convolutional neural network layer that receives a grey-scaled pixel value for each pixel in the corresponding plurality of pixels as input into the neural network, where the initial convolutional neural network layer includes a first activation function, and where the initial convolutional neural network layer convolves the corresponding plurality of pixels into more than 10 separate parameters for each pixel in the corresponding plurality of pixels.

In some embodiments, the neural network comprises an initial convolutional neural network layer that receives one or more color (e.g., RGB) pixel values for each pixel in the corresponding plurality of pixels as input into the neural network, where the initial convolutional neural network layer includes a first activation function, and where the initial convolutional neural network layer convolves the corresponding plurality of pixels into more than 10 separate parameters for each pixel in the corresponding plurality of pixels.

In some embodiments, the more than 10 separate parameters for each pixel in the plurality of pixels comprises a corresponding more than 10 separate channels for each pixel in the plurality of pixels. For instance, in some embodiments, the convolution comprises “windowing” a kernel of a specified size (e.g., 2×2, 3×3, etc.) across the plurality of pixels. As the kernel moves along (e.g., in accordance with a specified stride), each window is convolved according to a specified function (e.g., an activation function, average pooling, max pooling, etc.). In some embodiments, the convolution further convolves the corresponding plurality of pixels into a plurality of output channels. In some embodiments, the number of output channels for each respective pixel in the plurality of pixels increases or decreases in accordance with a specified output size (e.g., from 1 input channel to more than 10 output channels after the first pooling layer).

Accordingly, referring to Block 312, in some embodiments, the neural network further comprises a pooling layer that pools the 10 separate parameters for each pixel in the plurality of pixels outputted by the initial convolutional neural network layer. In some embodiments, a respective pooling layer (e.g., downsampling layer) is used to reduce the number of pixels and/or parameters associated with an input image (e.g., to reduce the computational complexity). For instance, an average pooling function windows across the plurality of pixels, in accordance with the kernel size and the stride, and averages, for each window, the one or more pixel values encompassed by the kernel. Alternatively, a max pooling function windows across the plurality of pixels, in accordance with the kernel size and the stride, and pools the one or more pixel values windowed by the kernel by selecting the maximum value within the kernel.

In some embodiments, the neural network further comprises a plurality of intermediate blocks including a first intermediate block and a final intermediate block, where the first intermediate block takes as input the output of the pooling layer, each intermediate block in the plurality of intermediate blocks other than the first intermediate block and the final intermediate block takes, as input, an output of another intermediate block in the plurality of intermediate blocks and has an output that serves as input to another intermediate block in the plurality of intermediate blocks, and each intermediate block comprises a respective first convolutional layer comprising a set of parameters (e.g., more than 1000 parameters), where the respective convolutional layer has a corresponding activation function.

In some embodiments, each intermediate block in the plurality of intermediate blocks comprises a corresponding second convolutional layer that takes, as input, an output of the respective first convolutional layer.

In some such embodiments, each intermediate block in the plurality of intermediate blocks comprises a merge layer that merges (i) an output of the respective second convolutional layer and (ii) an output of a preceding intermediate block in the plurality of intermediate blocks. In some embodiments, each intermediate block in the plurality of intermediate blocks has a corresponding input size and a corresponding output size, and, when the corresponding input size of a respective intermediate block differs from the corresponding output size, the respective intermediate block further comprises a corresponding third convolutional layer that receives, as input, the (ii) output of the preceding intermediate block, where the corresponding third convolutional layer convolves the (ii) output of the preceding intermediate block prior to the merging (i) and (ii) by the merge layer. Thus, in some such embodiments, when the shape of the data changes between intermediate blocks, the method comprises using a third convolutional layer that preprocesses the previous block's output such that it can be merged with the current block's output.

In some embodiments, the final intermediate block takes, as input, an output of another intermediate block in the plurality of intermediate blocks and produces, as output, a flattened data structure comprising a predetermined plurality of values. In some embodiments, the flattened data structure is a one-dimensional vector comprising the predetermined plurality of values. In some embodiments, the predetermined plurality of values comprises from 10 to 1000, from 200 to 800, from 100 to 2000, from 500 to 5000, or from 1000 to 10,000 values.

In some embodiments, the final intermediate block further comprises, prior to the flattening of the data, decreasing the number of channels in the plurality of channels in the input provided as input to the final intermediate block. In some embodiments, the final intermediate block further comprises, prior to the flattening of the data, decreasing the number of pixels in the plurality of pixels provided as input to the final intermediate block.

In some embodiments, each value in the predetermined plurality of values is a value between −1 and 1. In some embodiments, each value in the predetermined plurality of values is a value between 0 and 1.

In some embodiments, the neural network comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, or at least 100 intermediate blocks. In some embodiments, the neural network comprises no more than 100, no more than 90, no more than 80, no more than 70, no more than 60, no more than 50, no more than 40, no more than 30, no more than 20, no more than 10, no more than 9, no more than 8, no more than 7, no more than 6, or no more than 5 intermediate blocks. In some embodiments, the neural network comprises from 1 to 5, from 1 to 10, from 1 to 20, from 10 to 50, from 2 to 80, from 5 to 100, from 10 to 100, from 50 to 100, or from 3 to 30 intermediate blocks. In some embodiments, the neural network comprises a plurality of hidden layers that falls within another range starting no lower than 1 intermediate block and ending no higher than 100 intermediate blocks.

In some embodiments, the neural network further comprises a regressor block including a first dropout layer, a first linear layer, and a corresponding activation function, where the regressor block takes, as input, the flattened data structure comprising the predetermined plurality of values. In some embodiments, the regressor block does not include a dropout layer.

In some embodiments, the first dropout layer removes a first subset of values from the plurality of values in the flattened data structure, based on a first dropout rate. In some embodiments, the first linear layer applies a first linear transformation to the plurality of values in the flattened data structure.

In some embodiments, the regressor block further includes a second dropout layer, where the second dropout layer removes a second subset of values from the plurality of values in the flattened data structure, based on a second dropout rate. In some embodiments, the regressor block further includes a second linear layer, where the second linear layer applies a second linear transformation to the plurality of values in the flattened data structure.

In some embodiments, a respective linear layer (e.g., a first linear layer and/or a second linear layer) applies a matrix multiplication to the plurality of values in the flattened data structure.

In some embodiments, the regressor block produces, as output, a corresponding first calculated set of coordinates that localize a first eyelid (e.g., a lower eyelid) in the corresponding image. In some embodiments, the regressor block produces, as output, a corresponding second calculated set of coordinates that localize a second eyelid (e.g., an upper eyelid) in the corresponding image.

In some embodiments, the neural network comprises a plurality of parameters (e.g., weights and/or hyperparameters). In some embodiments, each respective layer and/or block in the neural network comprises a respective corresponding plurality of parameters. In some such embodiments, the respective corresponding plurality of parameters for a respective layer and/or block is a subset of the plurality of parameters associated with the neural network.

In some embodiments, the plurality of parameters for the trained neural network comprises at least 10, at least 50, at least 100, at least 500, at least 1000, at least 2000, at least 5000, at least 10,000, at least 20,000, at least 50,000, at least 100,000, at least 200,000, at least 500,000, at least 1 million, at least 2 million, at least 3 million, at least 4 million or at least 5 million parameters. In some embodiments, the plurality of parameters for the trained neural network comprises no more than 8 million, no more than 5 million, no more than 4 million, no more than 1 million, no more than 500,000, no more than 100,000, no more than 50,000, no more than 10,000, no more than 5000, no more than 1000, or no more than 500 parameters. In some embodiments, the plurality of parameters for the trained neural network comprises from 10 to 5000, from 500 to 10,000, from 10,000 to 500,000, from 20,000 to 1 million, or from 1 million to 5 million parameters. In some embodiments, the plurality of parameters for the trained neural network falls within another range starting no lower than 10 parameters and ending no higher than 8 million parameters.

In some embodiments, the plurality of parameters for a first layer of the trained neural network comprises at least 10, at least 50, at least 100, at least 500, at least 1000, at least 2000, at least 3000, at least 4000, at least 5000, at least 7500, at least 10,000, at least 20,000, at least 30,000, at least 40,000, at least 50,000, at least 100,000, at least 200,000, or at least 500,000 parameters. In some embodiments, the plurality of parameters for a first layer of the trained neural network comprises no more than 1 million, no more than 500,000, no more than 100,000, no more than 50,000, no more than 10,000, no more than 5000, no more than 1000, or no more than 500 parameters. In some embodiments, the plurality of parameters for a first layer of the trained neural network comprises from 10 to 5000, from 500 to 10,000, from 1000 to 5000, from 1000 to 100,000, or from 10,000 to 500,000 parameters. In some embodiments, the plurality of parameters for a first layer of the trained neural network falls within another range starting no lower than 10 parameters and ending no higher than 1 million parameters.

In some embodiments, the plurality of parameters for a respective layer in a plurality of layers in the trained neural network comprises at least 100, at least 500, at least 1000, at least 2000, at least 5000, at least 10,000, at least 20,000, at least 50,000, at least 100,000, at least 200,000, at least 500,000, at least 1 million, at least 2 million, or at least 3 million parameters. In some embodiments, the plurality of parameters for a respective layer in a plurality of layers in the trained neural network comprises no more than 5 million, no more than 4 million, no more than 1 million, no more than 500,000, no more than 100,000, no more than 50,000, no more than 10,000, no more than 5000, or no more than 1000 parameters. In some embodiments, the plurality of parameters for a respective layer in a plurality of layers in the trained neural network comprises from 100 to 1000, from 1000 to 10,000, from 2000 and 200,000, from 8000 and 1 million, or from 30,000 and 3 million parameters. In some embodiments, the plurality of parameters for a respective layer in a plurality of layers in the trained neural network falls within another range starting no lower than 100 parameters and ending no higher than 5 million parameters.

In some embodiments, the neural network comprises a plurality of hidden layers. Generally, as described above, hidden layers are located between input and output layers (e.g., to capture additional complexity). In some embodiments, where there is a plurality of hidden layers, each hidden layer may have the same or a different respective number of hidden neurons.

In some embodiments, each hidden neuron (e.g., in a respective hidden layer in a neural network) is associated with an activation function that performs a function on the input data (e.g., a linear or non-linear function). Generally, the purpose of the activation function is to introduce nonlinearity into the data such that the neural network is trained on representations of the original data and can subsequently “fit” or generate additional representations of new (e.g., previously unseen) data. Selection of activation functions (e.g., a first and/or a second activation function) is dependent on the use case of the neural network, as certain activation functions can lead to saturation at the extreme ends of a dataset (e.g., tanh and/or sigmoid functions). For instance, in some embodiments, an activation function (e.g., a first and/or a second activation function) is selected from any of the activation functions disclosed herein and described in greater detail below.

In some embodiments, each hidden neuron is further associated with a parameter (e.g., a weight and/or a bias value) that contributes to the output of the neural network, which is determined based on the associated activation function. In some embodiments, prior to training, the hidden neuron is initialized with arbitrary parameters (e.g., randomized weights). In some alternative embodiments, prior to training, the hidden neuron is initialized with a predetermined set of parameters.

In some embodiments, the plurality of hidden neurons in a neural network (e.g., across one or more hidden layers) is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, at least 100, at least 200, at least 300, at least 400, or at least 500 neurons. In some embodiments, the plurality of hidden neurons is at least 100, at least 500, at least 800, at least 1000, at least 2000, at least 3000, at least 4000, at least 5000, at least 6000, at least 7000, at least 8000, at least 9000, at least 10,000, at least 15,000, at least 20,000, or at least 30,000 neurons. In some embodiments, the plurality of hidden neurons is no more than 30,000, no more than 20,000, no more than 15,000, no more than 10,000, no more than 9000, no more than 8000, no more than 7000, no more than 6000, no more than 5000, no more than 4000, no more than 3000, no more than 2000, no more than 1000, no more than 900, no more than 800, no more than 700, no more than 600, no more than 500, no more than 400, no more than 300, no more than 200, no more than 100, or no more than 50 neurons. In some embodiments, the plurality of hidden neurons is from 2 to 20, from 2 to 200, from 2 to 1000, from 10 to 50, from 10 to 200, from 20 to 500, from 100 to 800, from 50 to 1000, from 500 to 2000, from 1000 to 5000, from 5000 to 10,000, from 10,000 to 15,000, from 15,000 to 20,000, or from 20,000 to 30,000 neurons. In some embodiments, the plurality of hidden neurons falls within another range starting no lower than 2 neurons and ending no higher than 30,000 neurons.

In some embodiments, the neural network comprises from 1 to 50 hidden layers. In some embodiments, the neural network comprises from 1 to 20 hidden layers. In some embodiments, the neural network comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 30, at least 40, at least 50, at least 60, at least 70, at least 80, at least 90, or at least 100 hidden layers. In some embodiments, the neural network comprises no more than 100, no more than 90, no more than 80, no more than 70, no more than 60, no more than 50, no more than 40, no more than 30, no more than 20, no more than 10, no more than 9, no more than 8, no more than 7, no more than 6, or no more than 5 hidden layers. In some embodiments, the neural network comprises from 1 to 5, from 1 to 10, from 1 to 20, from 10 to 50, from 2 to 80, from 5 to 100, from 10 to 100, from 50 to 100, or from 3 to 30 hidden layers. In some embodiments, the neural network comprises a plurality of hidden layers that falls within another range starting no lower than 1 layer and ending no higher than 100 layers.

In some embodiments, the neural network comprises a shallow neural network. A shallow neural network refers to a neural network with a small number of hidden layers. In some embodiments, such neural network architectures improve the efficiency of neural network training and performance and conserve computational power due to the reduced number of layers involved. In some embodiments, the neural network comprises only one hidden layer.

As described above, in some embodiments, one or more layers and/or blocks in the trained neural network is associated with one or more activation functions. Referring to Block 314, in some embodiments, a respective activation function is tanh, sigmoid, softmax, Gaussian, Boltzmann-weighted averaging, absolute value, linear, rectified linear unit (ReLU), bounded rectified linear, soft rectified linear, parameterized rectified linear, average, max, min, sign, square, square root, multiquadric, inverse quadratic, inverse multiquadric, polyharmonic spline, swish, mish, Gaussian error linear unit (GeLU), scaled exponential linear unit (SELU), or thin plate spline.

As described above, in some embodiments, one or more layers and/or blocks in the trained neural network is associated with one or more average pooling and/or max pooling functions.

In some embodiments, the trained neural network is further characterized by one or more hyperparameters. In some embodiments, the hyperparameter values are tuned (e.g., adjusted) when training the neural network. In some embodiments, the hyperparameter values are determined based on the specific elements of one or more input images (e.g., number of images, number of pixels, number of channels, etc.). In some embodiments, the hyperparameter values are determined using experimental optimization. In some embodiments, the hyperparameter values are determined using a hyperparameter sweep. In some embodiments, the hyperparameter values are assigned based on prior template or default values.

In some embodiments, a respective hyperparameter of the one or more hyperparameters comprises a stride (e.g., a number of pixels by which a kernel moves across an image). In some embodiments, the stride is at least 1, at least 2, at least 3, at least 4, at least 5, at least 6, at least 7, at least 8, at least 9, or at least 10. In some embodiments, the stride is no more than 50, no more than 30, no more than 20, no more than 10, no more than 9, no more than 8, no more than 7, no more than 6, no more than 5, no more than 4, no more than 3, no more than 2, or no more than 1. In some embodiments, the stride is from 1 to 10, from 2 to 30, or from 5 to 50. In some embodiments, the stride falls within another range starting no lower than 1 and ending no higher than 50.

In some embodiments, a respective layer and/or block has the same or different values for a respective hyperparameter (e.g., stride). For example, referring to Block 316, in some embodiments, the initial convolutional neural network layer has a stride of two or more.

In some embodiments, a respective hyperparameter of the one or more hyperparameters comprises a kernel size. Generally, kernels (e.g., convolutional filters) comprise a corresponding height and width. In typical embodiments, a respective kernel (e.g., filter) is smaller than the input image to the corresponding convolutional layer which the respective kernel is used to convolve. In some embodiments, the value for a kernel size indicates the edge length of the kernel (e.g., 1×1, 2×2, etc.). In some embodiments, a respective size for a kernel is a matrix (n×n) of pixels. In some embodiments, n is at least 1, at least 2, at least 3, at least 4, at least 5, at least 6, at least 7, at least 8, at least 9, at least 10, at least 20, at least 30, at least 40, or at least 50. In some embodiments, n is at most 100, at most 50, at most 10, at most 9, at most 8, at most 7, at most 6, at most 5, at most 4, at most 3, at most 2, or at most 1. In some embodiments, n is from 1 to 10, from 2 to 30, from 5 to 50, or from 10 to 100. In some embodiments, n falls within another range starting no lower than 1 and ending no higher than 100.

In some embodiments, a respective hyperparameter of the one or more hyperparameters comprises padding (e.g., the width in pixels of a border that is applied around the edges of an image while it is being convolved, so that data at the edges of the image are not lost during the convolution). In some embodiments, the padding is at least 1, at least 2, at least 3, at least 4, at least 5, at least 6, at least 7, at least 8, at least 9, at least 10, at least 20, at least 30, at least 40, or at least 50 pixels. In some embodiments, the padding is at most 100, at most 50, at most 10, at most 9, at most 8, at most 7, at most 6, at most 5, at most 4, at most 3, at most 2, or at most 1 pixel. In some embodiments, the padding is from 1 to 10, from 2 to 30, from 5 to 50, or from 10 to 100 pixels. In some embodiments, the padding falls within another range starting no lower than 1 pixel and ending no higher than 100 pixels.

In some embodiments, a respective hyperparameter of the one or more hyperparameters comprises an input size. In some embodiments, a respective hyperparameter of the one or more hyperparameters comprises an output size. In some embodiments, the input size is a first number of dimensions and the output size is a second number of dimensions. For instance, in some embodiments, the input size indicates a first number of channels that is received as input to a respective layer and/or block in the trained neural network. In some embodiments, the output size indicates a second number of channels that is outputted by the respective layer and/or block in the trained neural network. In some embodiments, the input size and/or the output size is referred to as the data shape.

In some embodiments, an output of a respective layer comprises a second number of dimensions that is different (e.g., less or more) from the first number of dimensions (e.g., the data shape changes between layers and/or blocks). In some embodiments, the input size and/or the output size (e.g., a number of channels) can be specified for any one or more layers and/or blocks in the trained neural network.

In some embodiments, the number of channels (e.g., input size and/or output size) of a respective layer and/or block in the trained neural network comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 150, at least 200, at least 250, at least 300, at least 400, or at least 500 channels. In some embodiments, the number of channels (e.g., input size and/or output size) of a respective layer and/or block in the trained neural network comprises at most 1000, at most 500, at most 100, at most 90, at most 80, at most 70, at most 60, or at most 50 channels. In some embodiments, the number of channels (e.g., input size and/or output size) of a respective layer and/or block in the trained neural network is from 1 to 40, from 10 to 100, from 50 to 500, or from 100 to 1000 channels. In some embodiments, the number of channels (e.g., input size and/or output size) of a respective layer and/or block in the trained neural network falls within another range starting no lower than 1 and ending no higher than 1000 channels.

Thus, in an example embodiment, a respective layer in the plurality of layers in the neural network is associated with a plurality of hyperparameters (e.g., input channels, output channels, kernel, stride, and/or padding). The plurality of hyperparameters defines the how the input to the respective layer is processed to produce the output of the respective layer. In some instances, the number of pixels in the plurality of pixels for the image (and/or in a tensor representation of the image) will decrease after being processed by a respective layer in the plurality of layers. Alternatively, or additionally, in some instances, the number of channels will increase after being processed by a respective layer in the plurality of layers. Thus, in some such instances, while convolution can decrease the number of pixels in a corresponding image, it can also concurrently increase the number of channels in the tensor representation of the image.

FIG. 7 illustrates the example situation in which an image progresses through a convolutional neural network. Successive rounds of convolution (e.g., cov1, cov2, cov3, cov4, and cov5) result in a corresponding successive reduction of the number of “pixels” outputted by a preceding layer and inputted to a subsequent layer. However, variation in the number of channels can be observed between convolutional layers, as illustrated by the varying depth of layers 1-5 as the tensor passes through each respective layer. While FIG. 7 illustrates one or more pixels comprising RGB pixel values as an initial input to the convolutional neural network, in practice a plurality of pixels comprising grey-scaled pixel values can be presented as an initial input to the convolutional neural network.

In some embodiments, any respective layer and/or block has the same or different values for a respective hyperparameter as any other respective layer and/or block. The arrangement of convolutional filters, residual blocks, pooling layers, parameters, and/or hyperparameters, and the number of such filters, blocks, layers, parameters, and/or hyperparameters in the neural networks disclosed herein are exemplary and other embodiments of the present disclosure may include other arrangements of such layers as will be apparent to one skilled in the art. Additionally, in some embodiments, the neural network can be constructed with any of the layers and/or blocks in any order and/or having any variations in parameters (e.g., type, number, and/or values thereof) that will be apparent to one skilled in the art.

In some embodiments, the trained neural network is any neural network disclosed herein (see, Definitions: Classifiers). In some embodiments, the trained neural network further comprises any algorithm and/or model disclosed herein (see, Definitions: Classifiers). For instance, in some embodiments, the trained neural network comprises a multilayer neural network, a deep convolutional neural network, a visual geometry convolutional neural network, a residual neural network, a residual convolutional neural network, and/or a combination thereof.

Referring to Block 318, in some example embodiments, the neural network is LeNet, AlexNet, VGGNet 16, GoogLeNet, ResNet, SE-ResNeXt, MobileNet, or EfficientNet. Each of these specific neural networks is designed for use on image data. In some embodiments, any neural network designed to analyze image data can be used as the trained neural network. However, in some embodiments the multi-categorical classification layers, which may have more than 1000 classifications, may be replaced with a coordinate prediction layer (e.g., a regressor block). In some embodiments, the multi-categorical classification layers may be replaced with a plurality of coordinate prediction layers.

LeNet can be one type of neural network, and variations have been developed for use with image data. In some embodiments, LeNet can comprise at least one convolutional layer, at least one pooling layer, and at least one fully-connected layer. In some embodiments, LeNet comprises a first convolutional layer, a first pooling layer (e.g., a sub-sampling layer), a second convolutional layer, a second pooling layer, a first fully-connected layer, a second fully-connected layer, and a classification (e.g., output) layer.

AlexNet can be a GPU-enabled neural network for image recognition. In some embodiments, AlexNet can comprise at least eight convolutional, pooling, and fully-connected layers. In some embodiments, AlexNet includes five convolutional layers and three fully-connected layers.

VGGNet can be used to reduce the number of parameters. In some embodiments, VGGNet comprises 13 convolutional layers, 5 max-pooling layers, 2 fully-connected layers, and a classifying layer. In some embodiments, VGGNet alternates at least two convolutional layers with a pooling layer.

GoogLeNet can use dimensionality reduction initially to reduce computation usage, which also helps to prevent overfitting. GoogLeNet comprises fewer than 30 layers.

ResNet can be a deep network that includes a high number of layers (e.g., at least 25 layers, at least 50 layers, at least 100 layers, at least 150 layers, at least 200 layers, at least 300 layers, at least 400 layers, at least 500 layers, at least 750 layers, at least 1000 layers, at least 200 layers, at least 3000 layers, at least 4000 layers or at least 5000 layers) and provide for skipping of one or more layers. The possibility of skipping over some of the layers can keep the performance of the neural network from degrading too much. In some embodiments, ResNet can comprise at least 1000 layers.

Alternatively or additionally, in some embodiments, the neural network includes a first portion and a second portion. In some embodiments, the neural network includes at least two portions. In some embodiments, the neural network includes at most two portions. In some embodiments, the first portion of the neural network includes an attention mechanism.

In some embodiments, the first portion of the neural network includes the attention mechanism that further includes an encoder architecture.

In some embodiments, the attention mechanism is applied directly to all or a portion of the input (e.g., one or more input images, in a plurality of input images for the plurality of consecutive time increments) into the model. In some embodiments, the attention mechanism is applied to an embedding of all or a portion of the input into the neural network. In some embodiments, an attention mechanism is a mapping of a query (e.g., one or more images or embeddings thereof) and a set of key-value pairs to an output where the query, keys, values, and output are all vectors. In some such embodiments, the output is computed as a weighted sum of the values, where the weight assigned to each value is computed by a compatibility function of the query with the corresponding key.

Example attention mechanisms are described in Chaudhari et al., Jul. 12, 2021 “An Attentive Survey of Attention Models,” arXiv:1904-02874v3, and Vaswani et al., “Attention is All You Need,” 31st Conference on Neural Information Processing Systems (NIPS 2017), Long Beach, California, USA, each of which is hereby incorporated by reference. The attention mechanism draws upon the inference that some portions of one or more input images, in a plurality of input images for the plurality of consecutive time increments, or any combinations thereof, are more important than others and thus some portions (elements or sets of elements) within the one or more input images (or embeddings thereof) are more important than other portions. The attention mechanism is trained to discover such importance using training images and then apply this learned (trained) observation against the one or more input images (or embeddings thereof) to form the attention embedding. Thus, the attention mechanism incorporates this notion of relevance by allowing the portion of the model downstream of the attention mechanism to dynamically pay attention to only certain parts of the input data, that help in performing the task at hand (e.g., obtaining a respective location of a respective eyelid in a respective eye) effectively.

In some embodiments, the attention mechanism is selected from the group consisting of global attention, self-attention, dot product attention, query-key-value attention, Luong attention, and Bahdanau attention.

Alternatively or additionally, in some embodiments, the neural network includes a transformer model. Non-limiting examples of transformer models contemplated for use in the present disclosure include, for example, OpenAI, ViT (Vision Transformer), BERT (Bidirectional Encoder Representations from Transformers), GPT (Generative Pre-trained Transformer), RoBERTa (Robustly Optimized BERT Pre-training), and/or T5 (Text-to-Text Transfer Transformer. See, for example, Khan et al., “Transformers in Vision: A Survey,” 2022, arXiv:2101.01169, which is hereby incorporated herein by reference in its entirety.

In some embodiments, the second portion of the neural network includes a convolutional or graph-based neural network.

In some embodiments, the first lower eyelid trace by a procedure comprising, for each respective time increment in the plurality of consecutive time increments, (i) obtaining a corresponding image of the first eye comprising a corresponding plurality of pixels and one or more pixel values for each pixel in the corresponding plurality of pixels, and (ii) inputting the corresponding image into a model, thereby obtaining the respective location of a respective eyelid (e.g., the lower eyelid and/or the upper eyelid) of the respective eye (e.g., the first eye) at the respective time increment.

In some embodiments, the model is any model disclosed herein (see, Definitions: Classifiers). In some embodiments, the model further comprises any algorithm and/or classifier disclosed herein (see, Definitions: Classifiers). For instance, in some embodiments, the model is a neural network, a support vector machine, a Naive Bayes model, a nearest neighbor model, a boosted trees model, a random forests model, a decision tree, or a clustering model.

In some embodiments, the model architecture is coded using any suitable programming language (e.g., pytorch, fastai). In some embodiments, the neural network is a pre-trained and/or transfer learned neural network (see, Definitions: Untrained Models).

### Model Training.

In some embodiments, the method includes training an untrained or partially trained neural network to generate the respective location of a respective eyelid (e.g., a lower eyelid and/or an upper eyelid) for a respective eye of a subject.

In some such embodiments, the method includes obtaining, in electronic format, a training dataset comprising, for each respective training object in a plurality of training objects, a corresponding image of an eye comprising a corresponding plurality of pixels and one or more pixel values for each pixel in the corresponding plurality of pixels. Each respective training object in the plurality of training objects in the training dataset further comprises a corresponding first measured set of coordinates that localize a first eyelid in the corresponding image. In some embodiments, an image corresponding to a respective training object in the plurality of training objects contains an upper eyelid and/or a lower eyelid. In some such embodiments, the respective training object further comprises a corresponding second measured set of coordinates that localize a second eyelid in the corresponding image.

In some embodiments, the method further includes training the untrained or partially trained neural network (e.g., a residual and/or a convolutional neural network) comprising a plurality of parameters (e.g., 10,000 or more parameters) by a procedure comprising inputting each corresponding image of each corresponding training object in the plurality of training objects as input to the untrained or partially trained neural network, thus obtaining a corresponding first calculated set of coordinates that localize the first eyelid in the corresponding image. In some embodiments, the method includes obtaining a corresponding second calculated set of coordinates that localize the second eyelid in the corresponding image. In some embodiments, the inputting the image comprises inputting an image, an image file name, and/or a file path for the image. In some embodiments, the method comprises inputting batches of images to the untrained or partially trained neural network.

In some embodiments, the plurality of training objects comprises at least 20, at least 40, at least 60, at least 80, at least 100, at least 150, at least 200, at least 250, at least 300, at least 350, at least 400, at least 450, at least 500, at least 600, at least 700, at least 800, at least 900, at least 1000, at least 2000, at least 3000, at least 4000, at least 5000, at least 6000, at least 7000, at least 8000, at least 9000, at least 10,000, at least 20,000, at least 50,000, at least 100,000, at least 200,000, at least 500,000, or at least 1 million training objects. In some embodiments, the plurality of training objects comprises no more than 2 million, no more than 500,000, no more than 100,000, no more than 50,000, no more than 20,000, no more than 10,000, no more than 5000, no more than 2000, no more than 1000, or no more than 500 training objects. In some embodiments, the plurality of training objects comprises from 2 to 100,000, from 100 to 500,000, from 10 to 5000, from 10,000 to 50,000, from 100,000 to 1 million, or from 1 million to 2 million training objects. In some embodiments, the plurality of training objects comprises a different range starting no lower than 20 training objects and ending no higher than 2 million training objects.

In some embodiments, the training the untrained or partially trained neural network further comprises using at least a difference between the corresponding first calculated set of coordinates and the corresponding first measured set of coordinates obtained for each object in the plurality of objects to update all or a subset of the plurality of parameters (e.g., 10,000 or more parameters), thereby training the neural network to localize the first eyelid in an image. In some embodiments, the training the untrained or partially trained neural network further includes using at least a difference between the corresponding second calculated set of coordinates and the corresponding second measured set of coordinates obtained for each object in the plurality of objects to update all or a subset of the plurality of parameters (e.g., 10,000 or more parameters), thereby training the neural network to localize the second eyelid in an image.

Generally, training a classifier (e.g., a neural network) comprises updating the plurality of parameters (e.g., weights) for the respective classifier through backpropagation (e.g., gradient descent). First, a forward propagation is performed, in which input data (e.g., a corresponding image for each respective training object in a plurality of training objects in the training dataset) is accepted into the neural network, and an output is calculated based on the selected activation function and an initial set of parameters (e.g., weights and/or hyperparameters). In some embodiments, parameters (e.g., weights and/or hyperparameters) are randomly assigned (e.g., initialized) for the untrained or partially trained neural network. In some embodiments, parameters are transferred from a previously saved plurality of parameters or from a pre-trained model (e.g., by transfer learning).

A backward pass is then performed by calculating an error gradient for each respective parameter corresponding to each respective unit in each layer, where the error for each parameter is determined by calculating a loss (e.g., error) based on the network output (e.g., the predicted value) and the input data (e.g., the expected value or true labels). Parameters (e.g., weights) are then updated by adjusting the value based on the calculated loss, thereby training the neural network.

For example, in some general embodiments of machine learning, backpropagation is a method of training a network with hidden layers comprising a plurality of weights (e.g., embeddings). The output of an untrained model (e.g., the calculated set of coordinates for an eyelid generated by a neural network) is first generated using a set of arbitrarily selected initial weights. The output is then compared with the original input (e.g., the measured set of coordinates for the eyelid obtained from the training dataset) by evaluating an error function to compute an error (e.g., using a loss function). The weights are then updated such that the error is minimized (e.g., according to the loss function). In some embodiments, any one of a variety of backpropagation algorithms and/or methods are used to update the plurality of weights, as will be apparent to one skilled in the art.

In some embodiments, the loss function is mean square error, quadratic loss, mean absolute error, mean bias error, hinge, multi-class support vector machine, and/or cross-entropy. In some embodiments, training the untrained or partially trained neural network comprises computing an error in accordance with a gradient descent algorithm and/or a minimization function.

In some embodiments, the error function is used to update one or more parameters (e.g., weights) in a neural network by adjusting the value of the one or more parameters by an amount proportional to the calculated loss, thereby training the neural network. In some embodiments, the amount by which the parameters are adjusted is metered by a learning rate hyperparameter that dictates the degree or severity to which parameters are updated (e.g., smaller or larger adjustments). Thus, in some embodiments, the training updates all or a subset of the plurality of parameters (e.g., 10,000 or more parameters) based on a learning rate. In some embodiments, the learning rate is a differential learning rate.

In some embodiments, the training further uses a regularization on the corresponding parameter of each hidden neuron in the corresponding plurality of hidden neurons. For example, in some embodiments, a regularization is performed by adding a penalty to the loss function, where the penalty is proportional to the values of the parameters in the trained or untrained neural network. Generally, regularization reduces the complexity of the model by adding a penalty to one or more parameters to decrease the importance of the respective hidden neurons associated with those parameters. Such practice can result in a more generalized model and reduce overfitting of the data. In some embodiments, the regularization includes an L1 or L2 penalty. For example, in some preferred embodiments, the regularization includes an L2 penalty on lower and upper parameters. In some embodiments, the regularization comprises spatial regularization or dropout regularization. In some embodiments, the regularization comprises penalties that are independently optimized.

In some embodiments, the training process is repeated for each learning instance in a plurality of learning instances. For example, the inputting each corresponding image of each corresponding training object in the plurality of training objects as input to the untrained or partially trained neural network thereby obtaining a corresponding first calculated set of coordinates that localize an eyelid in the corresponding image, and the using at least a difference between the corresponding calculated set of coordinates and the corresponding first measured set of coordinates obtained for each object in the plurality of objects to update all or a subset of the plurality of parameters (e.g., 10,000 or more parameters) is repeated for each of a plurality of learning instances, thus training the neural network to localize an eyelid (e.g., an upper eyelid and/or a lower eyelid) in an image.

In some embodiments, the plurality of learning instances comprises at least 3, at least 4, at least 5, at least 6, at least 7, at least 8, at least 9, at least 10, at least 50, at least 100, at least 500, at least 1000, at least 2000, at least 3000, at least 4000, at least 5000, or at least 7500 learning instances. In some embodiments, the plurality of learning instances comprises no more than 10,000, no more than 5000, no more than 1000, no more than 500, no more than 100, or no more than 50 learning instances. In some embodiments, the plurality of learning instances comprises from 3 to 10, from 5 to 100, from 100 to 5000, or from 1000 to 10,000 learning instances. In some embodiments, the plurality of learning instances falls within another range starting no lower than 3 learning instances and ending no higher than 10,000 learning instances.

In some such embodiments, the training includes repeating the adjustment of the parameters of the neural network (e.g., via backpropagation) over a plurality of instances, therefore increasing the neural network's accuracy in calculating coordinates localizing an eyelid.

In some implementations, the training process is repeated for each training epoch in a respective plurality of training epochs, in a respective learning instance in the plurality of learning instances. Thus, in some embodiments, the training occurs over a plurality of training loops (e.g., learning instances), where, for one or more training loops in the plurality of training loops, the training occurs over plurality of epochs. In some embodiments, the number of epochs is a hyperparameter.

In some embodiments, parameters can differ between training instances and/or between epochs within a respective training instance. For example, a learning rate can be modulated (e.g., can be slower or faster) between different training instances, different epochs of a respective training instance, and/or different layers of the neural network architecture within a respective training epoch. In another example, a number of epochs can be changed between a first training instance and a second training instance.

In some embodiments, parameters are held constant between training instances and/or between epochs within a respective training instance. For instance, in some embodiments, the training further comprises, for a respective learning instance in the plurality of learning instances, freezing one or more of the plurality of parameters (e.g., 10,000 or more parameters) prior to the training.

In some embodiments, one or more parameters are frozen or unfrozen. In some embodiments, all of the parameters within a respective layer in a plurality of layers and/or within a respective block in a plurality of blocks are frozen or unfrozen. In some embodiments, all of the parameters across all of the layers in the neural network are frozen or unfrozen. In some embodiments, all of the parameters across all of the layers in the neural network except for the regressor layer are frozen.

In some embodiments, the plurality of parameters that are frozen varies between learning instances and/or between training epochs within a respective learning instance. In an example embodiment, the training process further comprises increasing the number of layers that are frozen after each successive learning instance in a plurality of learning instances, starting with the uppermost layers and gradually working towards the deeper layers.

In some embodiments, the training comprises transfer learning. Transfer learning is further described, for example, in the Definitions section (see, “Untrained models,” above).

In some embodiments, training the untrained or partially trained neural network forms a trained neural network following a first evaluation of an error function. In some such embodiments, training the untrained or partially trained neural network forms a trained neural network following a first updating of one or more parameters based on a first evaluation of an error function. In some alternative embodiments, training the untrained or partially trained neural network forms a trained neural network following at least 1, at least 2, at least 3, at least 4, at least 5, at least 6, at least 7, at least 8, at least 9, at least 10, at least 20, at least 30, at least 40, at least 50, at least 100, at least 500, at least 1000, at least 10,000, at least 50,000, at least 100,000, at least 200,000, at least 500,000, or at least 1 million evaluations of an error function. In some such embodiments, training the untrained or partially trained neural network forms a trained neural network following at least 1, at least 2, at least 3, at least 4, at least 5, at least 6, at least 7, at least 8, at least 9, at least 10, at least 20, at least 30, at least 40, at least 50, at least 100, at least 500, at least 1000, at least 10,000, at least 50,000, at least 100,000, at least 200,000, at least 500,000, or at least 1 million updatings of one or more parameters based on the at least 1, at least 2, at least 3, at least 4, at least 5, at least 6, at least 7, at least 8, at least 9, at least 10, at least 20, at least 30, at least 40, at least 50, at least 100, at least 500, at least 1000, at least 10,000, at least 50,000, at least 100,000, at least 200,000, at least 500,000, or at least 1 million evaluations of an error function.

In some embodiments, training the untrained or partially trained neural network forms a trained neural network when the neural network satisfies a minimum performance requirement. For example, in some embodiments, training the untrained or partially trained neural network forms a trained neural network when the error calculated for the trained neural network, following an evaluation of an error function across one or more training datasets for a respective one or more training objects, satisfies an error threshold. In some embodiments, the error calculated by the error function across one or more training datasets for a respective one or more training objects satisfies an error threshold when the error is less than 20 percent, less than 18 percent, less than 15 percent, less than 10 percent, less than 5 percent, or less than 3 percent. Thus, for example, in some embodiments, a trained neural network is formed when the best performance is achieved (e.g., in some instances, a trained neural network is selected from an earlier training instance rather than a later training instance, if the earlier training instance resulted in better performance than the later training instance).

In some embodiments, neural network performance is measured using a training loss metric, a validation loss metric, and/or a mean absolute error. For instance, in some embodiments, neural network performance is measured by validating the model using one or more images in a validation dataset and determined based at least on a difference between a corresponding calculated set of coordinates and the corresponding first measured set of coordinates obtained for each image in the one or more images in the validation dataset. In some such embodiments, training the untrained or partially trained neural network forms a trained neural network when the neural network satisfies a minimum performance requirement based on a validation training.

In some embodiments, the method comprises any suitable method for validation, including but not limited to K-fold cross-validation, advanced cross-validation, random cross-validation, grouped cross-validation (e.g., K-fold grouped cross-validation), bootstrap bias corrected cross-validation, random search, and/or Bayesian hyperparameter optimization.

Suitable embodiments for obtaining eyelid traces, including images, data preprocessing, keypoints, model architecture, parameters, model training and/or methods of performing or obtaining the same, include any of the embodiments disclosed in U.S. Provisional Application No. 63/194,554, filed May 28, 2021 and/or U.S. Provisional Application No. 63/275,749, filed Nov. 4, 2021, each of which is hereby incorporated herein by reference in its entirety for all purposes, and any substitutions, modifications, additions, deletions, and/or combinations thereof as will be apparent to one skilled in the art.

### Example Applications.

In some embodiments, the method includes using a determination of eye closure to obtain a first measurement of a characteristic of a blink response (e.g., an involuntary blink response) in the subject (e.g., for a subject suspected of having a neurological condition).

In some embodiments, the characteristic of the blink response is selected from the group consisting of individual latency, differential latency, number of oscillations, change in tonic lid position, horizontal lid velocity (e.g., for an upper and lower lid), vertical lid velocity (e.g., for an upper and lower lid), time to close, time to open, total blink time, and time under threshold.

Tonic lid position refers to a moving average of the pixel location of the upper eyelid when not in a blink. Threshold is defined as a number of pixels (e.g., 20 pixels) below tonic lid position. Latency refers to the time differential between stimulation and ipsilateral eye (e.g., the stimulated eye) movement. Differential latency refers to the time differential between the start of ipsilateral eye movement and the start of contralateral eye (e.g., the eye opposite the stimulation) movement. Lid excursion refers to the distance traveled by the eyelid from the tonic lid position to closed position, measured in pixels. Lid velocity refers to the average eyelid speed (e.g., in pixels per second) in a first plurality of frames (e.g., 7 frames) following start of eyelid movement. Time to close refers to the time for an eyelid to travel from tonic lid position to the closed position. Time to open refers to the time for an eyelid to travel from closed position back to tonic lid position. Total blink time refers to the time from start of eyelid movement until it returns to its tonic lid position. Time under threshold refers to the time that the eyelid spends below the threshold position. Number of oscillations is defined as the number of cycles of up and down upper eyelid movement after a stimulated blink. Delta 30 refers to the time difference between the ipsilateral eye and contralateral eye after the lids had moved a threshold number of pixels (e.g., 30 pixels) from the tonic lid position.

In some embodiments, the characteristic of the blink response is a number of times the subject blinks within the plurality of consecutive time increments. In some embodiments, the plurality of consecutive time increments is at least 1 second (s), at least 5 s, at least 10 s, at least 15 s, at least 20 s, at least 25 s, at least 30 s, at least 35 s, at least 40 s, at least 45 s, at least 50 s, at least 55 s, at least 1 minute (min), at least 2 min, at least 3 min, at least 4 min, at least 5 min, at least 6 min, at least 7 min, at least 8 min, at least 9 min, or at least 10 min. In some embodiments, the plurality of consecutive time increments is between 1 second and 30 minutes.

Characteristics of blink responses are described in further detail, for example, in Garner et al., 2018, “Blink reflex parameters in baseline, active, and head-impact Division I athletes,” Cogent Engineering, 5:1429110; doi: 10.1080/23311916.2018.1429110, which is hereby incorporated herein by reference in its entirety.

In some implementations, the method includes using a respective eyelid trace and a known image capture rate (e.g., a frame rate) for the respective eyelid trace to calculate the various metrics for eyelid movement as a function of time.

In some embodiments, the method includes using a determination of eye closure to obtain a second measurement of the characteristic of the blink response for the subject, where the second measurement represents a baseline condition for the subject. The method includes comparing the first measurement and the second measurement, and when the difference between the first measurement and the second measurement exceeds a predetermined threshold value, determining that the subject is afflicted with a neurological condition.

For example, in some embodiments, the baseline condition is obtained from the subject during a period of healthy and/or normal activity. In some embodiments, the baseline condition is obtained from the subject prior to onset of a suspected neurological condition. In some embodiments, the baseline condition is obtained from a healthy control subject.

Methods for measuring blink responses and determining whether a subject is afflicted with a neurological condition are disclosed in further detail in U.S. patent application Ser. No. 14/787,564, filed May 1, 2014, International Patent Application No. PCT/US2018/032666, having an International Filing Date of May 15, 2018, U.S. Provisional Application No. 63/194,554, filed May 28, 2021, and/or U.S. Provisional Application No. 63/275,749, filed Nov. 4, 2021, each of which is hereby incorporated herein by reference in its entirety for all purposes.

### Additional Embodiments

Another aspect of the present disclosure provides a computing system, comprising one or more processors and memory storing one or more programs to be executed by the one or more processor, the one or more programs comprising instructions for a method of determining an eye closure status of a respective subject. The method comprises obtaining, in electronic format, a first lower eyelid trace, where the first lower eyelid trace comprises, for each respective time increment in a plurality of consecutive time increments, a respective location of a lower eyelid of a first eye of the respective subject. The method further comprises obtaining, in electronic format, a first upper eyelid trace, where the first upper eyelid trace comprises, for each respective time increment in the plurality of consecutive time increments, a respective location of an upper eyelid of the first eye.

The method includes obtaining, between a first and second time increment within the plurality of consecutive time increments, a first minimum difference between the location of the upper eyelid and the lower eyelid of the first eye across the time increments in the plurality of consecutive time increments that are between the first and second time increment. The method further includes obtaining, between the first and second time increment within the plurality of consecutive time increments, a first maximum difference between the location of the upper eyelid and the lower eyelid of the first eye across the time increments in the plurality of consecutive time increments that are between the first and second time increment. The first minimum difference and a difference between the first maximum difference and the first minimum difference is passed through an activation function thereby obtaining a first result, and the first result is used to provide an eye closure status of the first eye.

Another aspect of the present disclosure provides a non-transitory computer readable storage medium storing one or more programs for training a neural network to determine an eye closure status of a respective subject, the one or more programs configured for execution by a computer. The one or more programs comprise instructions for obtaining, in electronic format, a first lower eyelid trace, where the first lower eyelid trace comprises, for each respective time increment in a plurality of consecutive time increments, a respective location of a lower eyelid of a first eye of the respective subject. The instructions further comprise obtaining, in electronic format, a first upper eyelid trace, where the first upper eyelid trace comprises, for each respective time increment in the plurality of consecutive time increments, a respective location of an upper eyelid of the first eye.

The instructions include obtaining, between a first and second time increment within the plurality of consecutive time increments, a first minimum difference between the location of the upper eyelid and the lower eyelid of the first eye across the time increments in the plurality of consecutive time increments that are between the first and second time increment. The instructions further include obtaining, between the first and second time increment within the plurality of consecutive time increments, a first maximum difference between the location of the upper eyelid and the lower eyelid of the first eye across the time increments in the plurality of consecutive time increments that are between the first and second time increment. The first minimum difference and a difference between the first maximum difference and the first minimum difference is passed through an activation function thereby obtaining a first result, and the first result is used to provide an eye closure status of the first eye.

Still another aspect of the present disclosure provides a computing system including the above-disclosed one or more processors and memory storing one or more programs that further comprise instructions for performing any of the above-disclosed methods alone or in combination.

Another aspect of the present disclosure provides non-transitory computer-readable storage medium comprising the above-disclosed one or more programs in which the one or more programs further comprise instructions for performing any of the above-disclosed methods alone or in combination. The one or more programs are configured for execution by a computer.

### EXAMPLES

### Example 1—Performance of Residual Convolutional Neural Network Architecture Versus Pre-Trained ResNet

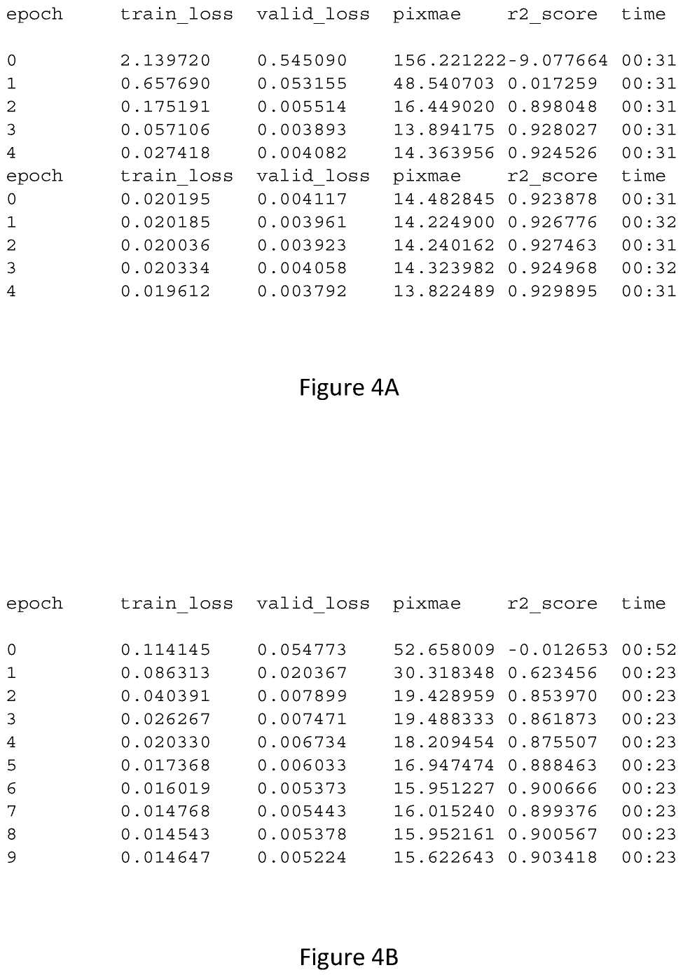

FIGS. 4A-B illustrate example performance measures of a transfer-learned ResNet model (FIG. 4A) and a residual convolutional neural network with custom architecture trained in accordance with some embodiments of the present disclosure (FIG. 4B).

A pre-trained ResNet model was trained, using transfer learning, on a training dataset including a plurality of images. The model's architecture included the use of a ReLU activation function, the Ranger optimizer, and flat cosine-annealed training. Training was performed for 10 epochs (in two sets of 5 epochs labeled epochs 0-4). For the first 5 epochs, parameters in all layers except the final layer were frozen, and only the final layer was trained. For the final 5 epochs, all layers were unfrozen, and every layer of the model was trained. The model was validated using a validation dataset including a subset of images not used during training. The ability of the model to correctly identify coordinates for eyelid localization was measured using training loss, validation loss, r2 scores (e.g., where values closer to 1 indicate greater accuracy of predicted values compared to actual values), and mean absolute error of predictions in pixels (pixmae). Completion time (speed) of each epoch was also measured. As illustrated in FIG. 4A, training loss, validation loss, and mean absolute error decreased sharply during the first 5 epochs and remained relatively constant for the final 5 epochs. Similarly, r2 scores increased sharply during the first 5 epochs and remained relatively constant for the final 5 epochs. These performance measures indicated the accuracy of the model. However, the pre-trained ResNet model's bloat and lack of domain specificity indicated that a custom architecture was the optimal approach.

A custom ResNet architecture (e.g., a residual convolutional neural network) model was constructed in accordance with some embodiments of the present disclosure. A Mish activation function was used instead of ReLU and the QHAdam optimizer was used instead of Ranger. Flat cosine-annealed training on every layer of the model was performed for 10 epochs on the training dataset and validated using the validation dataset described above. FIG. 4B shows the report from the final epochs of training (pictured as epochs 0-9), including training loss, validation loss, r2 score, mean absolute error of predictions in pixels, and completion time. Accuracy of the custom architecture was comparable to that of the pre-trained ResNet model, but the completion time for each epoch was shorter, indicating that the systems and methods provided herein improved the speed of computation. The shallowness of the architecture provides greater efficiency and consistency given the specificity of the domain in the input images. As provided in the present disclosure, various combinations are possible for the configuration of the architecture with regards to the optimizer, activation function, network depth, convolution kernel sizes, capabilities for generalization, self-attention, and optical flow estimation, among other specifications.

### Example 2—Performance of Trained Auxiliary Neural Network for Determining a Class of an Image

FIGS. 8A-D illustrate example outputs of an auxiliary neural network for determining a class of an image. A plurality of pairs of training objects was obtained from a plurality of training objects in a training dataset used for training a neural network to localize an eyelid in an image, as in Example 1 above. The subject identity for each respective image in each pair of images for the corresponding pair of training objects was determined using an annotation (e.g., a numerical identifier included in the filename) for the respective image. In addition, the annotation for each respective image included a scan identity, the capture orientation (e.g., left or right eye), and a frame number for the respective image. An auxiliary neural network using a pre-trained ResNet18 model architecture was trained, using transfer learning, as a binary classifier to generate similarity predictions (e.g., true or false), using, as input, each respective pair of images in the plurality of pairs of training objects and a similarity label for the respective pair of images derived from a comparison of the subject identifiers for each respective image in the respective pair of images.

Outputs from the trained auxiliary neural network were assessed using a validation dataset to determine the models ability to predict whether or not two respective images of an eye are obtained from the same subject or from different subjects. Notably, the trained auxiliary neural network achieved 99.1% accuracy on the validation set. FIGS. 8A and 8D illustrate the model's ability to differentiate between images obtained from different subjects (e.g., “Not similar”). FIGS. 8B and 8C illustrate the model's ability to accurately identify images obtained from the same subject (e.g., “Similar”), regardless of different eyelid orientations (e.g., FIG. 8C: left panel: open; right panel: closed) and/or textures (e.g., FIG. 8B: left panel: eyelid with makeup; right panel: eyelid without makeup).

### Example 3—Determination of Eye Closure Status Using Involuntary Eye Stimulus

Eyelid traces were used to determine eye closure status, in accordance with some embodiments of the present disclosure.

Table 1 presents sample data obtained from an eyelid trace generated using a sequence of 8 involuntary eye stimuli. The eyelid trace included eyelid locations obtained for each respective eyelid (e.g., upper and lower), for each respective eye (e.g., left and right) of a subject. In Table 1, excerpts of the eyelid trace including the first two stimuli are shown, where time intervals are recorded in the first column (“Time”) in milliseconds and stimuli are recorded in the second column (“Stimulus”) as “MC.” At specific time intervals in the plurality of time intervals, stimuli were directed to either the left eye (MC-OS; “oculus sinister”) or the right eye (MC-OD; “oculus dexter”). Eyelid locations for each of the right eye upper eyelid, left eye upper eyelid, right eye lower eyelid, and left eye lower eyelid are indicated in the remaining columns.

TABLE 1
Eyelid trace.

| Time | | Right | Left | Right | Left |
|---|---|---|---|---|---|
| (msec) | Stimulus | Top | Top | Bottom | Bottom |
| 0 | None | 161 | 193 | 156 | 188 |
| 4 | None | 161 | 193 | 155 | 189 |
| 7 | None | 161 | 194 | 155 | 189 |
| 11 | None | 160 | 194 | 154 | 189 |
| 14 | None | 160 | 194 | 154 | 190 |
| 18 | None | 160 | 195 | 153 | 190 |
| 21 | None | 160 | 196 | 154 | 191 |
| 25 | None | 160 | 197 | 153 | 193 |
| 29 | TA-OD | 160 | 198 | 153 | 194 |
| 32 | None | 161 | 199 | 154 | 195 |
| 36 | MC-OD | 162 | 201 | 155 | 196 |
| 39 | None | 163 | 204 | 156 | 199 |
| 43 | None | 165 | 206 | 158 | 201 |
| 46 | None | 166 | 209 | 159 | 203 |
| 50 | None | 168 | 214 | 162 | 207 |
| . . . | . . . | . . . | . . . | . . . | . . . |
| 3039 | None | 306 | 340 | 181 | 207 |
| 3043 | None | 306 | 340 | 180 | 207 |
| 3046 | None | 306 | 340 | 180 | 207 |
| 3050 | None | 306 | 340 | 180 | 207 |
| 3054 | None | 306 | 340 | 180 | 207 |
| 3057 | None | 306 | 340 | 180 | 207 |
| 3061 | TA-OS | 306 | 340 | 180 | 207 |
| 3064 | None | 306 | 340 | 180 | 207 |
| 3068 | MC-OS | 306 | 341 | 180 | 207 |
| 3071 | None | 306 | 341 | 180 | 207 |
| 3075 | None | 306 | 341 | 180 | 208 |
| 3079 | None | 306 | 340 | 180 | 208 |
| 3082 | None | 306 | 340 | 180 | 208 |
| 3086 | None | 306 | 340 | 180 | 207 |
| 3089 | None | 306 | 340 | 180 | 207 |
| . . . | . . . | . . . | . . . | . . . | . . . |

For each respective stimulus in the plurality of stimuli, a time window was determined including a predetermined amount of time prior to the involuntary eye stimulus and a second predetermined amount of time after the involuntary eye stimulus. For each respective stimulus, for each respective eye, a respective minimum difference between the location of the upper eyelid and the location of the lower eyelid were obtained across the respective time window. Further, for each respective stimulus, for each respective eye, a respective maximum difference between the location of the upper eyelid and the location of the lower eyelid was obtained across the respective time window. Thus, a minimum difference for the right eye, a maximum difference for the right eye, a minimum difference for the left eye, and a maximum difference for the left eye were obtained for each respective stimulus.

For each respective stimulus in the plurality of stimuli, for each respective eye, a respective result was obtained by passing a square of the respective minimum difference divided by a difference between the respective maximum difference and the respective minimum difference through an activation function (e.g., a sigmoid function). The respective result was used to provide an eye closure status of the respective eye, where, when the respective result was less than a threshold of 0.92, then the eye was deemed to have experienced a blink, and when the respective result was greater than or equal to a threshold of 0.92, then the eye was deemed not to have experienced a blink.

Table 2 indicates the eye closure status of each respective eye for each respective stimulus in the plurality of stimuli. For each stimulus (“Stim Index”), the eye to which the stimulus was directed is indicated (“Stim Side”). Closure status is then indicated for the eye to which the stimulus was directed (ipsilateral eye; “Ipsi”) as well as for the contralateral eye (“Contra”). For instance, the closure status reports “TRUE” when a respective eye was deemed to have experienced a blink, and the closure status reports “FALSE” when the respective eye was deemed not to have experienced a blink.

TABLE 2
Eye closure status.

| Stim Index | Stim Side | Ipsi | Contra |
|---|---|---|---|
| 1 | R | FALSE | TRUE |
| 2 | L | TRUE | FALSE |
| 3 | R | TRUE | FALSE |
| 4 | L | FALSE | FALSE |
| 5 | L | FALSE | FALSE |
| 6 | R | TRUE | FALSE |
| 7 | R | TRUE | TRUE |
| 8 | L | TRUE | TRUE |

These results demonstrate a method for automated detection of eye closure status using eyelid traces, such as those obtained from high-speed image capture and tracking, via machine learning, of eyelid position and movement.

### CONCLUSION

The terminology used herein is for the purpose of describing particular cases and is not intended to be limiting. As used herein, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

Plural instances may be provided for components, operations or structures described herein as a single instance. Finally, boundaries between various components, operations, and data stores are somewhat arbitrary, and particular operations are illustrated in the context of specific illustrative configurations. Other allocations of functionality are envisioned and may fall within the scope of the implementation(s). In general, structures and functionality presented as separate components in the example configurations may be implemented as a combined structure or component. Similarly, structures and functionality presented as a single component may be implemented as separate components. These and other variations, modifications, additions, and improvements fall within the scope of the implementation(s).

It will also be understood that, although the terms first, second, etc. may be used herein to describe various elements, these elements should not be limited by these terms. These terms are used to distinguish one element from another. For example, a first subject could be termed a second subject, and, similarly, a second subject could be termed a first subject, without departing from the scope of the present disclosure. The first subject and the second subject are both subjects, but they are not the same subject.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event)” or “in response to detecting (the stated condition or event),” depending on the context.

The foregoing description included example systems, methods, techniques, instruction sequences, and computing machine program products that embody illustrative implementations. For purposes of explanation, numerous specific details were set forth in order to provide an understanding of various implementations of the inventive subject matter. It will be evident, however, to those skilled in the art that implementations of the inventive subject matter may be practiced without these specific details. In general, well-known instruction instances, protocols, structures and techniques have not been shown in detail.

The foregoing description, for purpose of explanation, has been described with reference to specific implementations. However, the illustrative discussions above are not intended to be exhaustive or to limit the implementations to the precise forms disclosed. Many alterations, modifications, and variations will be apparent to those skilled in the art in light of the foregoing description without departing from the spirit or scope of the present disclosure and that when numerical lower limits and numerical upper limits are listed herein, ranges from any lower limit to any upper limit are contemplated. The implementations were chosen and described in order to best explain the principles and their practical applications, to thereby enable others skilled in the art to best utilize the implementations and various implementations with various modifications as are suited to the particular use contemplated.

What is claimed:

1. A method for determining an eye closure status of a respective subject, the method comprising:
(a) obtaining, in electronic format, a first lower eyelid trace, wherein the first lower eyelid trace comprises, for each respective time increment in a plurality of consecutive time increments, a respective location of a lower eyelid of a first eye of the respective subject;
(b) obtaining, in electronic format, a first upper eyelid trace, wherein the first upper eyelid trace comprises, for each respective time increment in the plurality of consecutive time increments, a respective location of an upper eyelid of the first eye;
(c) obtaining, between a first and second time increment within the plurality of consecutive time increments, a first minimum difference between the location of the upper eyelid and the lower eyelid of the first eye across the time increments in the plurality of consecutive time increments that are between the first and second time increment;
(d) obtaining, between the first and second time increment within the plurality of consecutive time increments, a first maximum difference between the location of the upper eyelid and the lower eyelid of the first eye across the time increments in the plurality of consecutive time increments that are between the first and second time increment;
(e) passing (i) the first minimum difference and (ii) a difference between the first maximum difference and the first minimum difference through an activation function thereby obtaining a first result; and
(f) using the first result to provide an eye closure status of the first eye.

2. The method of claim 1, wherein the eye closure status of the first eye is a first Boolean status indicator of whether or not the first eye experienced an eye blink at any point between the first and second time increment wherein
the first eye is deemed to have experienced an eye blink at a point between the first and second time increment when the first result satisfies a first threshold, and
the first eye is deemed to have not experienced an eye blink at any point between the first and second time increment when the first result fails to satisfy the first threshold.

3. The method of claim 1 or 2, wherein the threshold is between 0.80 and 0.97.

4. The method of claim 1 or 2, wherein the threshold is between 0.89 and 0.95.

5. The method of any one of claims 1-4, wherein the first and second time increment are between 50 milliseconds and 500 milliseconds apart from each other.

6. The method of any one of claims 1-5, the method further comprising:
(g) obtaining, in electronic format, a second lower eyelid trace, wherein the second lower eyelid trace comprises, for each respective time increment in the plurality of consecutive time increments, a respective location of a lower eyelid of a second eye of the respective subject;
(h) obtaining, in electronic format, a second upper eyelid trace, wherein the second upper eyelid trace comprises, for each respective time increment in the plurality of consecutive time increments, a respective location of an upper eyelid of the second eye;
(i) obtaining, between a first and second time increment within the plurality of consecutive time increments, a second minimum difference between the location of the upper eyelid and the lower eyelid of the second eye across the time increments in the plurality of consecutive time increments that are between the first and second time increment;
(j) obtaining, between the first and second time increment within the plurality of consecutive time increments, a second maximum difference between the location of the upper eyelid and the lower eyelid of the second eye across the time increments in the plurality of consecutive time increments that are between the first and second time increment;
(k) passing (i) the second minimum difference and (ii) a difference between the second maximum difference and the second minimum difference through the activation function thereby obtaining a second result; and
(l) using the second result to provide an eye closure status of the second eye.

7. The method of claim 6, wherein an involuntary eye stimulus occurs at a time point between the first and second time increment, and wherein the first time increment is a first predetermined amount of time prior to the involuntary eye stimulus and the second time increment is a second predetermined amount of time after the involuntary eye stimulus.

8. The method of claim 7, wherein
the first predetermined amount of time is between 5 milliseconds and 30 milliseconds, and
the second predetermined amount of time is between 75 milliseconds and 150 milliseconds.

9. The method of claim 6, wherein
the involuntary eye stimulus is directed to the first eye or the second eye and the method further comprises reporting out whether the involuntary eye stimulus is directed to the first eye or the second eye along with an indication as to whether the involuntary eye stimulus was directed to the first eye or the second eye.

10. The method of claim 9, the method further comprising, prior to the obtaining (a), generating the involuntary eye stimulus.

11. The method of claim 10, wherein the involuntary eye stimulus is a puff of air directed to the first eye or the second eye.

12. The method of claim 10, wherein the involuntary eye stimulus is a flash of light directed to the first eye or the second eye.

13. The method any one of claims 1-12, the method further comprising repeating the obtaining (a), the obtaining (b), the obtaining (c), the obtaining (d), the passing (e), and the using (f), for each respective subject in a plurality of subjects.

14. The method of claim 13, wherein the plurality of subjects is 50 or more subjects, 100 or more subjects, 1000 or more subjects, 10,000 or more subjects, or 100,000 or more subjects.

15. The method of any one of claims 1-14, wherein the activation function normalizes the first result to a value between 0 and 1.

16. The method of claim 15, wherein the activation function is a logistic sigmoid function.

17. The method of any one of claims 1-16, wherein the plurality of consecutive time increments consists of between 20 time increments and 1000 time increments.

18. The method of any one of claims 1-17, wherein each time increment in the plurality of consecutive time increments represents between 1 millisecond and 10 milliseconds of time.

19. The method of any one of claims 1-18, the method further comprising generating the first lower eyelid trace by a procedure comprising:
for each respective time increment in the plurality of consecutive time increments:
(i) obtaining a corresponding image of the first eye comprising a corresponding plurality of pixels and one or more pixel values for each pixel in the corresponding plurality of pixels, and
(ii) inputting the corresponding image into a trained neural network comprising 10,000 or more parameters, thereby obtaining the respective location of the lower eyelid of the first eye at the respective time increment.

20. The method claim 19, wherein the neural network comprises:
a plurality of convolutional layers, wherein each convolutional layer in the plurality of convolutional layers comprises one or more filters, a respective size, and a respective stride; and
one or more pooling layers, wherein each pooling layer in the one or more pooling layers comprises a respective size and a respective stride.

21. The method of claim 19, wherein the neural network is LeNet, AlexNet, VGGNet 16, GoogLeNet, ResNet, SE-ResNeXt, MobileNet, or EfficientNet.

22. The method of claim 19, wherein an edge length, in pixels, of the corresponding image consists of between 164 pixels and 1024 pixels.

23. The method of claim 19, wherein the neural network comprises:
an initial convolutional neural network layer that receives a grey-scaled pixel value for each pixel in the corresponding plurality of pixels as input into the neural network, wherein the initial convolutional neural network layer includes a first activation function, and wherein the initial convolutional neural network layer convolves the corresponding plurality of pixels into more than 10 separate parameters for each pixel in the corresponding plurality of pixels.

24. The method of claim 23, wherein the neural network further comprises a pooling layer that pools the 10 separate parameters for each pixel in the plurality of pixels outputted by the initial convolutional neural network layer.

25. The method of claim 23, wherein the initial convolutional neural network layer has a stride of two or more.

26. The method of claim 24, wherein the neural network further comprises a plurality of intermediate blocks including a first intermediate block and a final intermediate block, wherein
the first intermediate block takes as input the output of the pooling layer,
each intermediate block in the plurality of intermediate blocks other than the first intermediate block and the final intermediate block takes, as input, an output of another intermediate block in the plurality of intermediate blocks and has an output that serves as input to another intermediate block in the plurality of intermediate blocks, and
each intermediate block comprises a respective first convolutional layer comprising more than 1000 parameters, wherein the respective convolutional layer has a corresponding activation function.

27. The method of claim 26, wherein each intermediate block in the plurality of intermediate blocks comprises a corresponding second convolutional layer that takes, as input, an output of the respective first convolutional layer.

28. The method of claim 27, wherein each intermediate block in the plurality of intermediate blocks comprises a merge layer that merges (i) an output of the respective second convolutional layer and (ii) an output of a preceding intermediate block in the plurality of intermediate blocks.

29. The method of claim 28, wherein:
each intermediate block in the plurality of intermediate blocks has a corresponding input size and a corresponding output size, and,
when the corresponding input size of a respective intermediate block differs from the corresponding output size, the respective intermediate block further comprises a corresponding third convolutional layer that receives, as input, the (ii) output of the preceding intermediate block, wherein the corresponding third convolutional layer convolves the (ii) output of the preceding intermediate block prior to the merging (i) and (ii) by the merge layer.

30. The method of claim 26, wherein the final intermediate block takes, as input, an output of another intermediate block in the plurality of intermediate blocks and produces, as output, a flattened data structure comprising a predetermined plurality of values.

31. The method of claim 30, wherein the neural network further comprises a regressor block including a first dropout layer, a first linear layer, and a corresponding activation function, wherein the regressor block takes, as input, the flattened data structure comprising the predetermined plurality of values.

32. The method of claim 31, wherein the first dropout layer removes a first subset of values from the plurality of values in the flattened data structure, based on a first dropout rate.

33. The method of claim 31 or 32, wherein the first linear layer applies a first linear transformation to the plurality of values in the flattened data structure.

34. The method of any one of claims 31-33, wherein the regressor block further includes a second dropout layer, wherein the second dropout layer removes a second subset of values from the plurality of values in the flattened data structure, based on a second dropout rate.

35. The method of any one of claims 31-34, wherein the regressor block further includes a second linear layer, wherein the second linear layer applies a second linear transformation to the plurality of values in the flattened data structure.

36. The method of any one of claims 23-35, wherein the first activation function is tanh, sigmoid, softmax, Gaussian, Boltzmann-weighted averaging, absolute value, linear, rectified linear unit (ReLU), bounded rectified linear, soft rectified linear, parameterized rectified linear, average, max, min, sign, square, square root, multiquadric, inverse quadratic, inverse multiquadric, polyharmonic spline, swish, mish, Gaussian error linear unit (GeLU), scaled exponential linear unit (SELU), or thin plate spline.

37. The method of any one of claims 31-36, wherein the regressor block produces, as output, a corresponding first calculated set of coordinates that localize the lower eyelid in the corresponding image.

38. The method of any one of claims 1-37, the method further comprising using the eye closure status of the first eye to diagnose a condition of the respective subject.

39. The method of claim 38, wherein the condition is a neurological condition.

40. The method of claim 39, wherein the condition is Parkinson's disease, Huntington's disease, schizophrenia, or a traumatic brain injury.

41. The method of claim 38, wherein the condition is Alzheimer's disease.

42. The method of claim 38, wherein the condition is a level of sobriety.

43. A computing system, comprising:
one or more processors;
memory storing one or more programs to be executed by the one or more processor, the one or more programs comprising instructions for determining an eye closure status of a respective subject by a method comprising:
(a) obtaining, in electronic format, a first lower eyelid trace, wherein the first lower eyelid trace comprises, for each respective time increment in a plurality of consecutive time increments, a respective location of a lower eyelid of a first eye of the respective subject;
(b) obtaining, in electronic format, a first upper eyelid trace, wherein the first upper eyelid trace comprises, for each respective time increment in the plurality of consecutive time increments, a respective location of an upper eyelid of the first eye;
(c) obtaining, between a first and second time increment within the plurality of consecutive time increments, a first minimum difference between the location of the upper eyelid and the lower eyelid of the first eye across the time increments in the plurality of consecutive time increments that are between the first and second time increment;
(d) obtaining, between the first and second time increment within the plurality of consecutive time increments, a first maximum difference between the location of the upper eyelid and the lower eyelid of the first eye across the time increments in the plurality of consecutive time increments that are between the first and second time increment;
(e) passing (i) the first minimum difference and a (ii) difference between the first maximum difference and the first minimum difference through an activation function thereby obtaining a first result; and
(f) using the first result to provide an eye closure status of the first eye.

44. A non-transitory computer readable storage medium storing one or more programs for training a neural network to determine an eye closure status of a respective subject, the one or more programs configured for execution by a computer, wherein the one or more programs comprise instructions for:
(a) obtaining, in electronic format, a first lower eyelid trace, wherein the first lower eyelid trace comprises, for each respective time increment in a plurality of consecutive time increments, a respective location of a lower eyelid of a first eye of the respective subject;
(b) obtaining, in electronic format, a first upper eyelid trace, wherein the first upper eyelid trace comprises, for each respective time increment in the plurality of consecutive time increments, a respective location of an upper eyelid of the first eye;
(c) obtaining, between a first and second time increment within the plurality of consecutive time increments, a first minimum difference between the location of the upper eyelid and the lower eyelid of the first eye across the time increments in the plurality of consecutive time increments that are between the first and second time increment;
(d) obtaining, between the first and second time increment within the plurality of consecutive time increments, a first maximum difference between the location of the upper eyelid and the lower eyelid of the first eye across the time increments in the plurality of consecutive time increments that are between the first and second time increment;
(e) passing (i) the first minimum difference and a difference between the first maximum difference and (ii) the first minimum difference through an activation function thereby obtaining a first result; and
(f) using the first result to provide an eye closure status of the first eye.

45. The method of any one of claims 19-22, wherein the neural network comprises:
an initial convolutional neural network layer that receives one or more color pixel values for each pixel in the corresponding plurality of pixels as input into the neural network, wherein the initial convolutional neural network layer includes a first activation function, and wherein the initial convolutional neural network layer convolves the corresponding plurality of pixels into more than 10 separate parameters for each pixel in the corresponding plurality of pixels.

46. The method of claim 45, wherein the neural network further comprises a pooling layer that pools the 10 separate parameters for each pixel in the plurality of pixels outputted by the initial convolutional neural network layer.

47. The method of any one of claims 19-37, wherein the neural network includes a first portion and a second portion, and wherein the first portion of the neural network comprises an attention mechanism.

49. The method of claim 47, wherein the first portion of the neural network comprises an attention mechanism that further includes an encoder architecture.

50. The method of claim 47 or 48, wherein the attention mechanism is selected from the group consisting of global attention, self-attention, dot product attention, query-key-value attention, Luong attention, and Bahdanau attention.

51. The method of any one of claims 47-50, wherein the neural network is a transformer model.

52. The method of any one of claims 47-51, wherein the second portion of the neural network comprises a convolutional or graph-based neural network.

53. The method of any one of claims 1-42 or 45-52, wherein the activation function evaluates the first minimum difference and the difference between the first maximum difference and the first minimum difference as an Nth power of (i) the first minimum difference divided by (ii) the difference between the first maximum difference and the first minimum difference.

54. The method of claim 53, wherein N is a positive integer of 2 or greater.

55. The method of claim 53, wherein N is 2.

56. The method of any one of claims 1-42 or 45-55, wherein the activation function is a Sigmoid function.

57. The method of any one of claims 1-42 or 45-55, wherein the activation function is a logistic function, or a logit function.

58. The method of any one of claims 1-42 or 45-55, wherein the activation function is an ReLU function, a Softmax function, or a tanh function.
